(19) United States
(12) Patent Application Publication
(10) Pub. No.: US 2018/0110771 A1
(43) Pub. Date: Apr. 26, 2018

(54) Liposomal Irinotecan Preparations

(71) Applicant: Ipsen Biopharm Ltd., Wrexham (GB)

(72) Inventors: Daryl C. Drummond, Lincoln, MA (US); Dmitri B. Kirpotin, Revere, MA (US); Mark Eamon Hayes, Mill Valley, CA (US); Charles Noble, San Francisco, CA (US); Kevin Kesper, Watertown, MA (US); Antoine M. Awad, West Roxbury, MA (US); Douglas J. Moore, Newton, MA (US); Andrew J. O'Brien, Franklin, MA (US)

(21) Appl. No.: 15/645,645

(22) Filed: Jul. 10, 2017

Related U.S. Application Data
(63) Continuation of application No. 15/331,648, filed on Oct. 21, 2016, now abandoned.

(51) Int. Cl. A61K 31/4745; A61K 9/127; A61K 47/02

(52) U.S. Cl. CPC A61K 31/4745; Y10S 977/773; Y10S 977/907; Y10S 977/906; A61K 47/02; A61K 9/1277; A61K 9/127

(57) ABSTRACT

Irinotecan phospholipid liposomes with improved storage stability are provided, with related methods of treatment and manufacture. The irinotecan liposomes can have reduced formation of lyso-phosphatidylcholine (lyso-PC) during storage, and prior to administration to a patient.

### TECHNICAL FIELD

This disclosure relates to preparing pharmaceutical preparations containing liposomal irinotecan.

### BACKGROUND

Injectable liposome pharmaceutical products for the treatment of certain forms of cancer can be prepared by reacting irinotecan with liposomes containing a trapping agent, under conditions selected to retain the irinotecan in the liposomes with the trapping agent. For example, this process can be used to prepare dispersions of irinotecan liposomes comprising irinotecan and a trapping agent where the liposomes are formed from cholesterol and one or more phospholipid(s) (“PL”), such as phosphatidylcholine (“PC”). However, the storage of pharmaceutical preparations containing the liposomes comprising phospholipids and irinotecan can be limited by hydrolysis of the phospholipids. For example, the hydrolysis of PLs in the liposomes (such as PC) can lead to the formation of lyso-phosphatidylcholine (“lyso-PC”), which is a glycerylphosphocholine fatty acid monoester. Ultimately, excessive hydrolytic decomposition of a PL in the irinotecan liposomes can alter the release of the irinotecan compound from the liposomes during product storage, thereby limiting the storage life of the product.

The hydrolytic decomposition of liposomal irinotecan compositions comprising phospholipid(s) is affected by the pH of the composition. A pH of 6.0 or 6.5 is believed to minimize hydrolysis of phosphatidylcholine. However, the stability of phospholipid-containing liposomal irinotecan preparations at a pH of 6.5 was unexpectedly found to be adversely affected by the excessive formation of lyso-PC during storage under refrigerated conditions (2-8° C.). For example, irinotecan liposomes comprising irinotecan sucrose octasulfate encapsulated in liposomes formed from DSPC, cholesterol, and MPEG-2000-DSPE in a 3:2:0.015 mole ratio, at pH 6.5 (Sample 12), subsequently generated excessive levels of lyso-PC during refrigerated storage (2-8° C.) after manufacture.

Therefore, there remains a need for liposomal irinotecan preparations that do not form excessive amounts of lyso-PC during storage after manufacture. For example, there is a need for liposomal irinotecan preparations stabilized to reduce the amount of lyso-PC and/or rate of lyso-PC generation (with respect to total PL) during refrigerated storage of liposomal irinotecan at 2-8° C. after manufacturing.

### SUMMARY

The present invention provides novel liposomal irinotecan pharmaceutical compositions comprising ester-containing phospholipids with reduced rates of formation of lyso-phospholipid (“lyso-PL”) (e.g., lyso-phosphatidylcholine, or “lyso-PC”) during storage, after manufacturing. The present invention is based in part on the finding that liposomal irinotecan compositions can be manufactured that generate reduced amounts of lyso-phospholipids during storage for months at 2-8° C. In particular, the manufacture of such stabilized liposomal compositions is made possible in part by the unexpected finding that controlling specific parameters during liposome manufacture (e.g., the ratio of drug-to-phospholipid relative to the amount of trapping agent, the pH of the liposomal preparation and the amount of trapping agent cation in the liposomal preparation) synergistically reduces the formation of lyso-phospholipids during storage of liposomal irinotecan preparations. The invention provides extremely valuable information for designing and identifying improved irinotecan liposome compositions, which are more robust while reducing costs associated with the development of such compositions.

Some embodiments of the invention provide irinotecan liposome preparations comprising stabilized irinotecan liposomes encapsulating irinotecan sucrose octasulfate (SOS) in an unilamellar lipid bilayer vesicle formed from 1,2-distearoyl-sn-glycero-3-phosphocholine (DSPC), cholesterol, and a compound comprising polyethylene glycol and distearoylphosphatidyl ethanolamine (e.g., in a 3:2:0.015 mole ratio), obtained by a process comprising the steps of: (a) contacting a solution containing irinotecan with a trapping agent liposome encapsulating a triethylammonium (TEA) cation and sucrose octasulfate (SOS) as a trapping agent (e.g., TEA₈SOS at a sulfate concentration of 0.4-0.5 M) under conditions effective to load 500 g (±10% by weight) of the irinotecan moiety per mol phospholipid into the trapping agent liposome and removing all or substantially all of the TEA cation from the trapping agent liposomes, to form the irinotecan SOS liposomes, and (b) adjusting the pH of the composition comprising the irinotecan SOS liposomes to obtain a liposomal irinotecan preparation having a pH of 7.25-7.50. The process can include a pH adjustment by combining the irinotecan SOS liposomes with a suitable buffer system, such as 2-[4-(2-hydroxyethyl) piperazin-1-yl]ethanesulfonic acid (HEPES) (e.g., 10 mM HEPES). The stabilized irinotecan liposomes can have a diameter of about 110 nm (±20%), with a PDI less than 0.1. The compound comprising polyethylene glycol and distearoylphosphatidyl ethanolamine can be or otherwise comprise (for example) an alkyl-terminated polyethylene glycol-distearoylphosphatidyl ethanolamine of a suitable molecular weight (e.g., a methoxy-terminated polyethylene glycol (MW 2000)-distearoylphosphatidyl ethanolamine, or “MPEG-2000-DSPE”), a polyethylene glycol (MW 2000)-distearoylphosphatidyl ethanolamine (“PEG-2000-DSPE”), a suitable copolymer of polyethylene glycol and distearoylphosphatidyl ethanolamine, and the like.

The irinotecan liposomes in the resulting liposomal irinotecan preparation are stabilized to have less lyso-phosphatidylcholine (lyso-PC) (e.g., less than 10 mol % lyso-PC with respect to the total amount of phosphatidylcholine in the irinotecan liposomes during the first 3 months of storage at about 4° C. after manufacturing). Preferably, the stabilized liposomal irinotecan comprising one or more phospholipid(s) (including PEG-containing phospholipid(s)), form not more than 10 mol % of lyso-PL (relative to the total phospholipids) during storage for the first month of storage at about 4° C., and/or form not more than 20 mol % of lyso-PL (relative to the total phospholipids) during storage for the first 6 months of storage at about 4° C. and/or not more than 25 mol % of lyso-PL during storage for the first 9 months of storage at about 4° C. These stabilized irinotecan liposomes preferably form lyso-PC at an average rate of less than about 2 mol % (e.g., 0.5-1.5 mol %) lyso-PC per month during the first 9 months of storage at about 4° C., following manufacture of the liposomal irinotecan compositions. Stabilized phosphatidylcholine-containing liposomal irinotecan compositions can generate less than 1 mg lyso-PC per mL of the liposomal irinotecan preparation during the first 9 months of stability testing at 2-8° C. after manufacturing, more preferably less than about 0.1 mg/mL lyso-PC formation per month during the first 9 months of stability testing at 2-8° C. after manufacturing. In a preferred embodiment, the stabilized phosphatidylcholine-containing liposomal irinotecan compositions generate less than 1 mg lyso-PC during the first 9 months of stability testing at about 4° C. after manufacturing, more preferably less than about 0.1 mg/mL lyso-PC per month during the first 9 months of stability testing at about 4° C. after manufacturing.

The irinotecan liposomes in the resulting liposomal irinotecan preparation can also be stabilized to form less lyso-phosphatidylcholine (lyso-PC) (e.g., forming less than 10 mol % lyso-PC with respect to the total amount of phosphatidylcholine in the irinotecan liposomes during the first 3 months of storage at about 4° C. after manufacturing). Preferably, the liposomal irinotecan preparations have less than 0.5 mg/mL lyso-PC when manufactured, and form lyso-PC at a rate resulting in a measurement of less than 2 mg/mL lyso-PC in the liposomal irinotecan preparation after storage for at least 12-36 months at 2-8° C. In a preferred embodiment, the stabilized phosphatidylcholine-containing liposomal irinotecan compositions generate less than 1 mg lyso-PC during the first 9 months of stability testing at about 4° C. after manufacturing, more preferably less than about 0.1 mg/mL lyso-PC per month during the first 9 months of stability testing at about 4° C. after manufacturing.

In a first embodiment, stabilized liposomal irinotecan compositions are obtained by a process characterized by using certain preferred ratios of the liposomal irinotecan, an anionic trapping agent, and liposome-forming phospholipids during the manufacturing of the irinotecan liposomes, according to a Stability Ratio (“SR”) that is greater than about 950 (e.g., prepared with a SR of 990-1500). The Stability Ratio, is defined as follows: SR=A/B, where: A is the amount of irinotecan moiety encapsulated in trapping agent liposomes during the irinotecan drug loading process, in grams equivalent to the irinotecan free anhydrous base, per mole of phospholipid in the composition; and B is the concentration of sulfate groups in the trapping agent solution (e.g., sucrosofate or other trapping agent) used to make the trapping agent liposomes, expressed in mole/L (based on the concentration of sulfate groups). This embodiment of the invention is based in part on the discovery that when phospholipid-based liposomal irinotecan preparations are made by reacting (1) irinotecan with (2) liposomes encapsulating an anionic trapping-agent (e.g., sucrose octasulfate), the stability of the resulting irinotecan-loaded liposomes depends on the ratio of the component reactants, as expressed by the applicable Stability Ratio.

Preparing phospholipid-containing irinotecan liposomes using reactants providing (i) a concentration of trapping agent solution used to make a trapping agent liposome, (ii) an amount of phospholipids in the liposomes and (iii) an amount of irinotecan together selected to provide Stability Ratios of at least about 990 (e.g., 990-1500, preferably about 1000-1200) dramatically reduces the amount of lyso-PL formed in the resulting liposomal irinotecan preparations during refrigerated storage, thereby extending the storage time for these compositions. Phospholipid-containing liposomal irinotecan preparations manufactured with a Stability Ratio greater than about 950 produced surprisingly less lyso-PC, even at pH 6.5, compared to liposomal irinotecan preparations manufactured with lower Stability Ratios. For example, phosphatidylcholine-containing irinotecan liposomes prepared at pH 6.5 with a Stability Ratio of about 942 (Sample 3) generated about 36 mol % lyso-PC, compared to about 24 mol % lyso-PC generated in irinotecan liposomes prepared with a Stability Ratio of about 990 (Sample 2), during 9 months of storage at 4° C. (i.e., increasing the Stability Ratio by about 5% resulted in a 34% reduction in lyso-PC generation in these two liposomal irinotecan preparations prepared at the same pH). In contrast, increasing the Stability Ratio from 724 (Sample 12) to 942 (Sample 3) below the threshold of 950 at pH 6.5 did not significantly change the amount of lyso-PC generated in these samples during the first 9 months of storage at 4° C. See Table 1 (compare 35.7 mol % lyso-PC for Sample 3 to 35.4 mol % lyso-PC for Sample 12).

In a second embodiment, stabilized liposomal irinotecan compositions are obtained by a process that includes providing a pH greater than 6.5, resulting in a reduction in subsequent formation of lyso-PC in the liposomal irinotecan during storage stability testing. Increasing the pH of liposomal irinotecan preparations above 6.5 (e.g., 6.8-7.6, preferably 7.0-7.5) also reduces the amount of lyso-PL formed in the resulting liposomal irinotecan preparations during refrigerated storage, thereby extending the storage time for these compositions. Preferably, a stabilized liposomal irinotecan composition has a pH greater than 6.5 (e.g., 7.0-7.5, including 7.25, 7.3 and 7.5) after manufacturing and prior to product storage. Preferably, the liposomal irinotecan preparations are obtained by a process with a SR of at least about 950 (e.g., 990-1200) and a final pH of 7-7.5 (e.g., 7.0, 7.25, 7.3 or 7.5) after manufacturing and prior to storage.

The liposomal irinotecan compositions are useful in the treatment of patients diagnosed with various forms of cancer. For example, liposomal irinotecan can be administered for the treatment of small cell lung cancer (SCLC) without other antineoplastic agents. In some embodiments, the liposomal irinotecan compositions are administered in combination with other antineoplastic agents. For example, a liposomal irinotecan composition, 5-fluorouracil, and leucovorin (without other antineoplastic agents) can be administered for treatment of patients diagnosed with metastatic adenocarcinoma of the pancreas with disease progression following gemcitabine-based therapy. A liposomal irinotecan composition, 5-fluorouracil, leucovorin, and oxaliplatin (without other antineoplastic agents) can be administered for treatment of patients diagnosed with previously untreated pancreatic cancer. A liposomal irinotecan composition, 5-fluorouracil, leucovorin, and an EGFR inhibitor (e.g., an oligoclonal antibody EGFR inhibitor such as MM-151) can be administered for treatment of patients diagnosed with colorectal cancer.

Unless otherwise indicated, all of the irinotecan liposomes in Example 1 and Example 2 (as well as data in Table 1, Table 2, Table 4A, and Table 4B) were prepared using an amount of irinotecan moiety (as explained above, based on the free base anhydrous) to total phospholipid ratio of about 471 g irinotecan moiety (equivalent to the amount of irinotecan moiety in about 500 g anhydrous irinotecan HCl salt) per mole total phospholipid, respectively. Unless otherwise indicated, the measurement of lyso-PC in the irinotecan liposomes in Example 1, Example 2 and Example 7 (as well as data in Table 1, Table 2, Table 4A, Table 4B, and Table 8) were obtained using the TLC Method B described herein.

Unless otherwise indicated, the irinotecan liposomes in Example 3, Example 5, and Example 6 (as well as data in Table 5, Table 6, and Table 7) were prepared using an irinotecan moiety (as explained above, based on the free base anhydrous) to total phospholipid ratio of 500 g (±15%) irinotecan moiety (using an equivalent amount of irinotecan HCl salt in a solution) per mole total phospholipid, respectively. Unless otherwise indicated, the measurement of lyso-PC in the irinotecan liposomes in Example 3, Example 5, and Example 6 (as well as data in Table 5, Table 6, and Table 7) were obtained using the HPLC Method A described in Example 8.

As used herein (and unless otherwise specified), “DLS” refers to dynamic light scattering and “BDP” refers to bulk drug product.

In some embodiments, the liposomes of the present invention encapsulate one or more agents that trap the pharmaceutical drug within liposomes (hereafter referred to as trapping agents).

Unless otherwise indicated, liposomal preparations can comprise (e.g., spherical or substantially spherical) vesicles with at least one lipid bilayer, and may optionally include a multilamellar and/or unilamellar vesicles, and vesicles that encapsulate and/or do not encapsulate pharmaceutically active compounds (e.g., irinotecan) and/or trapping agent(s). For example, unless otherwise indicated, a pharmaceutical liposomal preparation comprising irinotecan liposomes may optionally include liposomes that do not comprise irinotecan, including a mixture of unilamellar and multilamellar liposomes with or without irinotecan and/or trapping agent(s).

### BRIEF DESCRIPTION OF THE FIGURES

FIG. 1 is a bar graph of the mol % lyso-PC measured in liposomal irinotecan preparations obtained using processes of Example 1 having different Stability Ratios. Liposomal irinotecan preparations were stored up to 9 months at 4° C. The mol % lyso-PC (with respect to total phospholipid) was measured by TLC Method B, below.

FIG. 2A is a bar graph of the mol % lyso-PC measured in liposomal irinotecan preparations obtained using the process of Example 1 having the same Stability Ratio (785) but different pH levels. The liposomal irinotecan preparations were stored up to 9 months at 4° C. The pH of the samples after manufacture, but before storage, were as follows: Sample 9 (pH 6.5), Sample 10 (pH 7.25) and Sample 11 (pH 7.5). The mol % lyso-PC (with respect to total phospholipid) lyso-PC was measured by TLC Method B, below.

FIG. 2B is a bar graph of the mol % lyso-PC in liposomal irinotecan preparations obtained using the process of Example 1 at the same Stability Ratio (1047) but different pH levels. Liposomal irinotecan preparations were stored up to 9 months at 4° C. The pH of the samples after manufacture, but before storage, were as follows: Sample 1 (pH 6.5), Sample 5 (pH 7.25), Sample 6 (pH 6.5), Sample 7 (pH 7.25), Sample 8 (pH 7.5), and Sample 13 (pH 7.25). The mol % lyso-PC (with respect to total phospholipid) was measured by TLC Method B, below.

FIG. 3A is a scatter plot graph of the Stability Ratio values versus the mol % lyso-PC in irinotecan liposome compositions of Table 1 (Samples 1-12) and Table 4A (Sample 13-14) after 9 months of storage at 4° C. The sample pH values after manufacture, but before storage, are indicated. The mol % lyso-PC (with respect to total phospholipid) was measured by TLC Method B, below.

FIG. 3B is a graph of the Stability Ratio values versus the mol % lyso-PC in irinotecan liposome compositions of Table 1 (Samples 1-12) after 6 months of storage at 4° C. The sample pH values after manufacture, but before storage, are indicated. The mol % lyso-PC (with respect to total phospholipid) was measured by TLC Method B, below.

FIG. 4A is a graph of the concentration of lyso-PC (mg/mL) versus the months of storage at 4° C. of three irinotecan liposome compositions described in Example 3. The mol % lyso-PC (with respect to total phospholipid) was measured by HPLC Method A (Example 8).

FIG. 4B is a graph of the concentration of lyso-PC (mg/mL) versus the months of storage at 4° C. in three irinotecan liposome described in Example 3. The mol % lyso-PC (with respect to total phospholipid) was measured by HPLC Method A (Example 8).

FIG. 5 is a graph of the estimated rate of lyso-PC (mg/mL/month) formation during storage at 4° C. in irinotecan liposome compositions described in Example 3 having various amounts of substituted ammonium (protonated TEA). The mol % lyso-PC (with respect to total phospholipid) was measured by HPLC Method A (Example 8).

FIG. 6 shows a graph plotting the average particle size of 12 different irinotecan sucrose octasulfate liposome compositions described in Example 6 for various product lot numbers (i.e., vial number) stored over a period of up to 36 months at 4° C., with linear regressions to the data obtained for each sample.

FIG. 7 is a graph of the particle size polydispersity index (PDI) of the irinotecan sucrose octasulfate liposome compositions described in Example 6 for various product lot numbers (i.e., vial number) shown in FIG. 6, with linear regressions to the data obtained for each sample.

FIG. 8 is a graph of the pH of 13 different irinotecan sucrose octasulfate liposome compositions described in Example 6 for various product lot numbers (i.e., vial number) stored over a period of up to 36 months at 4° C., with linear regressions to the data obtained for each sample.

FIG. 9A is a graph of the mol % lyso-PC for 16 different irinotecan sucrose octasulfate liposome compositions described in Example 6 for various product lot numbers stored over a period of 12 months or more at 4° C., with linear regressions to the data obtained for each sample. The mol % lyso-PC (with respect to total phospholipid) was measured by HPLC Method A (Example 8).

FIG. 9B is a graph of the concentration of lyso-PC (mg/mL) over 36 months in two irinotecan sucrose octasulfate liposome compositions, and best-fit linear regressions to the respective data points obtained from each irinotecan liposome sample. The mol % lyso-PC (with respect to total phospholipid) was measured by HPLC Method A (Example 8).

FIG. 9C is a bar graph showing the concentration of lyso-PC (mg/mL) measured in three liposomal irinotecan sucrose octasulfate preparations described in Example 6. The mol % lyso-PC (with respect to total phospholipid) was measured by HPLC Method A (Example 8).

FIG. 10 is a graph of the gram-equivalent amounts of irinotecan and sucrosofate in the precipitate formed by combining, in aqueous solution, irinotecan hydrochloride trihydrate and triethylammonium sucrosofate in various proportions, i.e., in gram-equivalent ratios from 1:9 to 9:1. The x-axis shows the relative gram-equivalent overall amount of triethylammonium sucrosofate (SOS) in the samples, in reference to the gram-equivalent of irinotecan free base anhydrous.

FIG. 11A is a representative chromatogram for Method A at full scale.

FIG. 11B is a representative chromatogram for Method A at enlarged scale.

### DETAILED DESCRIPTION

The liposome manufacturing processes disclosed herein can provide stabilized irinotecan liposome compositions comprising ester-containing phospholipids with reduced formation of lyso-PC during storage at 2-8° C., such as at about 4° C. In general, the stabilized irinotecan liposomes can be obtained from a process comprising (a) forming a trapping agent liposome encapsulating an anionic trapping agent and a cation within a liposome vesicle comprising phospholipid(s) (thereby forming trapping agent liposomes), and (b) subsequently contacting the trapping agent liposomes with a solution comprising an irinotecan moiety (e.g., irinotecan free base, a dissolved hydrochloride salt of irinotecan, or the like) under conditions effective to load the irinotecan moiety into the trapping agent liposome and retain the irinotecan inside the liposome with the trapping agent to form the irinotecan liposomes. The irinotecan and the trapping agent can form a precipitate (e.g., a salt or amorphous composition) within the liposome during and/or after the drug loading process. Preferably, the cation is removed or substantially removed from the trapping agent liposomes during the irinotecan drug loading process. For example, stabilized irinotecan liposomes can be prepared by a process that includes the steps of (a) preparing a trapping agent liposome containing triethylamine (TEA) cation as a triethylammonium form of sucrosofate (e.g., TEA₈SOS), and (b) subsequently contacting the trapping agent liposomes with irinotecan under conditions effective for a desired amount of the irinotecan to enter the liposome and to permit a corresponding amount of the trapping agent cation (e.g., TEA) to leave the liposome (thereby exhausting or reducing the concentration gradient of TEA across the resulting liposome). Irinotecan sucrose octasulfate can form within the liposomes during the drug loading process. Preferably, the liposomal irinotecan compositions contain a minimal amount of residual material derived from the trapping agent cation (e.g., less than about 100 ppm TEA) after manufacturing (e.g., after completion of the drug loading process). The pH of a liposomal irinotecan preparation containing the irinotecan liposomes is preferably selected (e.g., above 6.5, such as 6.8-7.6 and preferably 7.0-7.5) to reduce the formation of lyso-PC during refrigerated storage (e.g., about 4° C.). The liposomal irinotecan preparations can be obtained by a process described herein (e.g., using processes disclosed in Example 1, Example 6, or modifications thereof).

The present invention is based in part on a number of unexpected observations. First, irinotecan-SOS liposome compositions surprisingly formed substantially less lyso-PC during refrigerated storage when the amount of encapsulated irinotecan was increased relative to the amount of co-encapsulated SOS trapping agent. Second, irinotecan-SOS liposome compositions surprisingly have less lyso-PC during refrigerated storage when the pH of the aqueous medium containing the irinotecan-SOS liposomes after manufacture but prior to storage is above 6.5. Third, irinotecan-SOS liposome compositions surprisingly have less lyso-PC when the amount of residual liposomal trapping agent ammonium/substituted ammonium cation assayed in the composition is below 100 ppm.

Applicants have discovered that when phospholipid-based irinotecan-containing liposomes are made by reacting irinotecan with trapping agent liposomes encapsulating a trapping-agent to load or otherwise capture and retain irinotecan within the trapping agent liposomes, the stability of the resulting irinotecan liposomes depends on the ratio of (A) the initial concentration of the trapping agent used to make the trapping agent liposomes to (B) the weight amount of the irinotecan (grams) per total moles of phospholipids loaded into the irinotecan liposomes. This ratio is defined herein as the Stability Ratio. Surprisingly, phosphatidylcholine-containing irinotecan liposomes having a Stability Ratio of greater than about 950 (e.g., 950-1500, preferably about 1000-1200) exhibited reduced lyso-PC formation during refrigerated storage at 2-8° C.

As used herein, the Stability Ratio (“SR”) is defined as follows:

SR=A/B,

where:
a. A is the amount of irinotecan moiety encapsulated in trapping agent liposomes during the drug loading process, in grams equivalent to the irinotecan free anhydrous base, per mole of phospholipid in the composition; and
b. B is the concentration of sulfate groups in the sucrosofate (or other trapping agent) solution used to make the trapping agent liposomes, expressed in mole/L (based on the concentration of sulfate groups).

With respect to the determination of the Stability Ratio, the number of moles of phospholipid in the liposome preparation is determined by assay, such as described in the Examples. The irinotecan moiety amount (A above) is calculated accordingly for conducting liposome loading. Also with respect to the determination of the Stability Ratio, the concentration B of sulfate groups in the sucrosofate (or other trapping agent) solution, expressed in mole/L, is calculated as the concentration of sulfate groups in sucrosofate (or other trapping agent disclosed herein) (in mole/L) in the solution that is added to lipids (which are typically dissolved in alcohol, typically in a volume that is 10% or less than the volume of the trapping agent solution added to the lipids). Thus for sucrosofate, the concentration B of sulfate groups is the concentration of sucrosofate multiplied by 8 (i.e., the number of sulfate groups in one sucrosofate molecule), or multiplied in accordance with the number of sulfate groups of the particular trapping agent used. (See Example 1.)

Storage stabilized irinotecan liposomes can be prepared in multiple steps comprising the formation of a trapping agent liposome, followed by loading of irinotecan into the trapping agent liposome (e.g., as the trapping agent cation leaves the trapping agent liposome). In general, the trapping agent liposome can be obtained by hydrating and dispersing the liposome lipids in a solution of the trapping agent to form trapping agent liposomes. The trapping agent liposomes can be prepared from a solution of an ionic salt of the trapping agent comprising an anion (or polyanion) and a cation counter-ion. In some examples, the trapping agent liposomes are prepared in the absence of irinotecan, and do not contain irinotecan prior to the drug loading step. For example, trapping agent liposomes can be obtained by dissolving the lipids, including DSPC and cholesterol, in heated ethanol, and dispersing the dissolved and heated lipid solution in an aqueous solution of the trapping agent cation and anion (e.g., TEA-sucrosofate) at a temperature above the transition temperature (Tₘ) of the liposome lipid, e.g., 60° C. or greater. The lipid dispersion can be formed into liposomes having the average size of 75-125 nm (such as 80-120 nm, or in some embodiments, 90-115 nm), by extrusion through track-etched polycarbonate membranes with the defined pose size, e.g., 100 nm. The irinotecan liposomes are preferably about 100±20%, such as 100 nm±20 nm, preferably 100±10 nm, in diameter.

The trapping agent anion can be a polyanionic compound with a plurality of negatively charged groups or comprise a combination of two or more different such compounds. In some embodiments, the trapping agent is a sulfated sugar and/or polyol. Non-limiting examples of sulfated sugar trapping agents are sulfated sucrose compounds including, without limitation, sucrose hexasulfate, sucrose heptasulfate, and sucrose octasulfate (SOS). Exemplary polyol trapping agents include inositol polyphosphates, such as inositol hexaphosphate (also known as phytic acid or IHP) or sulfated forms of other disaccharides. In a preferred embodiment of the present invention, the trapping agent is sucrose octasulfate (SOS). Sucrosofate is also referred to as sucrose octasulfate or sucro-octasulfate. Sucrose octasulfate (also referred to as sucrosofate), is a fully substituted sulfate ester of sucrose having, in its fully protonated form, the structure of formula (I):

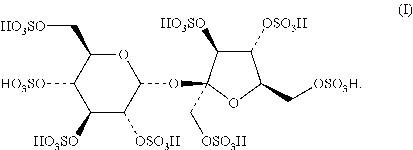

Methods of preparing sucrosofate in the form of various salts, e.g., ammonium, sodium, or potassium salts, are well known in the field (see, e.g., U.S. Pat. No. 4,990,610, which is incorporated by reference herein in its entirety). Likewise sulfated forms of other disaccharides, for example, lactose and maltose, to produce lactose octasulfate and maltose octasulfate, are envisioned.

The cation of the trapping agent liposomes can be encapsulated or otherwise retained with the trapping agent anion of the trapping agent liposomes, in an amount effective to provide for the desired loading of irinotecan into the trapping agent liposomes when heated above the phase transition temperature of the lipid components of the trapping agent liposome. The trapping agent liposome cations are selected to leave the trapping agent liposomes when reacted with irinotecan under conditions effective to load the irinotecan into the trapping agent liposomes. The trapping agent cation can be ammonium or a substituted ammonium compound. Preferably, the substituted ammonium trapping agent cation compound has a pKa of at least about 8.0. In some embodiments of the invention, the substituted ammonium compound has a pKa of at least about 8.0, at least about 8.5, at least about 9.0, at least 9.5, at least 10.0, at least 10.5, or at least 11.0 as determined in an aqueous solution at ambient temperature. In some embodiments of the invention, the substituted ammonium compound has a pKa of about 8.0-12.0, about 8.5.-11.5, or about 9.0-11. In a preferred embodiment, the trapping agent liposome comprises triethylammonium (TEA) and/or diethylammonium (DEA) cations. The cations can be selected so that they can leave the trapping agent liposomes during the loading of the irinotecan into the liposomes.

For example, trapping agent liposomes can be formed using a solution of TEA as the trapping agent cation and SOS anionic trapping agent comprising at least 8 molar equivalents of TEA to each molar equivalent of sucrosofate (SOS) in a solution that can have a sulfate concentration of about 0.40-0.50 M, and a pH (e.g., about 6.5), selected to prevent unacceptable degradation of the liposome phospholipid during the dispersion and extrusion steps (e.g., a pH selected to minimize the degradation of the liposome phospholipid during these steps).

The trapping agent liposomes (converted to subsequently-formed irinotecan liposomes after drug loading) can be made from a variety of lipids, especially phospholipids, known in the art that can be constituents of the trapping agent liposomes and the irinotecan liposomes, such as phosphatidyl ethanolamine, and phosphatidyl serine, and it is within the skill in the art to make liposomes with other such phospholipids. In some embodiments, liposomes of the present inventions are composed of 1,2-distearoyl-sn-glycero-3-phosphocholine (DSPC), cholesterol, and a PEG-containing co-polymer of DSPC, such as methoxy-terminated polyethylene glycol (MW 2000)-distearoylphosphatidyl ethanolamine (MPEG-2000-DSPE). Below are described preferred embodiments regarding the lipids present in liposome preparations disclosed herein.

The liposomal components can be selected to produce the liposomal bilayer membrane which forms unilamellar and/or multilamellar vesicles encapsulating and retaining the active substance until it is delivered to the tumor site. Preferably, the liposome vesicles are unilamellar. The liposomal components are selected for their properties when combined to produce liposomes capable of actively loading and retaining the active substance while maintaining low protein binding in vivo and consequently prolonging their circulation lifetime.

DSPC is preferably the major lipid component in the bilayer of the liposome encapsulating irinotecan (e.g., comprising 74.4% of total weight of all lipid ingredients). DSPC has a phase transition temperature (Tm) of 55° C.

Cholesterol can preferably comprise about 24.3% of total weight of all lipid ingredients. It can be incorporated in an amount effective to stabilize liposomal phospholipid membranes so that they are not disrupted by plasma proteins, to decrease the extent of binding of plasma opsonins responsible for rapid clearance of liposomes from the circulation, and to decrease permeability of solutes/drugs in combination with bilayer forming phospholipids.

MPEG-2000-DSPE or other suitable PEG-ylated phospholipids (e.g., PEG or MPEG co-polymers with DSPE having the same or other appropriate molecular weights) can preferably comprise about 1.3% of total weight of all lipid bilayer constituents. Its amount and presence on the surface of the irinotecan liposome can be selected to provide a minimal steric barrier preventing liposome aggregation. The MPEG-2000-DSPE coated liposomes of the present invention are shown to be stable with respect to size and drug-encapsulation.

In some embodiments, the lipid membrane of the trapping agent liposomes and the irinotecan liposomes are preferably composed of the following ingredients: 1, 2-distearoyl-sn-glycero-3-phosphocholine (DSPC), cholesterol, and methoxy-terminated polyethylene glycol (MW 2000)-distearoylphosphatidyl ethanolamine (MPEG-2000-DSPE), in the ratio of approximately one polyethylene glycol (PEG)-modified phospholipid molecule for every 200 non-PEG-phospholipid molecules and/or total liposome DSPC and cholesterol combined at a 3:2 mol ratio. In preferred embodiments, trapping agent liposomes and/or irinotecan liposomes of the present invention are made from a mixture of DSPC, cholesterol, and MPEG-2000-DSPE combined in a 3:2:0.015 molar ratio. In preferred embodiments, liposome preparations of the present invention consist of 1,2-distearoyl-sn-glycero-3-phosphocholine (DSPC) (e.g., at a concentration of about 6.81 mg/mL), cholesterol (e.g., at a concentration of about 2.22 mg/mL), and methoxy-terminated polyethylene glycol (e.g., (MW 2000)-distearoylphosphatidyl ethanolamine (MPEG-2000-DSPE) at a concentration of about 0.12 mg/mL). In more preferred embodiments, irinotecan liposomes of the present invention include 1,2-distearoyl-sn-glycero-3-phosphocholine (DSPC) at a concentration of 6.81 mg/mL, cholesterol at a concentration of 2.22 mg/mL, and methoxy-terminated polyethylene glycol (MW 2000)-distearoylphosphatidyl ethanolamine (MPEG-2000-DSPE) at a concentration of 0.12 mg/mL. The trapping agent liposomes and/or the irinotecan liposomes can contain DSPC and cholesterol in a weight ratio of about 2-4 to 1, but preferably about 3 to 1. More preferably, the trapping agent liposomes comprise DSPC and cholesterol in about a 3:2 mole ratio.

Once the trapping agent liposomes are formed, trapping agent cations and anions that are not encapsulated or otherwise retained in the trapping agent liposomes can be removed prior to loading of irinotecan into the trapping agent liposomes. The trapping agent cation retained by the trapping agent liposomes can be subsequently removed and/or replaced by the irinotecan loaded into the liposome in a subsequent drug loading step, described below. The non-entrapped trapping agent (e.g., TEA₈SOS) can be removed from the liposome dispersion, e.g., by dialysis, gel chromatography, ion exchange, or ultrafiltration prior to irinotecan encapsulation.

Preferably, the trapping agent liposomes have a transmembrane concentration gradient of a membrane-traversing cation, such as ammonium or substituted ammonium, effective to result in the exchange of the ammonium/substituted ammonium in the trapping agent liposomes for the irinotecan when heated above the phase transition temperature of the lipid components of the liposomes. Preferably, the trapping agent has a higher concentration in the trapping agent liposome than in the media surrounding it. In addition, the trapping agent liposomes can include one or more trans-membrane gradients in addition to the gradient created by the ammonium/substituted ammonium cation. For example, the liposomes contained in the trapping agent liposome composition can additionally or alternatively include a transmembrane pH gradient, ion gradient, electrochemical potential gradient, and/or solubility gradient. Extra-liposomal cations can be removed after the preparation of the liposomes loaded with irinotecan.

The trapping agent liposomes can be converted into stabilized irinotecan liposomes in one or more drug loading step(s) by heating the trapping agent liposomes above the phase transition temperature of the phospholipids in the trapping agent liposomes, in the presence of a solution comprising irinotecan. Preferably, the irinotecan is loaded into the trapping agent liposomes by incubating the irinotecan with the trapping agent liposomes in an aqueous medium at a suitable temperature, e.g., a temperature above the primary phase transition temperature of the component phospholipids during loading, while being reduced below the primary phase transition temperature of the component phospholipids after loading the irinotecan, preferably at about room temperature. The incubation time is usually based on the nature of the component lipids, the irinotecan to be loaded into the liposomes, and the incubation temperature. Typically, the incubation times of several minutes (for example 30-60 minutes) to several hours are sufficient.

The concentration of the trapping agent and the amount of phospholipid material used to form the trapping agent liposomes are preferably selected in combination with the amount of irinotecan loaded into the trapping agent liposome and the amount of phospholipids in the trapping agent liposome, to provide a Stability Ratio of greater than about 950, as defined above.

The trapping agent liposomes can be contacted with a solution of irinotecan under conditions effective to load the irinotecan into the liposomes (e.g., heating the liposomes above the transition temperature of the phospholipids in the liposomes), as described in the Examples. The irinotecan or a salt thereof is added to trapping agent liposomes containing one or more trapping agents, where the irinotecan is present at a concentration of irinotecan moiety equivalent to, in grams of the irinotecan free anhydrous base, 200 g, 300 g, 400 g, 500 g, 600 g, or 700 g per mol phospholipid. In some embodiments, the irinotecan is present during the drug loading process at a concentration of irinotecan moiety equivalent to, in grams of the irinotecan free anhydrous base from 200 to 300 g, from 400 to 550 g, from 450 to 600 g, or from 600 to 700 g per mol phospholipid. Preferably, about 500 g (±15%) moiety loaded into irinotecan liposomes per mol phospholipid, including 471 g irinotecan moiety per mol total phospholipid. Specific examples herein include measurements of stabilized irinotecan liposomes containing 471 g irinotecan moiety per mol total phospholipid, as well as irinotecan liposomes containing 500 g irinotecan moiety per mol total phospholipid.

Preferably, the irinotecan liposomes are prepared from trapping agent liposomes (e.g., SOS and/or another sulfated polyol trapping agent, including acceptable salts thereof) obtained from a solution with the anionic trapping agent at a concentration of about 0.4-0.5 M sulfate groups, e.g. these specific values ±10%, when the irinotecan liposomes are loaded with about 500 g irinotecan per mol total liposome phospholipid, e.g. these specific values ±10%. The trapping agent used for the preparation of liposomes (e.g., SOS and/or another sulfated polyol trapping agent, including acceptable salts thereof) can have a concentration of 0.3-0.8, 0.4-0.05, 0.45-0.5, 0.45-0.0475, 0.45-0.5, 0.3, 0.4, 0.45, 0.475, 0.5, 0.6, 0.7, or 0.8 M sulfate groups, e.g. these specific values ±10%. In a preferred embodiment, the trapping agent used for the preparation of liposomes is SOS and has a concentration of from about 0.4 to about 0.5 M sulfate groups. In a preferred embodiment, the trapping agent used for the preparation of the trapping agent liposomes is SOS and has a concentration of about 0.45 M or about 0.475 M sulfate groups. The trapping agent liposomes are loaded with a total of 500 g (±10%) irinotecan per mol total phospholipid to obtain the irinotecan liposomes. In a preferred embodiment, the trapping agent used for the preparation of trapping agent liposomes is SOS and has a concentration of about 0.45 M sulfate groups (±10%) and a TEA or DEA trapping agent cation, to obtain an liposomes prepared according to Example 1 having a diameter of about 110 nm (measured by quasi-elastic light scattering) containing a total of 500 g (±10%) irinotecan per mol total phospholipid and less than about 100 ppm residual TEA or DEA.

These liposomes can be stabilized by loading enough irinotecan into the liposomes to reduce the amount of trapping agent cation (e.g., TEA) in the resulting irinotecan liposome composition to a level that results in less than a given maximum level of lyso-PC formation after a period of storage time (e.g., 6 months or 9 months) at about 4° C., or, at 5±3° C., measured, e.g., in mg/mL/month, or mol % PC conversion into a lyso-PC over a unit time, such as, mol % lyso-PC/month. Next, the trapping agent cation (e.g., TEA) can be exchanged from the liposomes into the external medium during the loading process, along with any unentrapped irinotecan, typically removed from the liposomes by any suitable known process(es) (e.g., by gel chromatography, dialysis, diafiltration, ion exchange or, ultrafiltration). The liposome external medium can be exchanged for an injectable isotonic fluid (e.g. isotonic solution of sodium chloride), buffered at a desired pH to obtain the liposomal irinotecan preparation comprising stabilized irinotecan liposomes.

The irinotecan loading of the trapping agent liposomes can be conducted in an aqueous solution at the ionic strength of less than that equivalent to 50 mM NaCl, or more preferably, less than that equivalent to 30 mM NaCl. After drug loading, a more concentrated salt solution, e.g., NaCl solution, may be added to raise the ionic strength to higher than that equivalent to 50 mM NaCl, or more preferably, higher than that equivalent to 100 mM NaCl, preferably equivalent to between about 140-160 mM NaCl.

Because high entrapment efficiencies of more than 85%, typically more than 90%, are achieved, there is often no need to remove unentrapped entity. If there is such a need, however, the unentrapped irinotecan can be removed from the composition by various means, such as, for example, size exclusion chromatography, dialysis, ultrafiltration, adsorption, and precipitation. Extra-liposomal irinotecan can be removed after the loading of the irinotecan into the liposomes. Preferably, at least 95% (preferably, 98% or 99%) of the irinotecan in the liposomal irinotecan preparation is encapsulated within the irinotecan liposomes. Preferably, the liposomal irinotecan preparation contains less than 2% unencapsulated irinotecan.

Reducing the amount of (e.g., residual) trapping agent cation (e.g., protonated substituted ammonium cation) in the irinotecan liposome after drug loading can reduce the formation of lyso-PC. In a preferred embodiment, the concentration of the trapping agent cation (e.g., ammonium or substituted ammonium cation) within the irinotecan liposomes is low enough to provide low amounts of lyso-PC after refrigerated storage for prolonged periods of the irinotecan liposome preparations. For example, reduction in the amount of lyso-PC formation was observed in irinotecan SOS liposome preparations having less than about 100 ppm of the substituted ammonium cation (herein ppm are reported for the substituted amine corresponding to the substituted ammonium), preferably between 20 and 80 ppm, preferably less than about 50 ppm, even more preferably less than about 40 ppm, still more preferably less than 30 ppm. In some embodiments, the irinotecan SOS liposomes (such as Samples 24-29) comprise less than 100 ppm, or about 15-100 ppm substituted ammonium SOS trapping agent counter ion. In some embodiments, the irinotecan SOS liposomes (such as Samples 24-29) comprise about 15-80 ppm substituted ammonium. In some embodiments, irinotecan SOS liposomes comprise about 40-80 ppm substituted ammonium. In some embodiments, the irinotecan SOS liposomes (such as Samples 24-29) comprise about 80-100 ppm substituted ammonium. In a preferred embodiment, the substituted ammonium present at any of the above-mentioned ppm concentrations is derived from TEA or DEA.

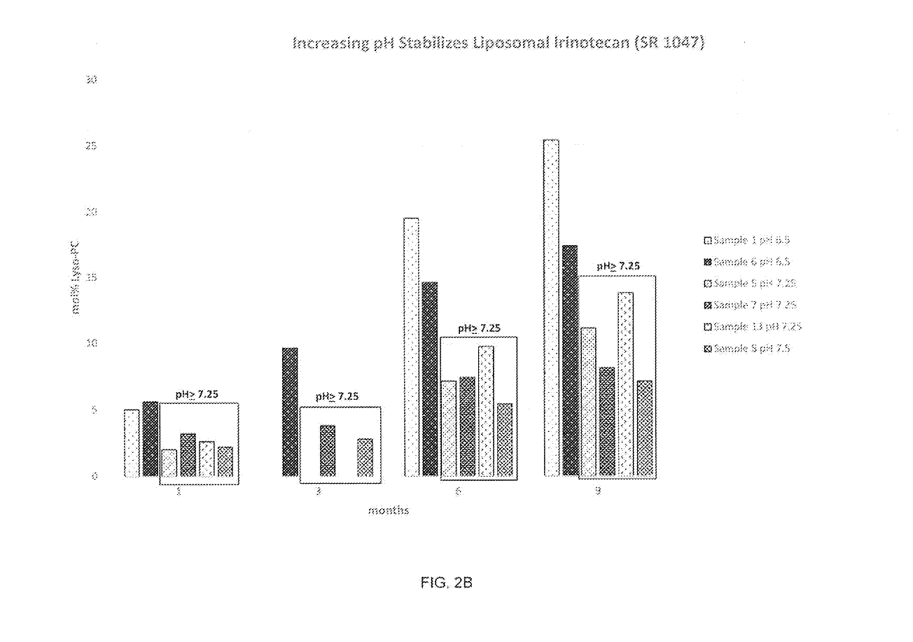

Increasing the pH of the irinotecan liposome preparations above 6.5 (e.g., 7.25 and 7.5) also decreased the amount of lyso-PC measured during refrigerated storage at 4° C. compared to irinotecan liposomes formed at comparable Stability Ratios. This trend was apparent at multiple concentrations of liposomal irinotecan compositions, including compositions prepared at a Stability Ratio of 785 (FIG. 2A) and 1047 (FIG. 2B) and compositions prepared with different concentrations of the irinotecan liposomes. For example, with respect to liposomal irinotecan compositions prepared at a strength of about 4.3 mg irinotecan moiety/mL, the mol % lyso-PC levels measured in Samples 5 and 7 were significantly lower at all data points (after the first 1, 6, and 9 months of storage at 4° C. after manufacturing) compared to the mol % lyso PC levels measured for Sample 1 at pH 6.5 (data in Table 1). Similarly, with respect to liposomal irinotecan compositions prepared at a strength of about 18.8 mg irinotecan moiety/mL, the mol % lyso-PC levels measured in Sample 13 (Table 4A) was significantly lower at all data points (after the first 1 and 9 months of storage at 4° C. after manufacturing) compared to the mol % lyso-PC levels measured for either Sample 12 or Sample 14 at pH 6.5 (data in Table 1). The liposomal irinotecan preparation of Sample 12 contained the irinotecan liposomes at a concentration providing a total of 4.3 mg irinotecan moiety per mL, while Sample 14 contained a higher concentration of irinotecan liposomes providing a total of about 18.8 mg irinotecan moiety per mL.

The pH of the composition comprising the irinotecan liposomes can be adjusted or otherwise selected to provide a desired storage stability property (e.g., to reduce formation of lyso-PC within the liposome during storage at 4° C. over 180 days), for example by preparing the composition at a pH of about 6.8-8.0 or any suitable pH value there between (including, e.g., 7.0-8.0, 7.2-7.5, 7.3, and 7.25). The final pH of the liposomal irinotecan preparation can be adjusted or otherwise selected after the loading of the trapping agent liposomes to form the irinotecan liposomes (e.g., with concomitant removal of the trapping agent cation to levels of about 100 ppm or less). In some embodiments, the pH of the liposomal irinotecan preparation after manufacturing and prior to storage is about any one of the following pH values: 6.8, 6.9, 7.0, 7.1, 7.2, 7.3, 7.4, 7.5, 7.6, 7.7, 7.8, 7.9, or 8.0. More preferably, the pH after manufacture and before storage is between 7.1 and 7.5 and even more preferably between about 7.2 and 7.3, and most preferably about 7.25. The pH can be adjusted by standard means, e.g. using 1N HCl or 1N NaOH, as appropriate. Preferably, the liposomal irinotecan composition has a pH greater than 6.5 (e.g., 7.0-7.5, including 7.25, 7.3, and 7.5). In some embodiments of the present invention, the pH of the liposomal irinotecan preparation after manufacture but prior to storage is above 6.5, preferably between to 7.2 and 7.3. In some embodiments of the present invention, the pH is from 7.2 to 7.5.

In some embodiments, the irinotecan liposomes are prepared as disclosed herein (e.g., according to Example 1, Example 6 or modifications or combinations thereof) with a Stability Ratio of about 950-1200 (e.g., 942-1130), and a final pH (prior to storage) of from 7.2 to 7.5 (including, e.g., 7.20, 7.25, 7.30, 7.35, 7.4, 7.45 or 7.50). The irinotecan and SOS trapping agent are preferably present in the liposome composition in an about 8:1 molar ratio. Preferably the Stability Ratio is 942-1130, the pH is about 7.25, and the irinotecan composition and SOS trapping agent are present in the liposome in an 8:1 molar ratio.

Stabilized irinotecan liposome preparations obtained by preferred processes with preferred Stability Ratios as defined herein can have about 80% less lyso-PC compared to irinotecan SOS liposomes prepared according to other processes having lower Stability Ratios. For example, stabilized liposomal irinotecan preparations with higher SR can have 80% less lyso-PC than observed in comparative Sample 12 (SR of 724) after 9 months of refrigerated storage. A (comparative) liposomal irinotecan of sample 12 was prepared with a Stability Ratio of about 724 by heating a lipid mixture having about a 3:2:0.015 mole ratio of 1,2-distearoyl-sn-glycero-3-phosphocholine (DSPC), cholesterol, and methoxy-terminated polyethylene glycol (MW 2000)-distearoylphosphatidyl ethanolamine (MPEG-2000-DSPE), in the presence of triethylamine (TEA) and sucrose octasulfate (“SOS” or “sucrosofate”) in a 8:1 mole ratio [(TEM₈SOS] at a sulfate group concentration of 0.65 M to generate TEA-SOS trapping agent liposomes. After removal of (TEM₈SOS not encapsulated in the TEA-SOS trapping agent liposomes, irinotecan was loaded into the resulting preparation containing the TEA-SOS trapping agent liposomes using a solution of irinotecan under conditions resulting in the release of TEA from the liposomes and loading into the liposomes a total amount of irinotecan moiety equivalent to that provided by 500 g (±10%) of irinotecan anhydrous free base per mole of phospholipids in the TEA-SOS trapping agent liposome preparation. The pH of the irinotecan liposome composition was 6.5 (measured in accordance with the subsection “pH Measurements” in the Examples section herein), with 4.3 mg of irinotecan moiety in the irinotecan liposomes per mL of the irinotecan liposome composition. These phosphatidylcholine-containing liposomal irinotecan compositions generated levels of lyso-PC in excess of 30 mol % (with respect to the total amount of phosphatidylcholine in the irinotecan liposome compositions) during 3 months (and over 35 mol % lyso-PC generated during 9 months) of refrigerated storage (2-8° C.).

The storage stability of irinotecan liposome preparations containing irinotecan SOS encapsulated in liposomes of about 100 nm (e.g., 100±20 nm) in diameter were obtained by methods disclosed herein were significantly increased in the resulting irinotecan liposomes where the Stability Ratio of the preparation method was greater than 942. Table 1, Table 4A and Table 4B provide data from a series of different irinotecan liposome preparations prepared according to the methods described in Example 1 and Example 2 (additional experimental details for preparation and characterization of each sample are included below in the Examples). By maintaining the constant drug loading ratio of the irinotecan moiety to total phospholipid, but varying the concentration of the SOS trapping agent, the effect of the Stability Ratio on the formation of lyso-PC in the liposome preparation was evaluated.

The results from these irinotecan liposome storage stability studies (including, e.g., data shown in the data in Tables 1 and 4A) demonstrated that reducing the concentration of SOS trapping agent (measured as the molar concentration of sulfate) used in the preparation of the liposomes, while keeping the ratio of irinotecan free base anhydrous (in g) to total phospholipid (in mol) approximately constant, resulted in greater storage stability of the irinotecan SOS liposomes, as measured by the amount of lyso-PC detected in the irinotecan liposome preparation after 6 and 9 months of refrigerated storage at 4° C. In liposome preparations with a pH of 6.5 after manufacture, but before storage (see “pH Measurements” method described herein), reducing the concentration of SOS trapping agent during liposome manufacture also lead to a reduction of amounts of lyso-PC detected in liposome preparations after storage at 4° C.

Without being bound by theory, it is believed that once purified from the extraliposomal trapping agent during preparation, the interior space of the liposome is acidified. This may be due to the redistribution of the amine component of the trapping agent salt from inside to the outside of the liposome following removal of extraliposomal TEA₈SOS, with a deposition of a hydrogen ion intraliposomally at each occurrence. Added drug, such as irinotecan, capable of protonation, also distributes between the exterior and the interior space of the liposome. Protonation of the drug distributed in the interior of the liposome and binding of the protonated drug to sucrosofate effects intraliposomal loading of drug and results in a reduction in the intraliposomal concentration of both TEA and hydrogen ions, decreasing the extent of intraliposomal acidification. In the case of irinotecan liposome it is postulated that at a drug load of 500 g irinotecan hydrochloride (i.e., 471 mg irinotecan moiety)/mol liposome phospholipid with SOS at a sulfate concentration of 0.6 M, there is incomplete exhaustion of the excess intraliposomal TEA.

The amount of lyso-PC measured in each of the irinotecan liposome preparations for Samples 1-12 is summarized in Table 1, arranged by increasing Stability Ratio used in preparing each sample by the process of Example 1. The mol % of lyso-PC was determined after storing the liposome preparations at 4° C. for 1, 3, 6, 9, and/or 12 months, as indicated in Table 1. For each sample, Table 1 provides the concentration of SOS used to prepare the liposome, expressed as molar concentration of sulfate groups (one molecule of SOS includes 8 sulfate groups). Table 1 also contains the stability ratio for each sample, calculated as the ratio of 471 g irinotecan moiety (based on the free base anhydrous) per mol phospholipid, divided by the concentration of sulfate groups in moles/L used to prepare the liposomes. The liposomes of the samples described in Table 1 each had a measured size (volume weighted mean) of between about 85-120 nm and an irinotecan encapsulation efficiency of at least 87.6%. Encapsulation efficiency was determined in accordance with subsection “Drug Retention and Stability.”

TABLE 1
Irinotecan Liposome Preparations with Various Stability Ratios and pH (liposome vesicles
formed from DSPC, cholesterol (Chol), and PEG-DSPE in a 3:2:0.015 molar ratio)^{c}

| | | Molar concentration | | | | | | |
|---|---|---|---|---|---|---|---|---|
| | | of sulfate groups | | | | | | |
| | | in the sucrosofate | | | | | | |
| | | entrapped in the | Stability | Time | Mol % | | | |
| Sample | pH | liposomes | Ratio | (months) | Lyso-PC | | Size | % SN38 |
| 12 | 6.5 | 0.65M | 724 | 0 | 3.8 | (±0.6) | 110.3 ± 19.7 | |
| | | | | 1 | 18.3 | (±1.2) | 120.1 ± 12.3 | 0.5 |
| | | | | 3 | 32.7 | (±1.9) | 107.6 ± 20.2 | 0.3 |
| | | | | 9 | 35.4 | (±0.5) | 101.2 ± 26.0 | 0.3 |
| | | | | 12 | 37.9 | | 106.4 ± 26.0 | 0.2 |
| 4 | 6.5 | 0.60M | 785 | 1 | 10.1 | (±0.3) | 107.6 ± 12.4 | 0.030 |
| | | | | 3 | | | 109.5 ± 13.0 | 0.014 |
| | | | | 6 | 30.2 | (±0.9) | 105.3 ± 17.7 | 0 |
| | | | | 9 | 35.8 | (±0.6) | 105.7 ± 27.9 | 0.005 |
| 9 | 6.5 | 0.60M | 785 | 1 | 11.3 | (±0.8) | 107.6 ± 26.6 | |
| | | | | 3 | 22.1 | (±1.3) | 108.6 ± 13.4 | 0.016 |
| | | | | 6 | 29.8 | (±1.9) | 112.6 ± 9.4 | 0.010 |
| | | | | 9 | 34.7 | (±1.2) | 111.1 ± 15.2 | 0.005 |
| 10 | 7.25 | 0.6M | 785 | 1 | 9.6 | (±0.8) | 98.9 ± 7.0 | |
| | | | | 3 | 16.9 | (±1.1) | 108.4 ± 11.8 | 0.011 |
| | | | | 6 | 24.1 | (±0.8) | 103.0 ± 8.9 | 0.010 |
| | | | | 9 | 29.0 | (±0.6) | 105.9 ± 23.8 | 0.005 |
| 11 | 7.5 | 0.60M | 785 | 1 | 9.33 | (±0.5) | 102.2 ± 23.6 | |
| | | | | 3 | 17.1 | (±5.01) | 102.6 ± 9.8 | 0.012 |
| | | | | 6 | 22.8 | (±0.7) | 105.9 ± 18.1 | 0.010 |
| | | | | 9 | 28.7 | (±3.1) | 112.4 ± 15.3 | 0.005 |
| 3 | 6.5 | 0.50M | 942 | 1 | 9.9 | (±0.2) | 109.7 ± 13.7 | 0.024 |
| | | | | 3 | | | 104.7 ± 12.6 | 0.014 |
| | | | | 6 | 26.5 | (±0.3) | 106.6 ± 12.7 | 0 |
| | | | | 9 | 35.7 | (±0.6) | 88.5 ± 36.5 | 0.006 |
| 2 | 6.5 | 0.475M | 992 | 1 | 5.7 | (±0.2) | 89.4 ± 31.9 | 0.028 |
| | | | | 3 | | | 84.9 ± 33.8 | 0.018 |
| | | | | 6 | 17.0 | (±0.4) | 93.4 ± 26.0 | |
| | | | | 9 | 23.6 | (±1.0) | 102.6 ± 18.8 | 0.006 |
| 1 | 6.5 | 0.45M | 1047 | 1 | 5.0 | (±0.1) | 108.5 ± 13.6 | 0.036 |
| | | | | 3 | | | 98.6 ± 31.3 | 0.022 |
| | | | | 6 | 19.5 | (±0.6) | 112.6 ± 11.4 | 0 |
| | | | | 9 | 25.4 | (±0.6) | 93.8 ± 27.90 | 0 |
| 6 | 6.5 | 0.45M | 1047 | 1 | 5.6 | (±1.37) | 106.7 ± 18.2 | |
| | | | | 3 | 9.6 | (±1.4) | 96.4 ± 26.0 | 0.051 |
| | | | | 6 | 14.6 | (±0.5) | 98.2 ± 24.0 | 0.01 |
| | | | | 9 | 17.4 | (±0.4) | 109.2 ± 12.6 | 0.006 |
| 5 | 7.25 | 0.45M | 1047 | 1 | 2.0 | (±0.3) | 106.4 ± 18.5 | 0.033 |
| | | | | 3 | | | 103.9 ± 18.8 | 0.015 |
| | | | | 6 | 7.1 | (±0.4) | 107.2 ± 17.3 | 0 |
| | | | | 9 | 11.1 | (±0.1) | 100.0 ± 28.1 | 0.007 |
| 7 | 7.25 | 0.45M | 1047 | 1 | 3.2 | (±0.3) | 105.3 ± 13.1 | |
| | | | | 3 | 3.8 | (±0.5) | 104.1 ± 16.7 | 0.022 |
| | | | | 6 | 7.4 | (±0.5) | 105.5 ± 13.4 | 0.010 |
| | | | | 9 | 8.1 | (±0.7) | 107.3 ± 13.0 | 0.006 |
| 8 | 7.5 | 0.45M | 1047 | 1 | 2.2 | (±0.1) | 102.8 ± 14.2 | |
| | | | | 3 | 2.8 | (±0.1) | 103.5 ± 11.1 | 0.018 |
| | | | | 6 | 5.4 | (±0.2) | 91.8 ± 28.6 | 0.010 |
| | | | | 9 | 7.1 | (±1.2) | 108.2 ± 19.0 | 0.006 |

^{c}Measured according to Method B, as described herein.

As described in Example 1, each of the irinotecan liposome preparation samples in Table 1 was formed from a mixture of DSPC, cholesterol, and MPEG-2000-DSPE having about a 3:2:0.015 molar ratio and then loaded with irinotecan at a ratio of about 471 g irinotecan moiety (derived from irinotecan or a salt thereof—for example providing an amount of irinotecan moiety equivalent to about 500 g of irinotecan HCl anhydrous) per mole phospholipid. Each irinotecan liposome preparation contained different amounts of the SOS trapping agent and were formulated at different pH values, as indicated in Table 1. The amount of lyso-PC was measured in each irinotecan liposome preparation using the TLC method (Method B) in the Examples. The amount of lyso-PC was determined for each sample at various times, including a measurement of all samples after 9 months of continuous refrigerated storage (at 4° C.). All samples in Table 1 were made by using a protonated TEA counter-ion for SOS (i.e., loading irinotecan into liposomes encapsulating various concentrations of TEA₈SOS, as specified in Table 1).

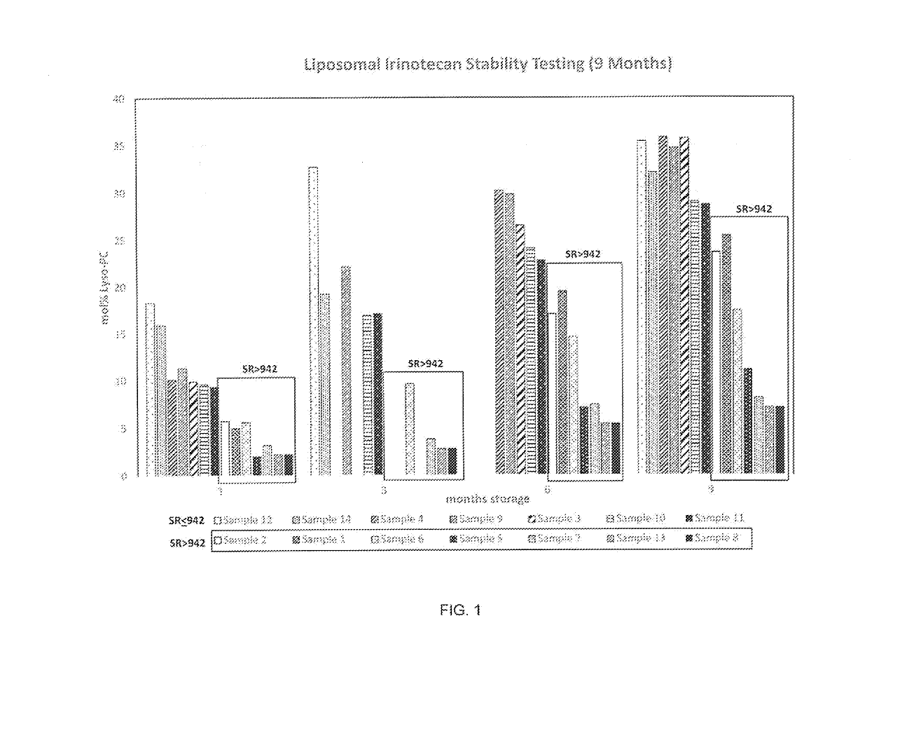

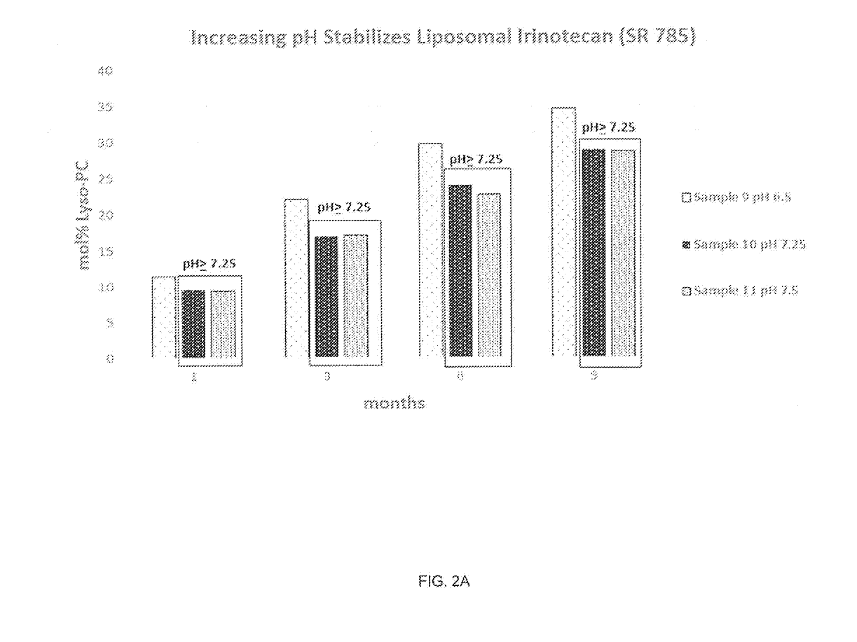

FIG. 1 is a graph showing the mol % of lyso-PC in irinotecan liposome preparations from Table 1 (Samples 1-12) and Table 4A (Samples 13-14). Lyso-PC was determined after storage of each sample measured using the TLC of Method B below for storage at 4° C. for 1, 3, 6, and/or 9 months. The greatest amount of lyso-PC formation was observed in samples prepared with Stability Ratios of 725-942 at pH 6.5 (Samples 3, 4, 9, 12 and 14). Less lyso-PC formation was observed in samples prepared with a higher pH at a Stability Ratio of 785 (Samples 10 and 11). However, significantly less lyso-PC formation was observed in samples prepared at higher Stability Ratios of 992-1047, at pH 6.5 (Samples 1, 2, and 6), at pH 7.25 (Samples 5, 7, and 13). The least amount of lyso-PC formation was observed in Sample 8 (prepared with Stability Ratio of 1047, with a pH of 7.5).

Referring to data in Table 1, the irinotecan liposomes of samples 1-11 retained good colloidal stability up to 9 months at 4° C., as judged by the absence of precipitation and the relatively narrow and reproducible particle size distributions, where the irinotecan moiety concentration corresponded to 4.71 mg/mL irinotecan free base anhydrous. Irinotecan was efficiently and stably entrapped with minimal leakage (<10%) over extended periods of storage (see “Drug Retention and Stability” method described herein). Samples 1 and 2 had identical initial loads of about 471 g irinotecan moiety (as explained above, based on the free base anhydrous) per mole phospholipid, but lower SOS concentrations of 0.45 M sulfate groups and 0.475 M sulfate groups, respectively. Similarly, samples 6, 7, and 8 had a lower SOS concentration of 0.45 M sulfate but the same drug load of 471 g irinotecan moiety (as explained above, based on the free base anhydrous)/mol phospholipid, resulting in a considerably lower lyso-lipid content (7-17% after 9 months).

Increased levels of lyso-PC were measured in samples at pH of 6.5 regardless of the drug load or trapping agent concentrations during liposome manufacture, reaching up to as high as 35.7 mol % lyso-PC for some samples after 9 months storage at 4° C. (1, and 3). Adjustment of pH to 7.25 rendered the liposomes less susceptible to lyso-PC formation. Thus, sample 13 had lyso-PC levels after 6 months at 4° C. of 9.72 mol %, while sample 1 had about twice that level (i.e., 19.5 mol %). Samples with higher drug to trapping agent concentration ratios and higher pH formed less lyso-PC, as seen in samples 7 and 8 having 7-8 mol % lyso-PC after 9 months. The combination of a higher drug trapping agent ratio and higher pH (e.g., compared to Sample 12) reduced lyso-lipid formation. The most stable liposome formulation combines the higher drug/trapping agent ratios (i.e. Stability Ratios above 942 with the higher external pH above 6.5).

Furthermore, the % SN38 measured in the irinotecan liposome preparations 1-11 over 9 months was not greater than about 0.05% SN38 (i.e., relative amount of SN38 by comparison to irinotecan and SN38), while sample 12 irinotecan liposome preparation had from 0.20-0.50% SN38 measured over the same time period (determined by “Drug Analysis” method described herein). In each of samples 1-5 and 13, irinotecan was stably entrapped with low leakage from liposomes (less than 13%; determined by “Drug Retention and Stability” method described herein) and low conversion to the active cytotoxic SN-38, less than 0.1%, and in samples stored at higher pH (7.25), less than 0.05%.

Table 2 provides data selected from Table 1, a summary of the amount of mol % lyso-PC detected in the irinotecan liposome preparations in Table 1 formulated at the same pH as the (comparative) Sample 12 (6.5), but at different concentrations of SOS trapping agent (i.e., at different Stability Ratios).

TABLE 2
Irinotecan Liposome Stability Ratio and Lyso-PC (after 9 months at 4° C., pH 6.5)

| | | | 4 | 5 | | |
|---|---|---|---|---|---|---|
| | | | % SR | % lyso-PC @ | 6 | 7 |
| | 2 | 3 | increase | 9 mos | Incremental | Incremental |
| 1 | Stability | Lyso-PC @ | relative to | relative to | % SR | % lyso-PC @ |
| Sample | Ratio | 9 mos | Sample 12 | Sample 12 | increase | 9 months |
| 12 | 724 | 35.4 | 0 | 0 | 0 | 0 |
| 9 | 785 | 34.7 | +8.3 | −2 | +8 | −2 |
| 3 | 942 | 35.7 | +30 | +1 | +20 | +3 |
| 2 | 992 | 23.6 | +37 | −33 | +5 | −34 |
| 6 | 1047 | 17.4 | +44 | −51 | +6 | −26 |
| 1 | 1047 | 25.4 | +44 | −28 | +6 | +8 |

Significant reductions of lyso-PC observed in samples during storage at 4° C. were achieved by preparing irinotecan liposome compositions at pH 6.5 having a Stability Ratio above 950 (e.g., 950-1050). The data in Table 1 illustrates that preparing irinotecan liposomes with a Stability Ratio of greater than 942 containing a SOS trapping agent and irinotecan results in a subsequent reduction in the formation of lyso-PC in the liposomal irinotecan preparation during refrigerated storage. Reducing the amount of SOS trapping agent (i.e., increasing the Stability Ratio) by up to 30% relative to the Sample 12 irinotecan liposome preparation resulted in a slight increase in the amount of lyso-PC by about 1% after 9 months of refrigerated storage. However, increasing the amount of SOS trapping agent in an irinotecan liposome preparation having a Stability Ratio of above 942 results in a significant and unexpected decline in the amount of lyso-PC (mol %) present after 9 months of refrigerated storage at 4° C. For example, a subsequent 5% incremental increase in the Stability Ratio above 942 (i.e., a Stability Ratio of 992 in Sample 2) resulted in a dramatic decrease of the amount of lyso-PC (mol %) present by 34%, compared to Sample 3, equivalent to a 33% decrease in the amount of lyso-PC (mol %) compared to Sample 12 (as measured at 9 months of refrigerated storage at 4° C.). Overall, after 9 months of refrigerated storage at 4° C., reductions of lyso-PC (mol %) of about 28-51% can be achieved by raising the Stability Ratio of irinotecan liposome above 942, compared to Sample 12. In some embodiments, the irinotecan SOS liposome compositions have a Stability Ratio of above 942. In preferred embodiments, the irinotecan SOS liposome preparations have a Stability Ratio of 942-1130 or greater (e.g., Stability Ratios of 992-1047). Accordingly, preferred irinotecan SOS liposome compositions have a Stability Ratio of above 950, preferably above 1000, including irinotecan SOS liposome preparations with a Stability Ratio of 1000-1200 or greater (e.g., Stability Ratios of 1053-1111).

In some embodiments of the present invention, the stability of an irinotecan liposome preparation containing irinotecan SOS encapsulated in liposomes is significantly increased by raising the pH of the preparation after manufacture but prior to storage above pH 6.5. By selecting a desired drug loading ratio of the irinotecan moiety (based on the free base anhydrous) per mol phospholipid (e.g., 471 g/mol liposome PL in Example 1 or 500 g/mol liposome PL in Example 3), but varying the pH of the final pH of the irinotecan liposome composition, the effect of the pH on the formation of lyso-PC in the liposome preparation was evaluated. In some embodiments, irinotecan liposome preparations above pH 6.5 (e.g., preferably 7.25 or 7.0) are prepared with a Stability Ratio of greater than 942 (preferably greater than 950, and most preferably greater than 992, most preferably about 990-1,200), with reduced formation of intraliposomal lyso-PC during refrigerated storage compared to liposomal irinotecan prepared at the same pH but at a Stability Ratio of 942 or less.

FIG. 2A shows the mol % lyso-PC measured in liposomal irinotecan samples in Table 1, all prepared at a Stability Ratio of 785. Increasing the pH of the liposomal irinotecan preparation from 6.5 (Sample 9) to 7.25 (Sample 10) to 7.5 (Sample 11) resulted in progressively lower lyso-PC formation during stability storage at 4° C. A similar effect is observed in liposomal irinotecan preparations prepared at higher Stability Ratios. FIG. 2B shows the mol % lyso-PC measured in liposomal irinotecan samples, all prepared at a Stability Ratio of 1047. Increasing the pH of the liposomal irinotecan preparation from 6.5 (Samples 1 and 6) to 7.25 (Samples 5, 7 and 13) to 7.5 (Sample 8) resulted in progressively lower lyso-PC formation during stability storage at 4° C.

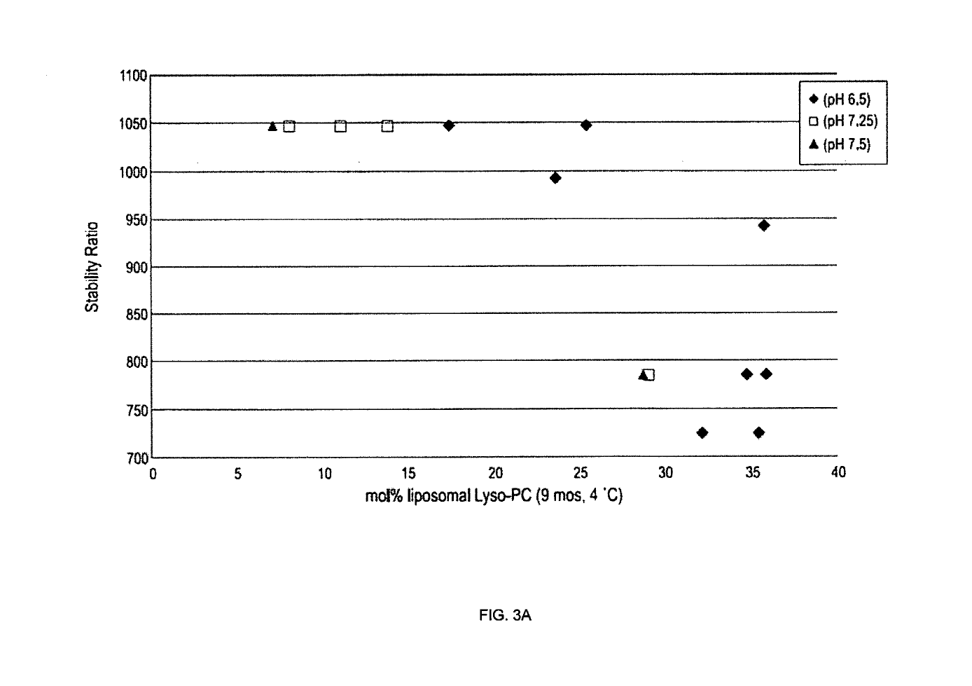

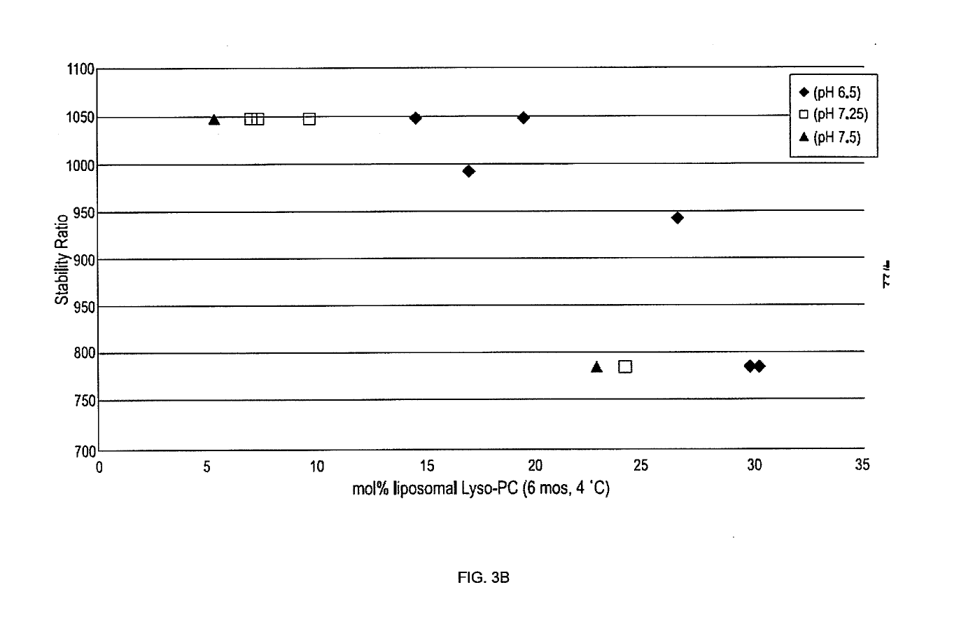

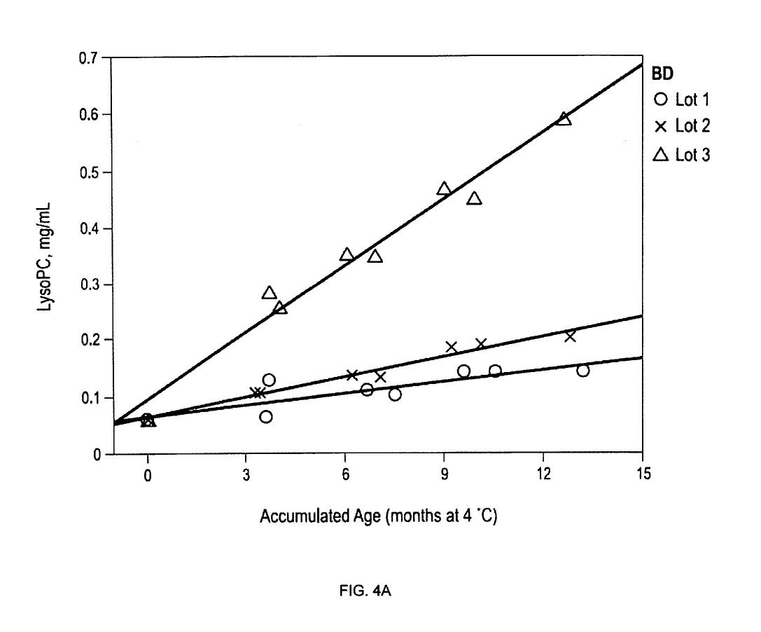

FIG. 3A shows a plot depicting the mol % lyso-PC measured in each sample in Table 1 and Table 4A after 9 months of storage at 4° C. Samples having both a Stability Ratio greater than about 900 and a pH of greater than 6.5 (e.g., 7.25 and 7.5) contained less than 20 mol % lyso-PC after 9 months of refrigerated storage at 4° C. FIG. 3B is a graph of the Stability Ratio values versus the mol % lyso-PC of irinotecan liposome compositions after 6 months of storage at 4° C. (data in Table 1). The data points indicated with diamonds correspond to irinotecan samples having a pH of 6.5, measured after manufacture but prior to storage. Samples having both a Stability Ratio greater than about 950 and a pH of greater than 6.5 (e.g., 7.25 and 7.5) contained less than 10 mol % lyso-PC after 6 months of refrigerated storage at 4° C. Samples having both a Stability Ratio greater than about 950 and a pH of 6.5 contained less than 20 mol % lyso-PC after 6 months of refrigerated storage at 4° C.

The formulation of liposomes encapsulating irinotecan can be an injectable formulation containing liposomes (including injectable formulations that can be subsequently diluted with a pharmaceutically acceptable diluent prior to administration to a patient. Stabilized liposomal irinotecan pharmaceutical compositions can include high-density (e.g., concentrated) irinotecan liposome preparations containing irinotecan or a salt thereof at an irinotecan moiety concentration equivalent to that provided by from 4.5 to 5.5 mg/mL irinotecan hydrochloride trihydrate (i.e., 3.9-4.8 mg/mL irinotecan free base anhydrous). The irinotecan liposome preparations can be diluted prior to administration to a patient (e.g., with a pharmaceutically acceptable sterile diluent such as 500 mL of 0.9% sodium chloride solution or 5% dextrose injectable solution). The injectable liposomal irinotecan preparations can contain DSPC at a concentration of from 6.13 to 7.49 mg/mL (preferably about 6.81 mg/mL), cholesterol at a concentration of from 2-2.4 mg/mL (preferably about 2.22 mg/mL), and a PEG-DSPE copolymer (e.g., a PEG-DSPE co-polymer, or a MPEG-DSPE copolymer such as MPEG-2000-DSPE) at a concentration of 0.11-0.13 mg/mL (preferably about 0.12 mg/mL). The stabilized liposomal irinotecan composition can have low amounts of lyso-PC (e.g., less than about 0.5 mg/mL lyso-PC in a dosage form such as a sterile product vial, after manufacturing), and can form low amounts of lyso-PC (e.g., forming less than about 1.5 mg/mL lyso-PC during refrigerated storage at 2-8° C. for at least 6-36 months including about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months after manufacturing). The liposomal irinotecan product can also provide suitable amounts of the irinotecan, preferably in a more potent lactone form. The present invention includes pharmaceutical irinotecan liposome compositions that can be stored refrigerated (i.e., at 2-8° C.) for at least the first 6 months, preferably at least the first 9 months, following manufacture without the containing or forming levels of lyso-PC above 20 mol %. More preferably, the present invention provides for compositions containing an amount of irinotecan moiety equivalent to that provided by between 4.7-5.3 mg/mL irinotecan hydrochloride trihydrate (i.e., 4.1-4.6 mg irinotecan moiety free anhydrous base) (the irinotecan can be present as a sucrose octasulfate salt encapsulated within the liposomes), along with (DSPC) at about 6-8 mg/mL, preferably about 6.5-7.5 mg/mL (including any other ranges and amounts between 6-8 mg/mL), cholesterol at about 2-2.5 mg/mL (e.g., 2.1-2.4 and other ranges and amounts between 2-2.5 mg/mL), and a PEG-DSPE co-polymer (such as MPEG-DSPE and PEG-DSPE co-polymers including MPEG-2000-DSPE) at about 0.1-0.2 mg/mL (including any ranges and amounts between 0.1 and 0.2 mg/mL, such as about 0.15) mg/mL, wherein the composition contains (or generates) no more than 20 mol % lyso-PC at 6 or even at 9 months when stored at 2-8° C., or no more than 2 mg/mL lyso-PC at 21 months when stored at 2-8° C.

In a preferred embodiment, the liposomal irinotecan composition contains irinotecan or a salt thereof in an irinotecan moiety concentration equivalent to that provided by 4.3 mg/mL irinotecan free anhydrous base per mL, while also containing less than about 1 mg/mL (or less than about 20 mol %) lyso-PC after 6 months of refrigerated storage at about 4° C. In a preferred embodiment, the liposomal irinotecan composition of the present invention contains irinotecan or a salt thereof in an irinotecan moiety concentration equivalent to that provide by 4.3 mg/mL irinotecan free anhydrous base per mL, while also containing less than about 2 mg/mL (or less than about 30 mol %) lyso-PC at 12 months of refrigerated storage 2-8° C., even more preferably at about 4° C.

In some embodiments, the concentration of the irinotecan moiety equivalent to that provided by the irinotecan free anhydrous base in the liposome preparation is about 2.5, about 3.0, about 3.5, about 4.0, about 4.3, about 4.5, about 5.0, about 5.5, or about 6.0 mg/mL. In some embodiments, the concentration of the irinotecan moiety, equivalent to that provided by the irinotecan free anhydrous base in the liposome preparation, is 2.5-3.5, 3.5-4.5, 4.5-5.5, or 5.5-6.5 mg/mL. Most preferably it is 4.5-5.5 mg/mL. The liposomal irinotecan preparation can be a vial containing about 43 mg irinotecan free anhydrous base in the liposomal irinotecan preparation having a volume of about 10 mL, which can be subsequently diluted (e.g., into 500 mL of a pharmaceutically acceptable diluent) prior to intravenous administration to a patient.

The liposomal irinotecan compositions—including irinotecan liposomes and other compositions and preparations disclosed herein of the invention can be used in therapy and methods of treatment, and or in the preparation of medicaments for the treatment of disease, such as cancer. In some embodiments, a therapy comprises administration of a liposomal irinotecan composition for the treatment of cancer. For example the cancer is selected from the group consisting of small cell lung cancer (SCLC), pancreatic cancer (e.g., metastatic adenocarcinoma of the pancreas with disease progression following gemcitabine-based therapy, or previously-untreated pancreatic cancer), colorectal cancer, biliary tract cancer, breast cancer, gastric cancer, cervical cancer, and ovarian cancer. In some embodiments the cancer is pancreatic cancer, optionally adenocarcinoma of the pancreas, such as metastatic adenocarcinoma of the pancreas, for example where disease progression has occurred following gemcitabine-based therapy. In some embodiments the cancer is ovarian cancer. In some embodiments the cancer is small cell lung cancer. In some embodiments, the cancer is biliary tract cancer.

The liposome composition may be used in a treatment regimen alone or with one or more other compounds or compositions. For example, liposomal irinotecan can be administered as a mono-therapy once every two weeks (e.g., at a 90 mg/m² dose based on the amount of irinotecan moiety administered) (i.e., in the absence of other anti-neoplastic agents) to treat a patient diagnosed with small cell lung cancer (SCLC). The administration of the liposomal irinotecan compositions in combination with one or more other compounds or compositions may be simultaneous, separate or sequential. The one or more other compounds or compositions may be further therapeutics, e.g. further anticancer agents, or may be compounds which are designed to ameliorate the negative side effects of the therapeutic agents. In some embodiments, the liposome composition is administered with leucovorin. In some embodiments, the liposome composition is administered with 5-fluorouracil (5-FU). In some embodiments, the liposome composition is administered with leucovorin (LV) and 5-fluorouracil (5-FU). The 5-FU can be administered once every two weeks at a dose of 2400 mg/m², and leucovorin can be administered at a dose of 200 mg/m² (1 form) or 400 mg/m² (1+d racemic form) (e.g. in a 28-day antineoplastic treatment cycle). In some embodiments, the liposomal irinotecan composition is administered in a treatment regimen without gemcitabine (e.g., for the treatment of pancreatic cancer). In some embodiments where the liposome composition is used to treat ovarian cancer, the liposomal irinotecan composition is administered with a PARP (poly ADP ribose polymerase) inhibitor.

In some embodiments, liposomal irinotecan composition is an irinotecan SOS liposome preparation formulated for intraparenchymal administration to a patient during a convection enhanced delivery therapy. The concentration of the irinotecan moiety, equivalent to that provided by the irinotecan free anhydrous base in the final liposome preparation is about 17, about 20, about 25, about 30, about 35, or about 40 mg/mL. In some embodiments, the concentration of the irinotecan moiety, equivalent to that provided by the irinotecan free anhydrous base in the final liposome preparation is 17-20, 17-25, 17-30, 17-35, or 17-40 mg/mL. Most preferably, the total concentration of the irinotecan moiety, equivalent to that provided by the irinotecan free anhydrous base (e.g., as irinotecan sucrose octasulfate) in the irinotecan liposome preparation is 17 mg/mL, or 35 mg/mL. The liposome preparation can be in a sterile container enclosing irinotecan sucrose octasulfate liposomes in the liposome preparation at an irinotecan moiety concentration equivalent to that provided by about 17 mg/mL or about 35 mg/mL or about 17-35 mg/ml irinotecan free anhydrous base for local administration to a patient (e.g., into the brain of a patient diagnosed with a glioma, to a location within the brain as part of a convection enhanced delivery therapy). The about 17-35 mg irinotecan/mL (e.g., 17.2-34.4 mg/ml) concentration of irinotecan liposomes can be equivalently expressed as the amount of irinotecan free anhydrous base present in about 20-40 mg (e.g., 20-40 mg/ml) of irinotecan hydrochloride trihydrate, per mL of the irinotecan liposome preparation. For example, the liposomal irinotecan preparation can be administered into the brain of a patient (e.g., via one or more catheters surgically placed in an intra-tumoral location) at doses providing a total of irinotecan moiety equivalent to that provided by 17 mg, 26 mg, 52 mg, or 70 mg total irinotecan free anhydrous base. The irinotecan total volume of the irinotecan liposome preparation delivered into the intra-tumoral location within the brain of the patient can be about 1-2 mL (e.g., 1.0, 1.5, or 2.0 mL) over a period of about 2-4 hours (e.g., 2-3 hours, 3-4 hours, or 2-4 hours). In some embodiments, the liposomes are prepared as described in one or more Examples or other embodiments herein, but the concentration of the final liposome composition is increased so that the formulation contains an irinotecan moiety concentration equivalent to irinotecan hydrochloride trihydrate at a concentration of about 10, 15, 20, 25, 30, 35, 40, 45, or 50 mg/mL (e.g., for administration to a tumor site in the brain, including convection enhanced delivery). In some embodiments, the irinotecan moiety concentration is equivalent to that provided by irinotecan hydrochloride trihydrate between 5-10, 10-20, 20-30, 30-40 or 40-50 mg/mL. In some embodiments, the liposome compositions mentioned under this section are used to treat brain tumor or any other condition in a mammal, as described U.S. Pat. No. 8,658,203, which is incorporated herein by reference in its entirety.

The irinotecan liposomes preferably contain irinotecan sucrosofate encapsulated within a vesicle formed from lipids comprising a lecithin and cholesterol in a 3:2 molar ratio. The vesicle can also contain a polyethylene-glycol (PEG) derivatized phospholipid, such as MPEG-2000-DSPE. The amount of MPEG-2000-DSPE can be less than 1 mol % of the liposome lipid (e.g., about 0.3 mol. % in a vesicle consisting of DSPC, cholesterol and MPEG-2000-DSPE in a 3:2:0.015 molar ratio). The PEG can be distributed on both the inside and the outside of the liposome lipid vesicle enclosing the irinotecan. The encapsulated irinotecan is preferably in the form of a salt with sulfate ester of sucrose (sucrosofate), such as irinotecan sucrosofate (CAS Registry Number 1361317-83-0). Preferably, at least 95% and most preferably at least about 98% of the irinotecan in the irinotecan liposome composition is encapsulated within a liposome vesicle, with a total irinotecan moiety concentration of about 3.87-4.73 mg irinotecan (free anhydrous base) per mL of the irinotecan liposome composition. The pH of the irinotecan liposome composition is preferably about 6.5-8.0 outside the liposome, or about 6.6-8.0, 6.7-8.0, 6.8-8.0, 6.9-8.0, or 7.0-8.0, and preferably about 7.2-7.6. In some embodiments, the pH is about 7.2-7.5. In some embodiments, the pH is about 7.25. In other embodiments, the pH is about 7.25-7.5. In other embodiments, the pH is about 7.4-7.5.

Preferably, the liposomal irinotecan compositions, when administered to humans at a dose corresponding to 70 mg/m² of irinotecan free base once every two weeks, are characterized by the following pharmacokinetic properties: a Cmax 37.2 (8.8) μg irinotecan (as free base anhydrous)/mL and AUC_{0-∞} 1364 (1048) h·μg irinotecan/mL (for irinotecan); or (for SN-38), Cmax 5.4 (3.4) μg SN-38 (as free base anhydrous)/mL; AUC_{0-∞} 620 (329) h·ng SN-38/mL.

One preferred example of a storage stable irinotecan liposome preparation is the product marketed as ONIVYDE® (irinotecan liposome injection) (Merrimack Pharmaceuticals, Inc., Cambridge, Mass.). The ONIVYDE® product is a topoisomerase-1 inhibitor, formulated with irinotecan hydrochloride trihydrate into a liposomal dispersion, for intravenous use. The ONIVYDE® product is indicated, in combination with fluorouracil and leucovorin, for the treatment of patients with metastatic adenocarcinoma of the pancreas after disease progression following gemcitabine-based therapy. The recommended dose of the ONIVYDE® product for the treatment of post-gemcitabine metastatic pancreatic cancer is 70 mg/m² (irinotecan free base) administered by intravenous infusion over 90 minutes once every 2 weeks, administered in combination with leucovorin and fluorouracil. The recommended starting dose of the ONIVYDE® product in these pancreatic cancer patients known to be homozygous for the UGT1A1*28 allele is 50 mg/m² administered by intravenous infusion over 90 minutes. Increase the dose of the ONIVYDE® product to 70 mg/m² as tolerated in subsequent cycles. There is no recommended dose of the ONIVYDE® product for patients with serum bilirubin above the upper limit of normal.

The ONIVYDE® product is administered to patients as follows. First, the calculated volume of the ONIVYDE® product is withdrawn from the vial. This amount of the ONIVYDE® product is then diluted in 500 mL 5% Dextrose Injection, USP or 0.9% Sodium Chloride Injection, USP and mixed by gentle inversion. The dilution should be protected from light. The dilution is then administered within 4 hours of preparation when stored at room temperature or within 24 hours of preparation when stored under refrigerated conditions [2° C. to 8° C. (36° F. to 46° F.)]. The diluted solution is allowed to come to room temperature prior to administration, and it should not be frozen. The dilution is then infused over 90 minutes without the use of in-line filters, and the unused portion is discarded.

The ONIVYDE® product is formulated with irinotecan hydrochloride trihydrate, a topoisomerase inhibitor, into a liposomal dispersion for intravenous use. The chemical name of irinotecan hydrochloride trihydrate is (S)-4,11-diethyl-3,4,12,14-tetrahydro-4-hydroxy-3,14-dioxo1H-pyrano[3′,4′:6,7]-indolizino[1,2-b]quinolin-9-yl-[1,4′bipiperidine]-1′-carboxylate, monohydrochloride, trihydrate. The empirical formula is C₃₃H₃₈N₄O₆.HCl.3H2O and the molecular weight is 677.19 g/mole.

The ONIVYDE® product is provided as a sterile, white to slightly yellow opaque isotonic liposomal dispersion. Each 10 mL single-dose vial contains the equivalent of 43 mg irinotecan free base at a concentration of 4.3 mg/mL irinotecan free base anhydrous per mL (i.e., 4.3 mg irinotecan moiety/mL). The liposome is a unilamellar lipid bilayer vesicle, approximately 110 nm in diameter, which encapsulates an aqueous space containing irinotecan in a gelated or precipitated state as the sucrose octasulfate salt. The vesicle is composed of 1,2-distearoyl-sn-glycero-3-phosphocholine (DSPC) 6.81 mg/mL, cholesterol 2.22 mg/mL, and methoxy-terminated polyethylene glycol (MW 2000)-distearoylphosphatidyl ethanolamine (MPEG-2000-DSPE) 0.12 mg/mL. Each mL also contains 2-[4-(2-hydroxyethyl) piperazin-1-yl]ethanesulfonic acid (HEPES) as a buffer 4.05 mg/mL and sodium chloride as an isotonicity reagent 8.42 mg/mL.

Irinotecan liposome injection is a topoisomerase 1 inhibitor encapsulated in a lipid bilayer vesicle or liposome. Topoisomerase 1 relieves torsional strain in DNA by inducing single-strand breaks. Irinotecan and its active metabolite SN-38 bind reversibly to the topoisomerase 1-DNA complex and prevent re-ligation of the single-strand breaks, leading to exposure time-dependent double-strand DNA damage and cell death. In mice bearing human tumor xenografts, irinotecan liposome administered at irinotecan HCl-equivalent doses 5-fold lower than irinotecan HCl achieved similar intratumoral exposure of SN-38.

The plasma pharmacokinetics of total irinotecan and total SN-38 were evaluated in patients with cancer who received the ONIVYDE® product, as a single agent or as part of combination chemotherapy, at doses between 50 and 155 mg/m², and 353 patients with cancer using population pharmacokinetic analysis.

The pharmacokinetic parameters of total irinotecan and total SN-38 following the administration of the ONIVYDE® product at 70 mg/m² as a single agent or part of combination chemotherapy are presented below.

TABLE 3
Summary of Mean (±Standard Deviation) Total Irinotecan and Total SN-38

| | Total Irinotecan | | | | | Total SN-38 | | |
|---|---|---|---|---|---|---|---|---|
| | Cₘₐₓ | AUC_{0-∞} | t_{1/2} | CL | V_{d} | Cₘₐₓ | AUC_{0-∞} | t_{1/2} |
| Dose | [μg/mL] | [h · μg/mL] | [h] | [L/h] | [L] | [ng/mL] | [h · ng/mL] | [h] |
| (mg/m²) | (n = 25) | (n = 23) | (n = 23) | (n = 23) | (n = 23) | (n = 25) | (n = 13) | (n = 13) |
| 70 | 37.2 | 1364 | 25.8 | 0.20 | 4.1 | 5.4 | 620 | 67.8 |
| | (8.8) | (1048) | (15.7) | (0.17) | (1.5) | (3.4) | (329) | (44.5) |

Cₘₐₓ: Maximum plasma concentration
AUC_{0-∞}: Area under the plasma concentration curve extrapolated to time infinity
t_{1/2}: Terminal elimination half-life
CL: Clearance
V_{d}: Volume of distribution

Over the dose range of 50 to 155 mg/m², the Cmax and AUC of total irinotecan increases with dose. Additionally, the Cmax of total SN-38 increases proportionally with dose; however, the AUC of total SN-38 increases less than proportionally with dose.

Direct measurement of irinotecan liposome showed that 95% of irinotecan remains liposome-encapsulated, and the ratios between total and encapsulated forms did not change with time from 0 to 169.5 hours post-dose.

The ONIVYDE® product should be stored at 2° C. to 8° C. (36° F. to 46° F.), should be protected from light, and should not be frozen.

### EXAMPLES

The synthesis and characterization of several irinotecan liposome preparations is described in the following Examples. Unless otherwise indicated in the Examples, these irinotecan liposomes can be obtained by the following multi-step processes disclosed herein. The invention therefore also provides methods of making irinotecan liposomes in line with the preparative methods set out in this subsection and in the Examples, and variations and combinations thereof. Unless otherwise indicated, all ranges disclosed herein are inclusive of the endpoints, and the endpoints are independently combinable with each other.

First, liposome-forming lipids are dissolved in heated ethanol. These lipids included DSPC, cholesterol, and MPEG-2000-DSPE. Unless otherwise indicated, the DSPC, cholesterol, and MPEG-2000-DSPE are present in about a 3:2:0.015 molar ratio. The resulting ethanol-lipid composition is dispersed in an aqueous medium containing substituted ammonium and polyanion under conditions effective to form a properly sized (e.g. 80-120 nm or 95-115 nm etc.), essentially unilamellar liposomes containing the substituted ammonium ion and polyanion trapping agent (SOS). The liposome dispersion can be formed, e.g., by mixing the ethanolic lipid solution with the aqueous solution containing a substituted ammonium ion and polyanion at the temperature above the lipid transition temperature, e.g., 60-70° C., and extruding the resulting lipid suspension (multilamellar liposomes) under pressure through one or more track-etched, e.g. polycarbonate, membrane filters with defined pore size, e.g. 50 nm, 80 nm, 100 nm, or 200 nm. Preferably the substituted ammonium is a protonated triethylamine (TEA) or diethylamine (DEA) and the polyanion is sucrose octasulfate (SOS), preferably combined in a stoichiometric ratio (e.g., TEA₈SOS). The concentration of the TEA₈SOS can be selected based on the amount of irinotecan loaded into the liposomes (e.g., to substantially or completely exhaust the concentration loading gradient across the liposome, and/or provide a liposome containing SOS and irinotecan in about a 1:8 mole ratio). For example, to prepare irinotecan SOS liposomes with 471 g or 500 g irinotecan moiety/mol phospholipid, the TEA₈SOS used preferably has a concentration of about 0.4-0.5 M sulfate groups (e.g. 0.45 M or 0.475 M of sulfate groups). All or substantially all non-entrapped TEA or SOS is then removed (e.g., by gel-filtration, dialysis, or ultrafiltration/diafiltration).

The resulting trapping agent liposomes (e.g., encapsulating substituted ammonium compound such as TEA₈SOS or DEA₈SOS) are then contacted with an irinotecan solution under conditions effective to load the irinotecan into the trapping agent liposomes (i.e., conditions that allow the irinotecan to enter the liposome in exchange with TEA leaving the liposome). The irinotecan loading solution (e.g. at 15 mg/ml of anhydrous irinotecan-HCl, which can be prepared using corresponding amounts of irinotecan-HCl trihydrate) preferably contains an osmotic agent (e.g., 5% dextrose) and a pH of 6.5 (unless otherwise stated, pH values are mentioned in this specification were determined at room temperature). Drug loading is facilitated by increase of the temperature of the composition above the transition temperature of the liposome lipids (e.g., to 60-70° C.) to accelerate the transmembrane exchange of substituted ammonium compound (e.g., TEA) and irinotecan. In some embodiments, the irinotecan sucrosofate within the liposome is in a gelated or precipitated state.

The loading of irinotecan by exchange with substituted ammonium compound (e.g., TEA or DEA) across the liposome is preferably continued until all or substantially all of the substituted ammonium compound (e.g., TEA) is removed from the liposome, thereby exhausting all or substantially all of the concentration gradient across the liposome. Preferably, the irinotecan liposome loading process continues until the gram-equivalent ratio of irinotecan to SOS is at least 0.9, at least 0.95, 0.98, 0.99, or 1.0 (or ranges from about 0.9-1.0, 0.95-1.0, 0.98-1.0, or 0.99-1.0). Preferably, the irinotecan liposome loading process continues until at least 90%, at least 95%, at least 98%, or at least 99%, or more of the TEA is removed from the liposome interior. In some embodiments of the present invention, the irinotecan SOS liposome composition prepared in this manner using TEA₈SOS contain less than 100 ppm TEA. In some embodiments of the present invention, the irinotecan SOS liposome composition prepared in this manner using TEA₈SOS contain 20-100 ppm, 20-80 ppm, 40-80 ppm, or 40-100 ppm TEA. Some embodiments contain less than 30 ppm TEA.

Extra-liposomal irinotecan and substituted ammonium compound (e.g., TEA or DEA) can be removed to obtain the final irinotecan liposome product. This removal can be facilitated by a variety of methods, non-limiting examples of which include gel (size exclusion) chromatography, dialysis, ion exchange, and ultrafiltration/diafiltration methods. The liposome external medium is replaced with injectable, pharmacologically acceptable fluid, e.g., buffered (pH between 7.1 to 7.5, preferably pH between 7.2 and 7.3) isotonic saline. Finally, the liposome composition is sterilized, e.g., by 0.2-micron filtration, dispensed into single dose vials, labeled and stored, e.g., upon refrigeration at 2-8° C., until use. The liposome external medium can be replaced with pharmacologically acceptable fluid at the same time as the remaining extra-liposomal irinotecan and ammonium/substituted ammonium ion (e.g., TEA) is removed.

### Quantification of Trapping Agent

For the purpose of the present invention, unless otherwise indicated, the liposomal trapping agent and substituted ammonium compound counter-ion (e.g., TEA₈SOS) is quantified based on the concentrations used for preparing the liposomes and calculated based on the number sulfate groups of the trapping agent. For example, a 0.1 M TEA₈SOS would be expressed herein as 0.8 M/L sulfate because each molecule of SOS has eight sulfate groups. In cases where a different trapping agent is used, this calculation would be adjusted, depending on the number of anionic groups (e.g., sulfate groups) per molecule of trapping agent.

### Quantification of Lyso-PC in Irinotecan Liposome Preparations

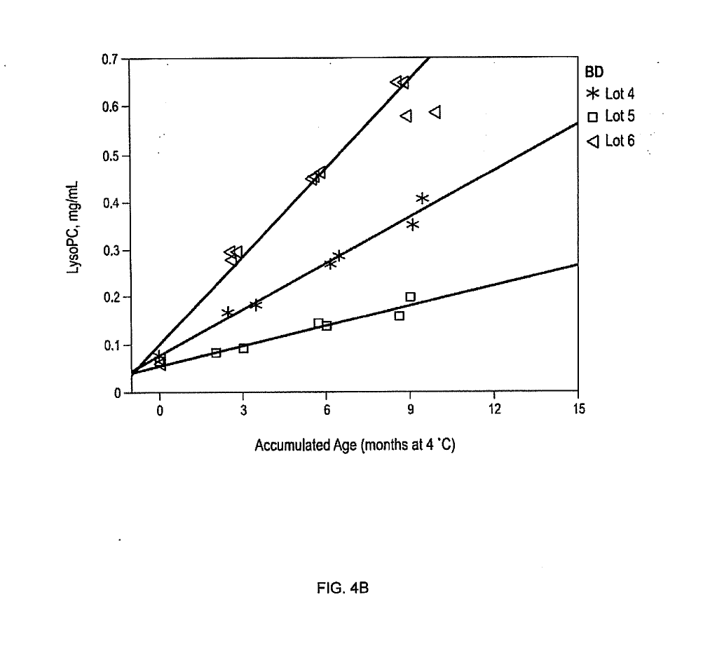

The amount of lyso-PC (mg/mL) in the irinotecan sucrose octasulfate liposome preparations tested to obtain data in FIGS. 4A, 4B, 5, 9A, 9B and 9C was obtained by the HPLC method (“Method A”), which is described in Example 8.

A different preparative (TLC) method (herein, “Method B”) was used obtain the lyso-PC measurements for data in FIGS. 1, 2A, 2B, 3A and 3B, and Example 7 (including from Samples 1-23 herein), where the lyso-phospholipid (mol %) was determined by the following TLC method followed by phosphate analysis, rather than the HPLC method (Method A) discussed immediately above. The following steps were followed to measure lyso-PC by Method B. An aliquot of liposome sample containing approximately 500 nmol phospholipid (PL) (e.g. 0.05 mL of a 10 mM PL liposome solution) was desalted using a PD-10 column (GE Healthcare) equilibrated with water. The sample is eluted from the column with water and divided into three portions containing approximately 150 nmol of PL each, then dried under vacuum using a centrifugal concentrator (Savant Speed Vac Concentrator, Model#SVC100X). The dried lipids were dissolved in 30 μl of chloroform/methanol (5/1, vol/vol) and applied to the non-adsorbent region of a normal phase silica gel TLC plate (Uniplate by Analtech, cat #44921) using a glass syringe. The TLC was run with a mobile phase consisting of chloroform/methanol/30% ammonium hydroxide/water (60/40/2.5/3.75, v/v/v/v) and the lipid visualized using iodine vapor. Determination of the PL was conducted by scraping the spots corresponding to phospholipid and lyso-phospholipid on the TLC into separate 12×75 mm borosilicate tubes for subsequent phosphate analysis.

The quantification of molar amounts of liposomally co-encapsulated irinotecan and sulfate compound is provided in the Examples.

### Materials

For preparing samples 1-5 and 13 in Example 1 and samples 12 and 14-18 in Example 2, USP GMP grade irinotecan hydrochloride ((+)-7-ethyl-10-hydroxycamptothecine 10-[1,4′-bipiperidine]-1′-carboxylate, monohydrochloride, trihydrate, CAS Reg. No. 100286-90-6) was purchased from SinoPharm (Taipei, Taiwan); 1,2-distearoyl-sn-glycero-3-phosphocholine (DSPC) and methoxy-terminated polyethylene glycol (MW-2000)-distearoylphosphatidylethanolamine ((MPEG-2000-DSPE) were purchased from Avanti Polar Lipids (Alabaster, Ala., USA); ultrapure cholesterol (Chol) was obtained from Calbiochem (La Jolla, Calif., USA); and sucrose octasulfate was obtained from Euticals (Lodi, Italy).

For preparing samples 6-11 in Example 1, irinotecan hydrochloride trihydrate was obtained from PharmaEngine (Taiwan); 1,2-distearoyl-sn-glycero-3-phosphocholine (DSPC) and methoxy-terminated polyethylene glycol (MW-2000)-distearoylphosphatidylethanolamine ((MPEG-2000-DSPE) were purchased from Avanti Polar Lipids (Alabaster, Ala., USA); ultrapure cholesterol (Chol) was obtained from Calbiochem (La Jolla, Calif., USA); and sucrose octasulfate was obtained from Euticals (Lodi, Italy).

For preparing samples 19-23 in Example 7, Vinorelbine (VNB) was obtained from the pharmacy as a solution of vinorelbine tartate 10 mg/mL (Glaxo-SmithKline), and topotecan (TPT) powder was obtained as a gift from Taiwan Liposome Company (Taipei, Taiwan).

All other chemicals, of analytical or better purity, were obtained from common suppliers.

Methods: The following methods were used in preparing Samples 1-5 and 13 and Samples 6-11 and 19-23, and Samples 12, and 14-18, to the extent not indicated otherwise below.

### Triethylammonium Sucrose Octasulfate Preparation

Triethylammonium sucrose octasulfate (TEA₈SOS) and diethylammonium sucrose octasulfate (DEA₈SOS) were prepared from the sodium salt of sucrose octasulfate using ion exchange chromatography. Briefly, 15 g of sucrose octasulfate (sodium salt) was dissolved in water to give a sulfate concentration of 2.64 M. A Dowex 50W-8X-200 cation exchange resin was employed to prepare the acidic form of sucrose octasulfate. Defined resin was washed twice with 2 vol of 1 N NaOH, then with ddH₂O (doubly distilled water) to neutral pH, washed twice with 2 vol of 1 N HCl, and finally washed to neutral with ddH₂O and then repeated. A column was poured to a volume of 450 mL of resin and washed with 3 vol of 3 N HCl, and then rinsed with ddH₂O until the conductivity reaches less than 1 μS/cm. The sucrose octasulfate (sodium salt) solution (approximately 10% of column capacity) was loaded on the column and eluted with ddH₂O. The column eluent was monitored using a conductivity detector to detect the elution of the sucrose octasulfate from the column. The acidic sucrose octasulfate was then titrated with triethylamine or diethylamine to a pH in between 6-7, and the sulfate content determined using a method modified from B. Sorbo et al., Methods in Enzymology, 143: 3-6, 1984 (see Sulfate Determination). The solution was finally diluted to a sulfate concentration corresponding to 0.65 M sulfate. The pH was typically in the range of 6-7. Residual sodium was determined using a sodium electrode, and any solution with residual sodium above 1 mol-% was not utilized further.

### Sulfate Group Determination

Sulfate content in the sucrose octasulfate solutions was determined with a turbidimetric-based assay. Solutions consist of: (1) 15 g PEG 6000 and 1.02 g barium acetate in 100 mL water; (2) 142 mg sodium sulfate in 1 mL water; (3) Barium working solution: add dropwise 0.1 mL of the sodium sulfate solution to 100 mL barium solution while stirring. This solution should equilibrate for 1 hour before use and can be stored no longer than one week; (4) 0.4 M trisodium citrate solution; (118 mg trisodium citrate/mL water); and (5) sulfate standard at 10 mM diluted in water from 1 N sulfuric acid. Using borosilicate test tubes the standards and solutions were made to a final volume of 100 μl. The standards were made in the range of 0.2-1 μmol sulfate (20-100 μl of the 10 mM standard). For samples of 0.6 M sulfate solution, a dilution of 1/100 and volume of 100 μl (0.6 μmol) was used. Each 100 μl sample/standard was treated with 100 μl of 70% perchloric acid and heated at 110-120° C. for 12 minutes. After cooling, 0.8 mL of the 0.4 M trisodium citrate solution was added followed by vortexing. A 0.25 mL volume from a stirring barium working solution was transferred to each tube and vortexed immediately. All samples/standards were allowed to equilibrate for 1 hour followed by vortexing and measurement of the absorbance at 600 nm. A linear standard curve of SO₄ concentrations versus OD600 was used to determine unknown SO₄ concentrations.

### Sucrose Octasulfate Determination by HPLC

The concentration of sucrose octasulfate (mg/mL) in a sample can be calculated based on the area of the sucrose octasulfate peak produced from a standard of known concentration. The calculated concentration of the sucrose octasulfate is then used to calculate the concentration of sulfate (mM) in a sample.

The sample to be analyzed is chromatographed by HPLC using a Phenomenex, Bondclone 10μ NH₂, 300×3.90 mm, PN 00H-3128-CO, or Waters pondapak NH₂ 10 μm 125 Å, (3.9 mm×300 mm), Part No. WAT084040 using a mobile phase of 0.60 M ammonium sulfate, pH 3.0 eluted at 1.00 mL/min at a column temperature of 40° C. Samples are detected by a refractive index detector, which is also at 4 0° C., for example, using an Agilent HPLC with Refractive Index Detector. USP Potassium Sucrose Octasulfate heptahydrate is used as a reference standard; CAS 76578-81-9, CAT No. 1551150.

The SOS assay standard and assay control samples are integrated using a baseline to baseline integration. The TEA-SOS samples are then integrated using a baseline to baseline integration. This may be performed manually beginning the baseline before the void volume valley to the end of the SOS tail, then dropping a line at the start of the TEA peak and the low point between the two peaks. Note: If a single baseline beginning before the void volume valley to the end of the SOS tail crosses the low-point between TEA and SOS peaks, two separate lines may be used that will approximate the baseline to baseline approach. TEA-SOS samples will show a TEA peak at a relative retention time of approximately 0.45 to the retention time of the SOS peak.

### Drug Analysis

HPLC analysis of irinotecan was conducted on a Dionex system using a C₁₈ reverse phase silica column (Supelco C₁₈ column, 250 mm×4 mm inner diameter, particle size of 5 μm) preceded by a Supelco C₁₈ guard column. A sample injection volume of 50 μl was used, and the column was eluted isocratically at a flow rate of 1.0 mL/min with a mobile phase consisting of 0.21 M aqueous triethylammonium acetate pH 5.5 and acetonitrile (73:27, v:v). Irinotecan and SN-38 typically eluted in 5.1 min and 7.7 min respectively. Irinotecan was detected by absorbance at 375 nm using a diode array detector, and SN-38 was detected by fluorescence (370 nm excitation and 535 nm emission).

### Phosphate Determination

The following phosphate determination method was used for analyzing Samples 1-23. A modified Bartlett phosphate assay can be used to measure phospholipid (PL). Standards ranging from 10-40 nmol of phosphate were placed in 12×75 mm borosilicate tubes and treated exactly as the samples. Sulfuric acid (100 μl of 6 M H₂O₄) was added to each tube placed in a heating block and heated to 180° C. for 45 minutes. Hydrogen peroxide (20 μl of a 30% solution) was added to each tube and then heated at 150° C. for 30 minutes. Ammonium molybdate (0.95 mL of a 2.2 g/l solution) and ascorbic acid (50 μl of a 10% aqueous solution) were subsequently added to each tube. After vortexing, the tubes were developed in boiling water for 15 minutes and then cooled to room temperature. For lysolipid analysis using thin-layer chromatography (TLC), the silica was pelleted by centrifugation at 1000 rpm for 5 minutes, and the blue color was measured in the supernatant by reading the absorbance at 823 nm. Samples not containing silica can eliminate the centrifugation step.

### Drug Retention and Stability

Liposomal irinotecan stability (in terms of drug retention) was determined by separating the liposomal irinotecan from extraliposomal irinotecan using PD-10 (Sephadex G-25) size exclusion columns. Drug leakage was determined by comparison of the irinotecan (HPLC) to PL (described in Phospholipid Determination) ratio before and after separation of the extraliposomal irinotecan. Degradation of the irinotecan was determined by observation of additional peaks in the chromatogram after HPLC analysis. The irinotecan-to-phospholipid ratios and the drug encapsulation efficiencies are calculated using formulas 1 and 2 below, respectively.

$$\begin{matrix}Irinotecan \text{-} to \text{-} phospholipid ratio ( g Irinotecan \text{/} mol PL ) = \frac{[ Irinotecan ] ( mg \text{/} mL ) * 1000}{[ phospholipid ] ( mM )} & ( 1 ) \\ Encapsulation efficiency ( \% ) = \frac{( Irinotecan \text{-} to \text{-} phospholipid ratio ) AC}{( Irinotecan \text{-} to \text{-} phospholipid ratio ) BC} & ( 2 )\end{matrix}$$

where (Irinotecan-to-phospholipid ratio) AC is the drug to phospholipid ratio after purification on the G-25 size exclusion column and (Irinotecan-to-phospholipid ratio) BC is the drug-to-phospholipid ratio before purification on the column.

### Determination of Encapsulated and Free Irinotecan in Liposomal Compositions

Liposomally encapsulated and free (non-encapsulated) irinotecan in the irinotecan sucrosofate liposomal compositions of Examples 3 and 4 was determined using a cartridge adsorption method. Oasis 60 mg 3 cc HLB cartridges (Waters) were conditioned by sequential passage of 2 mL methanol, 1 mL HEPES-buffered saline (HBS; 5 mM HEPES, 140 mM NaCl, pH 6.5), and 0.5 mL of 10% human serum albumin in normal saline, followed by 1 mL of HBS. Liposomal irinotecan sucrosofate compositions were diluted with normal saline to about 2.2 mg/mL irinotecan, and 0.5 mL aliquots were applied on the cartridges. The eluate was collected, the cartridges were rinsed with two portions of HBS (1.5 mL, 3 mL), and the rinses combined with the eluate to make a liposome fraction. The cartridges were additionally rinsed with 1.5 mL HBS and eluted with two 3-mL portions of methanol-HCl (90 vol. % methanol, 10 vol. % 18 mM HCl). The eluates were combined to make the free drug fraction. Liposomal drug fractions were transferred into 25-mL volumetric flasks, and free drug fractions were transferred into 10-mL volumetric flasks, brought to the mark with methanol-HCl, mixed well, and the liposome fraction flasks were heated for 10 minutes at 60° C. to solubilize the drug. Upon cooling, the solutions were filtered, and irinotecan was quantified in both fractions using reverse phase HPLC on a Phenomenex Luna C18(2) column, isocratically eluted with 20 mM potassium phosphate pH 3.0 methanol mixture (60:40 by volume) with UV detection at 254 nm. The drug peaks were integrated, and the amount of irinotecan in the samples was calculated by comparison to the linear standard curve obtained under the same conditions using irinotecan hydrochloride trihydrate USP reference standard. The drug encapsulation ratio was calculated as a percentage of encapsulated drug relative to the total of free and encapsulated drug in the sample.

### pH Measurements

The pH was always measured at ambient temperature (i.e., 20-25° C.) using a potentiometric standard glass electrode method. The pH of liposome formulations was measured accordingly by putting the glass electrode into the liposome formulation and obtaining a pH reading.

### Analysis of Samples for TEA/DEA Ppm

Samples analysis was performed by headspace gas chromatographic (GC) separation utilizing gradient temperature elution on a capillary GC column (50 m×0.32 mm×5 μm Restek Rtx-5 (5% phenyl-95% dimethylpolysiloxane)) followed by flame ionization detection (FID). A sample preparation and a standard preparation were analyzed, and the resulting peak area responses were compared. The amount of residual amine (e.g., TEA or diethyl amine (DEA)) was quantitated using external standards. In the case of TEA, the standard was >99%. Other reagents include Triethylene glycol (TEG), sodium hydroxide, and deionized (DI) water.

GC conditions were: carrier gas: helium; column flow: 20 cm/sec (1.24 mL/min); split ratio: 10:1 (which can be adjusted as long as all system suitability criteria are met); injection mode: split 10:1; liner: 2 mm straight slot (recommended but not required); injection port temperature: 140° C., detector temperature: 260° C. (FID); initial column oven temperature: 40° C.; column oven temperature program:

| rate (° C./min) | temperature (° C.) | hold time (min) |
|---|---|---|
| n/a | 400 | 0 |
| 2 | 100 | 0 |
| 20 | 240 | 17 |

54 min Runtime

Headspace Parameters: platen temperature: 90° C.; sample loop temperature: 100° C.; transfer line temperature: 100° C.; equilibration time: 60 minutes; injection time: 1 minute; vial pressure: 10 psi; pressurization time: 0.2 minute; shake: on (medium); injection volume: 1.0 mL of headspace; GC Cycle Time: 60 minutes (recommended but not required).

If no TEA is detected, report as “none detected;” if TEA results are <30 ppm, report as <QL (30 ppm); of TEA results are ≥30 ppm, report to a whole number.

### Determination of Liposome Size

Liposome particle size was measured using dynamic light scattering (DLS) using a Malvern ZetaSizer Nano ZS™ or similar instrument in aqueous buffer (e.g., 10 mM NaCl, pH 6.7) at 23-25° C. using the method of cumulants. The z-average particle size and the polydistersity index (PDI) were recorded. The instrument performance was verified using Nanosphere NIST traceable standard of 100 nm polymer (Thermo Scientific 3000 Series Nanosphere Size Standard P/N 3100A, or equivalent with a certificate of analysis that includes Hydrodynamic Diameter). As used herein, “DLS” refers to dynamic light scattering and “BDP” refers to bulk drug product.

### Example 1: Effects of SOS Trapping Agent Concentration and pH on Liposomal Irinotecan Preparation Storage Stability

The aim of this study was to determine, among other things, any changes in the physical and chemical stability of liposomes encapsulating irinotecan and sucrose octasulfate (SOS) trapping agent when stored at about 4° C. for certain periods of time. For this study, the liposomal concentration of the SOS trapping agent was reduced, while the ratio of 471 g irinotecan moiety per total mols of phospholipid was maintained. Data obtained from Example 1 is provided in Table 1.

A series of irinotecan SOS liposome preparations were prepared in a multistep process using different concentrations of SOS trapping agent and adjusting the pH of the final liposomal preparation to different pH values. Each of the irinotecan SOS liposome preparations contained irinotecan moiety concentration equivalent to 4.7 mg/mL irinotecan hydrochloride trihydrate. Irinotecan SOS liposome preparations of Samples 1-5 and 13 were prepared by a multi-step process of Example 1.

DSPC, cholesterol (Chol), and PEG-DSPE were weighed out in amounts that corresponded to a 3:2:0.015 molar ratio, respectively (e.g., 1264 mg/412.5 mg/22.44 mg). The lipids were dissolved in chloroform/methanol (4/1, v/v), mixed thoroughly, and divided into 4 aliquots (A-D). Each sample was evaporated to dryness using a rotary evaporator at 60° C. Residual chloroform was removed from the lipids by placing under vacuum (180 μtorr) at room temperature for 12 hours. The dried lipids were dissolved in ethanol at 60° C., and pre-warmed TEA₈SOS of appropriate concentration was added so that the final alcohol content was 10% (v/v). The lipid concentration was approximately 75 mM. The lipid dispersion was extruded at about 65° C. through 2 stacked 0.1 um polycarbonate membranes (Nuclepore™) 10 times using Lipex thermobarrel extruder (Northern Lipids, Canada), to produce liposomes with a typical average diameter of 95-115 nm (determined by quasi-elastic light scattering; see subsection “Determination of Liposome Size”). The pH of the extruded liposomes was adjusted as needed to correct for the changes in pH during the extrusion. The liposomes were purified by a combination of ion-exchange chromatography and size-exclusion chromatography. First, Dowex™ IRA 910 resin was treated with 1 N NaOH, followed by 3 washes with deionized water, and then followed by 3 washes of 3 N HCl, and then multiple washes with water. The liposomes were passed through the prepared resin, and the conductivity of the eluted fractions was measured by using a flow-cell conductivity meter (Pharmacia, Uppsala, Sweden). The fractions were deemed acceptable for further purification if the conductivity was less than 15 μS/cm. The liposome eluate was then applied to a Sephadex G-75 (Pharmacia) column equilibrated with deionized water, and the collected liposome fraction was measured for conductivity (typically less than 1 μS/cm). Cross-membrane isotonicity was achieved by addition of 40% dextrose solution to a final concentration of 5% (w/w) and the buffer (Hepes) added from a stock solution (0.5 M, pH 6.5) to a final concentration of 10 mM.

A stock solution of irinotecan was prepared by dissolving irinotecan.HCl trihydrate powder in deionized water to 15 mg/mL of anhydrous irinotecan-HCl, taking into account water content and levels of impurities obtained from the certificate of analysis of each batch. Drug loading was initiated by adding irinotecan in an amount of 500 g irinotecan HCl anhydrous (corresponding to 471 g irinotecan free base anhydrous) per mol liposome phospholipid and heating to 60±0.1° C. for 30 minutes in a hot water bath. The solutions were rapidly cooled upon removal from the water bath by immersing in ice cold water. Extraliposomal drug was removed by size exclusion chromatography, using Sephadex G75 columns equilibrated and eluted with Hepes buffered saline (10 mM Hepes, 145 mM NaCl, pH 6.5). The samples were analyzed for irinotecan by HPLC and phosphate by the method of Bartlett (see subsection “Phosphate Determination”). For storage, the samples were divided into 4 mL aliquots, and the pH was adjusted using 1 N HCl or 1 N NaOH, sterile filtered under aseptic conditions, and filled into sterile clear glass vials that were sealed under argon with a Teflon® lined threaded cap and placed in a thermostatically controlled refrigerator at 4° C. At defined time points, an aliquot was removed from each sample and tested for appearance, liposome size, drug/lipid ratio, and drug and lipid chemical stability.

With respect to Example 1, liposome size distribution was determined in the diluted samples by dynamic light scattering using Coulter Nano-Sizer at 90 degree angle and presented as Mean±Standard deviation (nm) obtained by the method of cumulants.

Irinotecan liposome preparations of samples 1-5 and 13 were further obtained as follows. The freshly extruded liposomes comprised two groups each incorporating TEA₈SOS as the trapping agent at the concentrations of (A) 0.45 M sulfate group (112.0±16 nm), (B) 0.475 M sulfate group (105.0±16 nm), (C) 0.5 M sulfate group (97±30 nm), and (D) 0.6 M sulfate group (113±10 nm). Samples 1-5 and 13 were loaded at an initial ratio of 471 g irinotecan free base anhydrous per mol total phospholipids and purified as described above in the Example 1 description (equivalent to 500 g irinotecan HCl anhydrous). Samples 1, 5 and 13 were derived from extruded sample (A); sample 2 was from extruded sample (B); samples 3 and 4 were from extruded samples (C) and (D), respectively. Following purification, pH adjustment was made using 1 N HCl or 1 N NaOH prior to sterilization and the filling of the vials. Data from samples 1-5 are shown in Table 1 (Example 1), and data from sample 13 is shown in Table 4A (Example 2).

Irinotecan liposome preparations of samples 6-11 were further obtained as follows. The freshly extruded liposomes comprised two groups each incorporating TEA₈SOS as the trapping agent at the concentrations of (A) 0.45 M sulfate group (116±10 nm) and (B) 0.6 M sulfate group (115.0±9.0 nm). Samples 6-8 were derived from extruded sample (A), and samples 9-11 were from extruded sample (B). Following purification, pH adjustment was made if necessary by addition of 1 N HCl or 1 N NaOH as appropriate. Sample 12 was prepared as described in Example 2 and is included in Table 1 for comparative purposes.

### Example 2: Increasing Concentration of Irinotecan Liposomes in a Liquid Preparation

The aim of this storage stability study was to determine any changes in the physical and chemical stability of liposomal irinotecan SOS when stored at 4° C. During this study, the concentration of the sucrose octasulfate (SOS) trapping agent used for liposome preparation was kept at a sulfate group concentration of 0.65 M, while varying: (1) the initial counter ion of the SOS trapping agent during the preparation of the irinotecan liposomes (using TEA₈SOS or DEA₈SOS), (2) the ratio of the amount of irinotecan free base anhydrous (in gram) to phospholipid (in mol) (about 471 g or 707 g irinotecan moiety (as explained above, based on the free base anhydrous) per mole phospholipid), (3) the concentration of the irinotecan free base anhydrous in the liquid irinotecan preparation (4.7 mg/mL or 18.8 mg/mL encapsulated irinotecan (based on the equivalent concentration of irinotecan moiety from irinotecan hydrochloride trihydrate) in the liquid irinotecan liposome preparation), (4) the pH to which the irinotecan liposome preparation was adjusted (pH 6.5, or 7.25), and (5) the buffer of the irinotecan liposome preparation (HEPES or histidine).

The formulation parameters investigated include: liposome size, drug to phospholipid ratio in the irinotecan liposomes, the irinotecan drug encapsulation efficiency and general appearance, the presence of irinotecan degradation products, and lyso-PC (in mol %) formation.

A series of irinotecan SOS liposome preparations were prepared in a multistep process using different concentrations of SOS trapping agent relative to encapsulated irinotecan and adjusting the pH of the final liposomal preparation to different pH values. DSPC, cholesterol (Chol), and PEG-DSPE were weighed out in amounts that corresponded to a 3:2:0.015 molar ratio, respectively (730.9 mg/238.5 mg/13.0 mg). The lipids were dissolved in chloroform/methanol (4/1, v/v), mixed thoroughly, and divided into 2 aliquots. Each sample was evaporated to dryness using a rotary evaporator at 60° C. Residual chloroform was removed from the lipids by placing under vacuum (180 μtorr) at room temperature for 12 hours. The dried lipids were dissolved in ethanol at 60° C., and pre-warmed TEA₈SOS or DEA₈SOS (at a concentration of 0.65 M sulfate group) was added so that the final alcohol content was 10% (v/v) and the samples were designated A and B, respectively. The lipid concentration was approximately 75 mM. The lipid dispersion was extruded through 0.1 μm polycarbonate membranes (Nuclepore™) 10 times, to produce liposomes with a typical average diameter of 95-115 nm. The pH of the extruded liposomes was adjusted as needed (with 1 N NaOH) to the selected preparation pH. The liposomes were purified by a combination of ion-exchange chromatography and size-exclusion chromatography. First, Dowex™ IRA 910 resin was treated with 1 N NaOH, followed by 3 washes with deionized water, and then followed by 3 washes of 3 N HCl, and then multiple washes with water. The conductivity of the eluted fractions was measured by using a flow-cell conductivity meter (Pharmacia, Uppsala, Sweden). The fractions were deemed acceptable for further purification if the conductivity was less than 15 μS/cm. The liposome eluate was then applied to a Sephadex G-75 (Pharmacia) column equilibrated with deionized water, and the collected liposome fraction was measured for conductivity (typically less than 1 μS/cm). Cross-membrane isotonicity was achieved by addition of 40% dextrose solution to a final concentration of 5% (w/w), and the buffer (Hepes) was added from a stock solution (0.5 M, pH 6.5) to a final concentration of 10 mM.

A stock solution of irinotecan was prepared by dissolving 326.8 mg irinotecan.HCl trihydrate powder in 20.0 mL deionized water to 15 mg/mL of anhydrous irinotecan-HCl, taking into account water content and levels of impurities obtained from the certificate of analysis of each batch. Drug loading was initiated by adding irinotecan free base anhydrous at 500 g/mol or 750 g/mol phospholipid and heating to 60±0.1° C. for 30 min in a hot water bath. The solutions were rapidly cooled upon removal from the water bath by immersing in ice cold water. Extraliposomal drug was removed by size exclusion chromatography, using Sephadex G75 columns equilibrated and eluted with Hepes buffered saline (10 mM) (HBS), pH 6.5 for sample A and histidine buffered saline at pH 7.25 for sample B. The samples were analyzed for irinotecan by HPLC and phosphate by the method of Bartlett (see Phosphate Determination).

For storage, the samples were divided into 4 mL aliquots, and the pH was adjusted if necessary using 1 N HCl or 1 N NaOH, sterile filtered under aseptic conditions, and filled into sterile clear glass vials that were sealed under argon with a Teflon® lined threaded cap, and placed in a thermostatically controlled refrigerator at 4° C. At defined time points, an aliquot was removed from each sample and tested for appearance, size, drug/lipid ratio, and drug and lipid chemical stability.

The liposome size was determined in the diluted samples by dynamic light scattering using Coulter Nano-Sizer at 90 degree angle and presented as Mean±Standard deviation (nm) obtained by the method of cumulants.

The results from comparative stability studies are provided in Table 4A (for samples prepared using TEA₈SOS trapping agent starting material) and Table 4B (for samples prepared using DEA₈SOS trapping agent starting material).

TABLE 4A
Irinotecan Liposomes prepared with TEA₈SOS Trapping Agent in Hepes Buffer (10 mM)^{d}

| | | | Molar concentration | | | | | |
|---|---|---|---|---|---|---|---|---|
| | | | of sulfate groups | | | | | |
| | Final | [irinotecan]/ | in the sucrosofate | | | | | |
| | Prep | total mol | entrapped in the | Stability | [irinotecan] | Time | Mol % | |
| Sample | pH | PL | liposomes | ratio | g/mol | (months) | Lyso-PC | |
| 12 | 6.5 | 471 | 0.65M | 724 | 4.7 | 0 | 3.8 | (±0.6) |
| | | | | | | 1 | 18.3 | (±1.2) |
| | | | | | | 3 | 32.7 | (±1.9) |
| | | | | | | 9 | 35.4 | (±0.5) |
| | | | | | | 12 | 37.9 | (±0.5) |
| 13 | 7.25 | 471 | 0.45M | 1047 | 18.8 | 1 | 2.6 | (±0.6) |
| | | | | | | 6 | 9.72 | (±1.9) |
| | | | | | | 9 | 13.8 | (±1.0) |
| 14 | 6.5 | 471 | 0.65M | 724 | 18.8 | 0 | 3.8 | (±0.6) |
| | | | | | | 1 | 15.9 | (±0.6) |
| | | | | | | 3 | 19.2 | (±0.3) |
| | | | | | | 9 | 32.1 | (±0.5) |
| | | | | | | 12 | 36.0 | (±0.8) |

^{d}Measured according to Method B, as described herein.

Sample 13 (Example 2, Table 4A) was stored at a concentration 4 fold greater (20 mg irinotecan/mL) than samples 1-5 (Example 1) and still retained good colloidal stability, with no observable aggregation or precipitation.

TABLE 4B
Irinotecan Liposomes prepared with DEA₈SOS Trapping Agent
at a sulfate group concentration of 0.65M, pH 7.25,)^{e}

| | mg | [irinotecan]/ | Stability | Time | Mol % | | | |
|---|---|---|---|---|---|---|---|---|
| Sample | irinotecan/mL | total mol PL | Ratio | (months) | Lyso-PC | | Size | % SN38 |
| 15 | 18.8 | 471 | 724 | 0 | 2.6 | (±0.2) | 106.8 ± 18.3 | |
| | | | | 1 | 8.8 | (±1.2) | 106.3 ± 26 | 0.05 |
| | | | | 3 | 6.9 | (±0.8) | 85.9 ± 30.8 | 0.08 |
| | | | | 9 | 9.6 | (±0.5) | 97.1 ± 19.0 | 0.05 |
| | | | | 12 | 11.0 | (±0.4) | 116.1 ± 26.6 | 0.04 |
| 16 | 18.8 | 707 | 1086 | 0 | 2.0 | (±0.6) | 101.0 ± 23.0 | |
| | | | | 1 | 0.9 | (±0.1) | 112.3 ± 23.5 | 0.01 |
| | | | | 3 | 0.93 | (±0.5) | 93.2 ± 25.0 | 0.09 |
| | | | | 9 | 2.3 | (±0.1) | 99.2 ± 19.7 | 0.03 |
| 17 | 4.7 | 707 | 1086 | 0 | 2.0 | (±0.6) | 101.0 ± 23 | |
| | | | | 1 | 0.4 | (±0.2) | 112.6 ± 23.3 | 0.07 |
| | | | | 3 | 1.1 | (±0.4) | 102.4 ± 16.2 | 0.05 |
| | | | | 9 | 1.5 | (±0.2) | 99.5 ± 15.8 | 0.06 |
| | | | | 12 | 1.5 | (±0.1) | 106.2 ± 22.5 | 0.04 |
| 18 | 18.8 | 707 | 1086 | 0 | 2.0 | (±0.6) | 101.0 ± 23 | |
| | | | | 1 | 0.7 | (±0.3) | 108.1 ± 23.7 | 0.01 |
| | | | | 3 | 0.4 | (±0.4) | 100.2 ± 18.0 | 0.04 |
| | | | | 9 | 0.1 | (±0.1) | 98.1 ± 18.3 | 0.03 |
| | | | | 12 | 1.5 | (±0.1) | 100.0 ± 26.5 | 0.01 |

^{e}Measured according to Method B, as described herein.

The freshly extruded liposome sizes encapsulated either (A) TEA₈SOS at 0.65 M sulfate (113.0±23.8 nm) or (B) DEA₈SOS at 0.65 M sulfate groups (103.2±21.1 nm) (the only exception being sample 13, which had 0.45 M sulfate groups). From (A), samples 12 and 14 and from sample (B) samples 15-18 were derived, with samples 12, 14, 15, and 16 being loaded at 471 g irinotecan free base anhydrous (equivalent to 500 g irinotecan HCl anhydrous) per mol total phospholipids and samples 16-18 being loaded at 750 g irinotecan moiety (as explained above, based on the free base anhydrous) per mol phospholipid. Following purification, pH adjustment was made using 1 N HCl or 1 N NaOH as appropriate and as described in Tables 1 and 4A to either pH 6.5 or 7.25. Sample 12 was prepared as described in Example 1.

The data showed that the liposomes retain good colloidal stability up to a year at 4° C., as judged by the absence of precipitation and the relatively narrow and reproducible particle size distributions. Secondly, it is apparent that the colloidal stability was also good for more concentrated samples when stored at high pH and at elevated drug to phospholipid ratio, indicating that at irinotecan moiety concentrations equivalent to 20 mg/mL and 40 mg/mL of irinotecan hydrochloride trihydrate, the liposomes are stable and resist formation of aggregates.

In all cases, irinotecan was stably entrapped in liposomes with low leakage and low conversion to the active cytotoxic SN-38 (i.e., relative amount of SN38 by comparison to irinotecan and SN38); less than 0.5 mol % in all cases, and with the exception of sample 12, less than 0.1 mol % SN-38. Data were obtained by “Drug Retention and Stability” method and “Drug Analysis” method described herein.

Increased levels of lyso-PC were measured in samples that had been adjusted to pH 6.5 and prepared at a ratio of 471 g irinotecan moiety (as explained above, using an equivalent amount of 500 g irinotecan HCl anhydrous) per mole of phospholipid, reaching 36-37 mol % (of the total phosphatidylcholine) for samples 12 and 14, whereas adjustment of the pH to 7.25 rendered the liposomes less susceptible to lyso-lipid formation, with lyso-PC levels approaching only 11 mol % (of the total phosphatidylcholine) after one year for Sample 15.

Changing the liposomal pH from 6.5 to 7.25 had no detrimental effect on colloidal stability or drug leakage.

### Example 3: Storage Stability of Stabilized Irinotecan Liposomes with Varying Amounts of TEA

The effect of changing the residual amount of the substituted ammonium in the drug loaded irinotecan SOS liposome was evaluated by making multiple irinotecan SOS liposomes containing varying amounts of the encapsulated residual substituted ammonium ion, storing these irinotecan SOS liposomes under refrigeration at 4° C. for 6 months and then measuring the amount of lyso-PC (in mol %) in these irinotecan SOS liposomes.

The data demonstrated that reducing the amount of substituted ammonium ion within irinotecan SOS liposomes results in lower levels of lyso-PC after 6 months of refrigerated storage at 4° C. (e.g., compared to Sample 12 in Example 1). In particular, irinotecan SOS liposomes having less than 100 ppm (e.g., 15-100 ppm TEA) substituted ammonium exhibited lower levels of lyso-PC formation after 6 months of refrigerated storage 4° C. (e.g., compared to Sample 12 in Example 1).

Six lots (Samples 24-29) of liposomal irinotecan sucrosofate were prepared according to certain embodiments of the invention, following the protocols described herein, having the Stability Ratios of 1046-1064, and lipid composition of DSPC, cholesterol, and MPEG-2000-DSPE at the molar ratio of about 3:2:0.015, respectively.

The amount of lyso-PC in Table 5 was determined by HPLC (Method A herein). “DL” in Table 5 is defined below. The rate of increase in lyso-PC measurements during refrigerated storage (at about 2-8° C.) ranged from about 0.0077-0.0585 mg lyso-PC/month, as estimated as the slope obtained from linear regression analysis.

TABLE 5
Irinotecan liposome preparations at pH 7.3

| | | | | | | | | | mol % |
|---|---|---|---|---|---|---|---|---|---|
| | | | DL | | Irinotecan/ | | Lyso PC | Lyso-PC | Lyso-PC |
| | D | Irinotecan | ratio | | SOS gram | TEA | initial | rate mg/ | at 180 |
| Sample | (mm) | mg/mL | g/mol | pH | equiv ratio | ppm | mg/mL^{f} | mL/month | days^{g} |
| 24 | 110 | 4.51 | 502 | 7.3 | 1.020 ± 0.012 | 16 | 0.060 | 0.0077 | 2.2 |
| 25 | 109 | 4.38 | 517 | 7.3 | 1.018 ± 0.031 | 14 | 0.059 | 0.0124 | 3.0 |
| 26 | 109 | 4.43 | 481 | 7.3 | 0.963 ± 0.008 | 39 | 0.148 | 0.0309 | 6.9 |
| 27 | 107 | 4.43 | 469 | 7.3 | 0.965 ± 0.019 | 79 | 0.081 | 0.0313 | 5.4 |
| 28 | 108 | 4.43 | 487 | 7.3 | 0.983 ± 0.021 | 18 | 0.060 | 0.0126 | 2.8 |
| 29 | 112 | 4.43 | 503 | 7.3 | 0.907 ± 0.009 | 100 | 0.110 | 0.0585 | 10.1 |

^{f}Measured according to Method A, as described herein.
^{g}Measured according to Method A, as described herein.

The liposomes (100-115 nm) were obtained by extrusion of the lipid dispersed in a TEA-SOS solution (0.4-0.5 M sulfate) through 100-nm polycarbonate membranes (Nuclepore), purified from extraliposomal TEA-SOS by tangential flow diafiltration buffer exchange against osmotically balanced dextrose solution, loaded with irinotecan by raising the temperature to 68° C., and stirring for 30 minutes, quickly chilled, and purified from extraliposomal TEA and any unencapsulated drug by tangential flow diafiltration buffer exchange against buffered physiological sodium chloride solution. The irinotecan sucrosofate liposome composition was filter-sterilized by passage through the 0.2-μm membrane filters, aseptically dispensed into sterile glass vials, and incubated under refrigeration conditions (5±3° C.). At the refrigerated storage times of approximately 0, 3, 6, 9, and in some cases, 12 months, duplicate vials of each lot were withdrawn and analyzed for the amount of accumulated lyso-PC using HPLC method with evaporative scattering detector. The liposome compositions were also characterized by the particle size, irinotecan and liposome phospholipid concentration, pH of the liposome composition, irinotecan/sucrosofate gram-equivalent ratio (irinotecan/SOS ratio) and residual triethylammonium (protonated TEA) (as triethylamine). The mean particle size (D) and polydispersity index (PDI) were determined by DLS method using Malvern ZetaSizer NanoZS™. Irinotecan concentration in the liposome compositions was determined by HPLC. Total phospholipid was determined spectrophotometrically by the blue phosphomolybdate method after digestion of the liposomes in sulfuric acid/hydrogen peroxide mixture.

Drug/lipid (DL in Table 5) ratio was calculated by dividing the drug amount (as free base anhydrous) in g by the molar amount of liposome phospholipid in the liposome preparation. Liposomally-entrapped SOS was quantified after passage of the liposomes through a Sephadex G-25 gel-chromatography column (PD-10, GE Healthcare) eluted with normal saline. To determine the Irinotecan/SOS gram-equivalent ratio, 0.1-mL aliquots of the eluted liposome fractions, in triplicate, were mixed with 0.05 mL of 70% perchloric acid, hydrolyzed at 95-100° C. for 1 hour, neutralized with 0.8 mL of 1 M sodium acetate, filtered to remove insoluble lipid products, and the amount of sucrosofate-derived sulfate groups in the filtrates was quantified by turbidimetry using barium-PEG reagent essentially as described under Methods. Another set of triplicate aliquots of the same liposome eluates was lysed in 70% acidified (0.1M HCl) aqueous isopropanol and assayed for irinotecan by spectrophotometry at 365 nm. The irinotecan/sucrosofate gram-equivalent ratio (Irinotecan/SOS ratio) was calculated in each eluted liposome fraction by dividing the measured molar concentration of the drug by the measured molar concentration of the sulfate groups. The pH was measured as described in subsection “pH Measurements.” TEA was quantified by headspace gas chromatographic (GC) separation utilizing gradient temperature elution on a capillary GC column followed by flame ionization detection (FID). Results are expressed as ppm (parts per million) of TEA. Levels of TEA are determined by external quantitation against a standard.

The data in FIGS. 4A, 4B, 5, 7, 8, 9A, and 9B was obtained from liposomal irinotecan samples prepared by loading 0.4-0.5 M TEA₈SOS trapping agent liposomes with about 400-600 mg (e.g., about 500 g) irinotecan moiety per mol total phospholipid (Stability Ratios ranging from about 1000-1200) and a pH after manufacturing of about 7-0.0-7.5 (e.g., about 7.25). The amount of lyso-PC in each of these liposomal irinotecan samples was measured at the time points indicated in FIGS. 4A, 4B, 5, 9A and 9B using the HPLC method of Example 8.

The lyso-PC accumulation data (in mg lyso-PC/mL liposome composition) were plotted against the storage time, as shown on FIG. 4A (Samples 24-26/Lots 1-3) or FIG. 4B (Samples 27-29/Lots 4-6). A linear correlation was observed, where the lyso-PC accumulation varied from about 0.008 mg/mL/month to about 0.06 mg/mL/month, the higher rates being characteristic for the compositions with higher TEA amounts. The amounts of lyso-PC accumulated at day 180 (about 6 months) of storage were determined from the linear approximation of the multi-point data and expressed as mol % of PC taking the molecular weight of lyso-PC equal to 523.7 g/mol. All six lots (Samples 24-29) accumulated less than 20 mol % of lyso-PC at day 180 of refrigerated storage. The lots with less than 20 ppm TEA and irinotecan/SOS gram equiv. ratio of more than 0.98 showed the least lyso-PC accumulation (less that about 0.015 mg/mL/month, lyso-PC at day 180-3.0 mol % or less); the lots with less than 80 ppm TEA accumulated lyso-PC at the rate of about 0.03 mg/mL/month, or less, and had less than 7 mol % of lyso-PC at day 180; the lot with 100 ppm residual TEA accumulated lyso-PC at the rate of about 0.06 mg/mL/month, and had about 10 mol % lyso-PC at day 180.

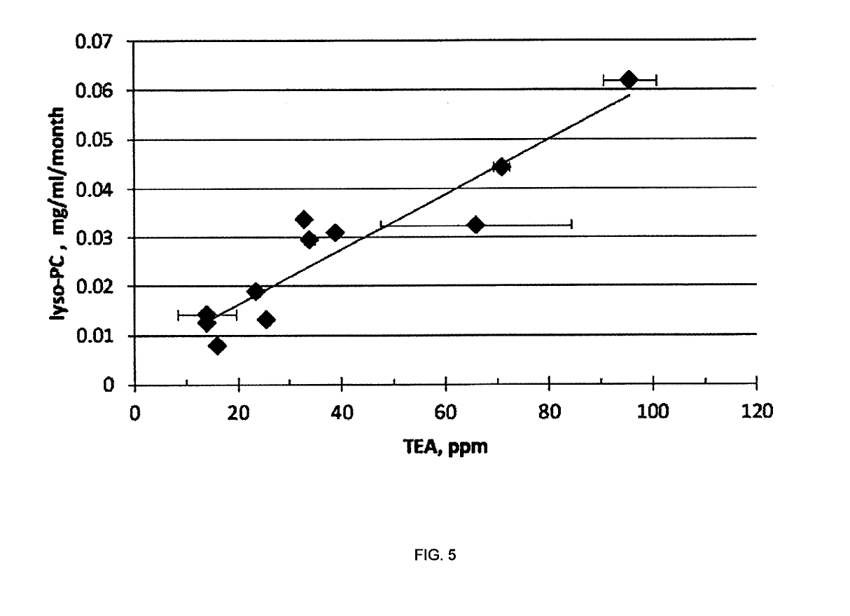

FIG. 5 is a graph showing the rates of lyso-PC accumulation (in mg/mL/month) stored at 5±3° C. plotted against TEA content (in ppm) in the stabilized irinotecan sucrosofate liposome compositions, along with the linear regression line derived from the data. Five additional lots of liposomal irinotecan sucrosofate were prepared similarly to Example 3. The preparations were stored at irinotecan moiety (as explained above, based on the free base anhydrous) of about 4.3 mg/mL irinotecan free base anhydrous per mL and periodically analyzed for lyso-PC formation and TEA content as described in Example 3. The rates of lyso-PC accumulation were calculated as the slopes of linear regression lines obtained by fitting to the lyso-PC data over storage time for each lot and plotted against the TEA content with averaged TEA readings of the BDP/DP paired lots (FIG. 4B). As follows from the graph, preparations that had about 25 ppm or less of TEA accumulated lyso-PC at the rates less than 0.02 mg/mL/month (less than 2.5 mol % lyso-PC increase over 180 day period); preparations that had less than about 70 ppm TEA accumulated lyso-PC at the rate of less than 0.033 mg/mL/month (less than 4.3 mol % lyso-PC increase over 180 day period), and all preparations had less than about 100 ppm of TEA and accumulated lyso-PC at the rate of less than 0.062 mg/mL/month (less than 8.0 mol % lyso-PC increase over 180 day period).

Samples 24, 25, and 28 each have less than 20 ppm (e.g., about 10-20 ppm) substituted ammonium ion (protonated TEA) and have the lowest amounts of lyso-PC observed after 6 months of refrigerated storage at 4° C. (2.2-3 mol % lyso-PC). Comparing samples 26 and 27, increasing the amount of residual substituted amine trapping agent counter-ion (e.g., protonated TEA) in the irinotecan SOS liposomes from about 39 ppm to 79 ppm (a 103% increase) was accompanied by an unexpected drop on the amount of lyso-PC observed after 180 days (from 6.9 mol % to 5.4 mol %, a 22% reduction in lyso-PC). However, further increasing the amount of residual substituted ammonium ion (e.g., protonated TEA) in the irinotecan SOS liposomes from 79 ppm (Sample 27) to 100 ppm (Sample 29) (i.e., a 27% increase) was accompanied by an additional 87% increase (i.e., from 5.4 mol % in Sample 27 to 10.1 mol % in Sample 29) in the amount of lyso-PC observed after 6 months of refrigerated storage at 4° C.

### Example 4: Preparation and Analysis of Irinotecan Sucrosofate

An amount of 1.64 g of irinotecan hydrochloride trihydrate was added to 160 mL of water acidified with 0.008 mL of 1 N HCl, and heated on a 65° C. water bath with stirring until the drug was dissolved. Five mL of 0.46 M (based on sulfate concentration) triethylammonium sucrosofate were added with intensive stirring, and stirred for five minutes more. A yellowish oily precipitate solidified into a brittle mass after overnight storage at 4-6° C. The mass was triturated with a glass rod to give fluffy off-white precipitate and incubated under refrigeration for 25 days. The precipitate was separated by centrifugation, and the supernatant solution was discarded. The pellet was resuspended in five volumes of deionized water, and precipitated by centrifugation; this washing step was repeated two more times until the pH of the suspension was about 5.8. Finally, the pellet was resuspended in an equal volume of deionized water to give about 26 mL or the product, having an irinotecan content of 46.0 mg/mL (free base) (yield 84% of theory). An aliquot of the product was solubilized in 1 N HCl and analyzed for irinotecan (by spectrophotometry at 365 nm in 70% aqueous isopropanol-0.1 N HCl) and for sulfate after hydrolysis in a diluted (1:4) perchloric acid using a barium sulfate turbidimetric assay. The molar ratio of irinotecan to SO₄ was found to be 1.020±0.011. Aliquots of the irinotecan sucrosofate suspension were added to deionized water to the final drug salt concentration of 0.93, 1.85, and 3.71 mg/mL. The samples were incubated with agitation at 4-6° C. for 22 hours, the solid material was removed by centrifugation for 10 min at 14000 g, and the supernatant fluid was analyzed for irinotecan by spectrophotometry. The concentration of irinotecan in solution was found to be 58.9±0.90 micro-g/mL, 63.2±0.6 micro-g/mL, and 63.4±1.3 micro-g/mL, respectively, that, on average, corresponds to an irinotecan sucrosofate molar solubility of 1.32×10⁻⁵ M.

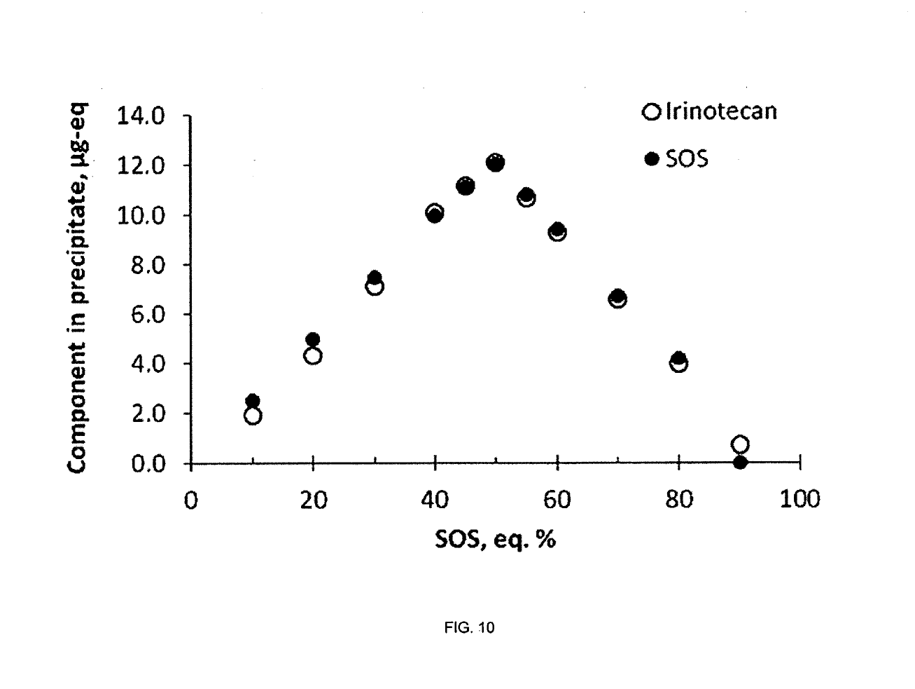

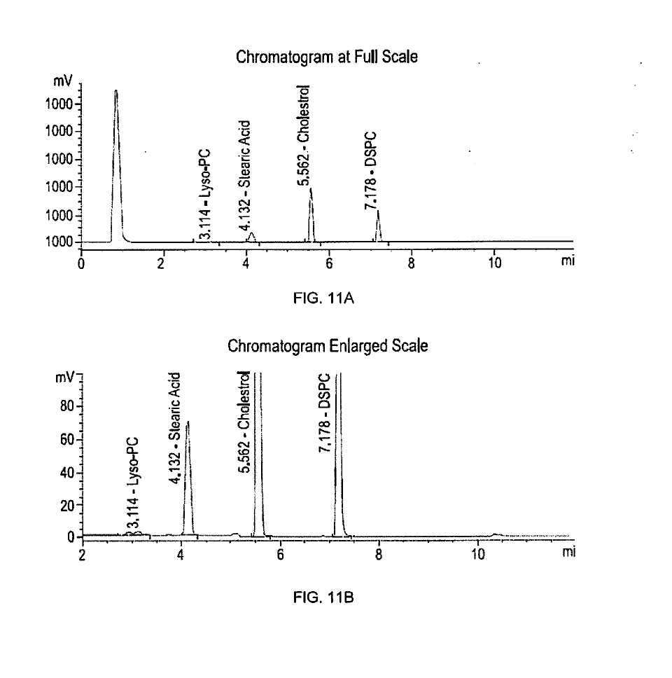

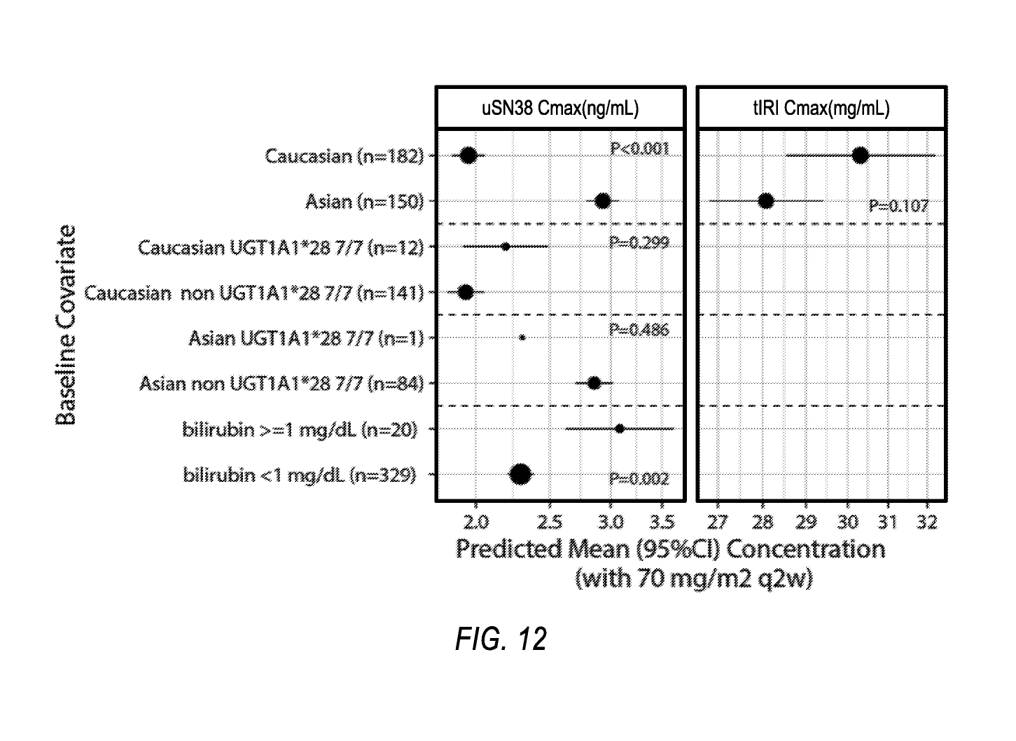

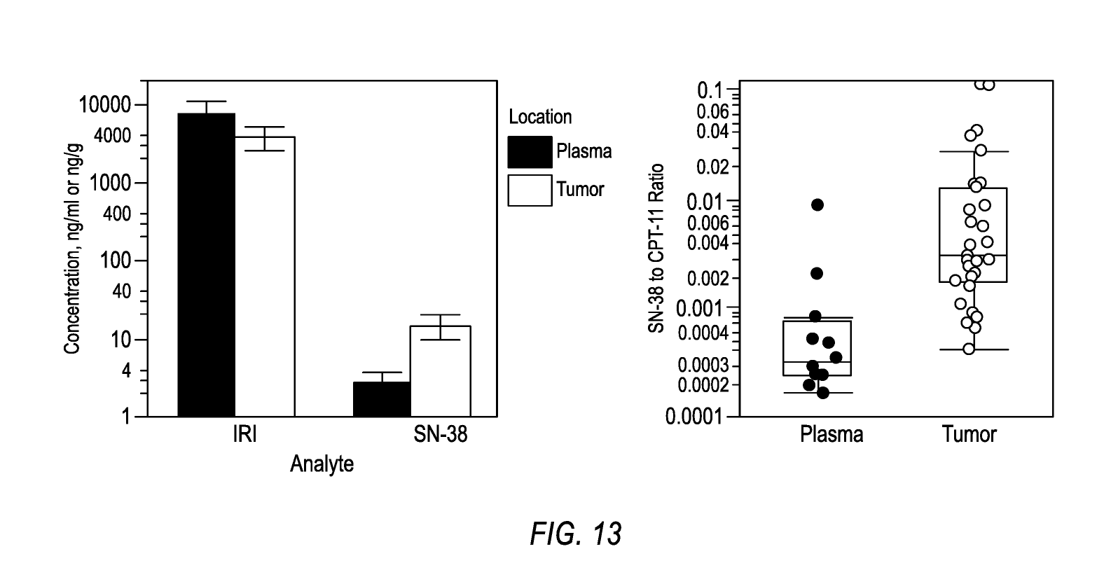

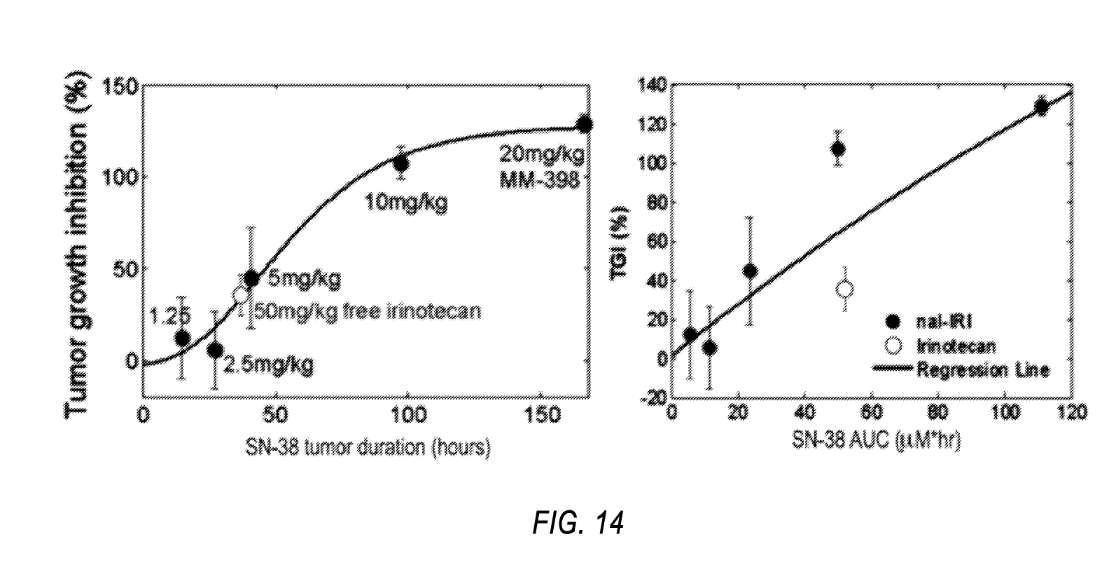

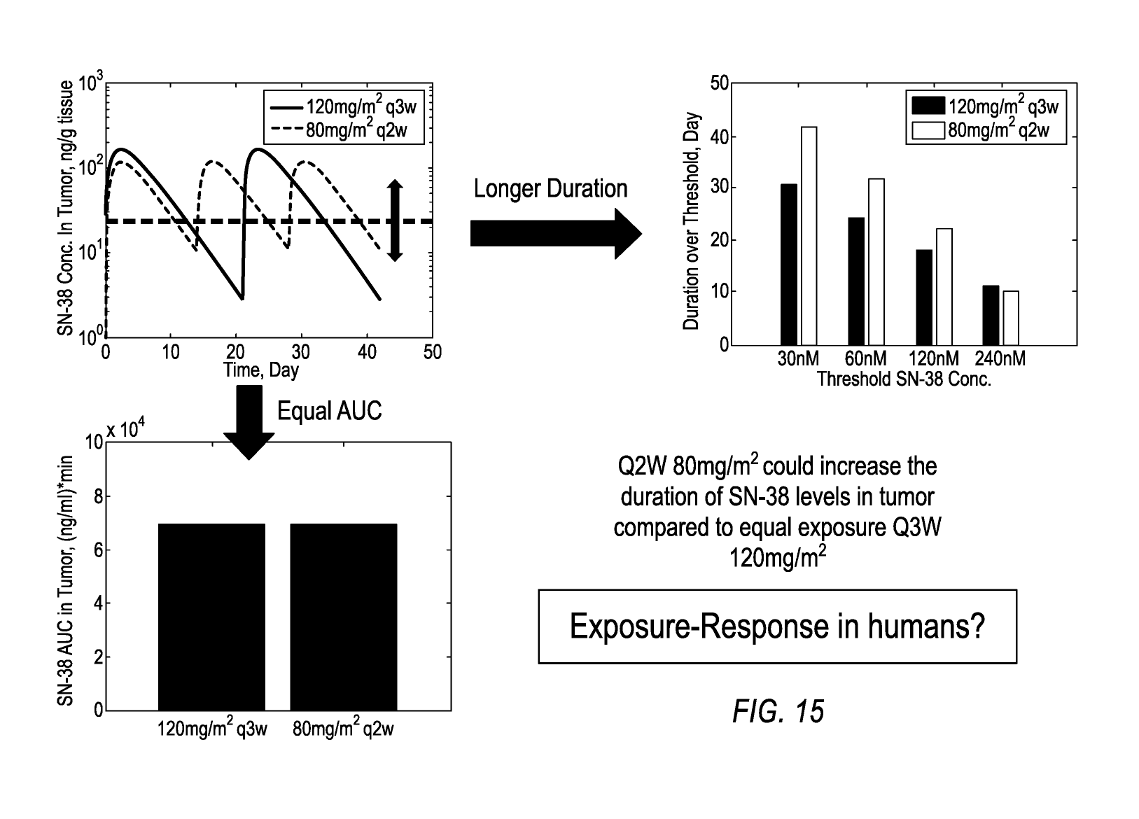

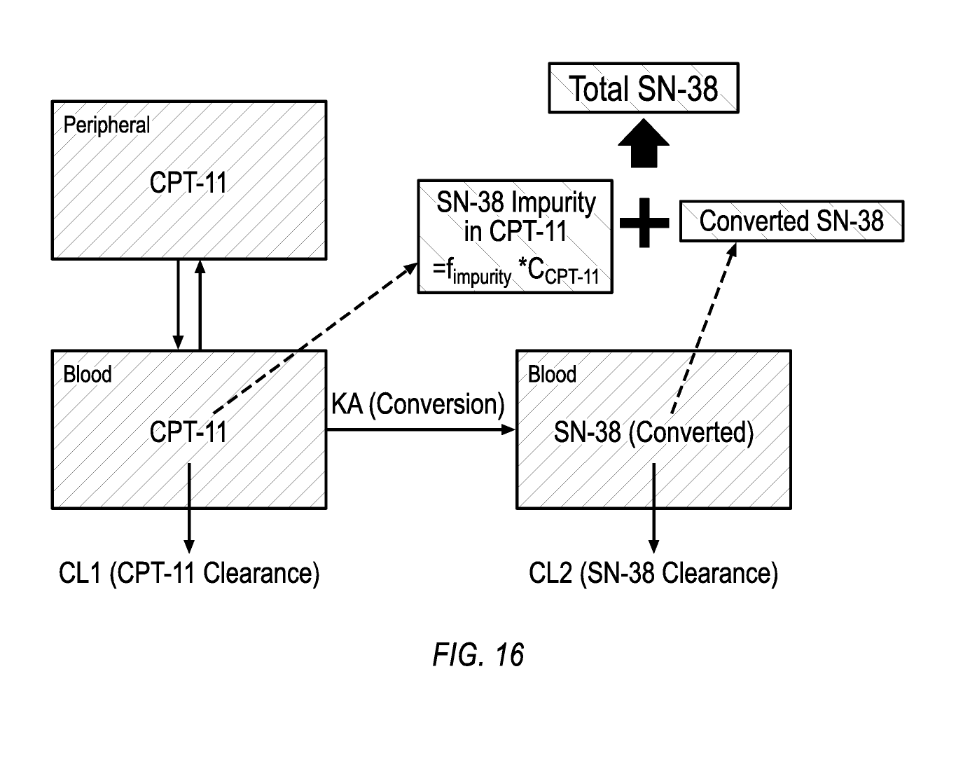

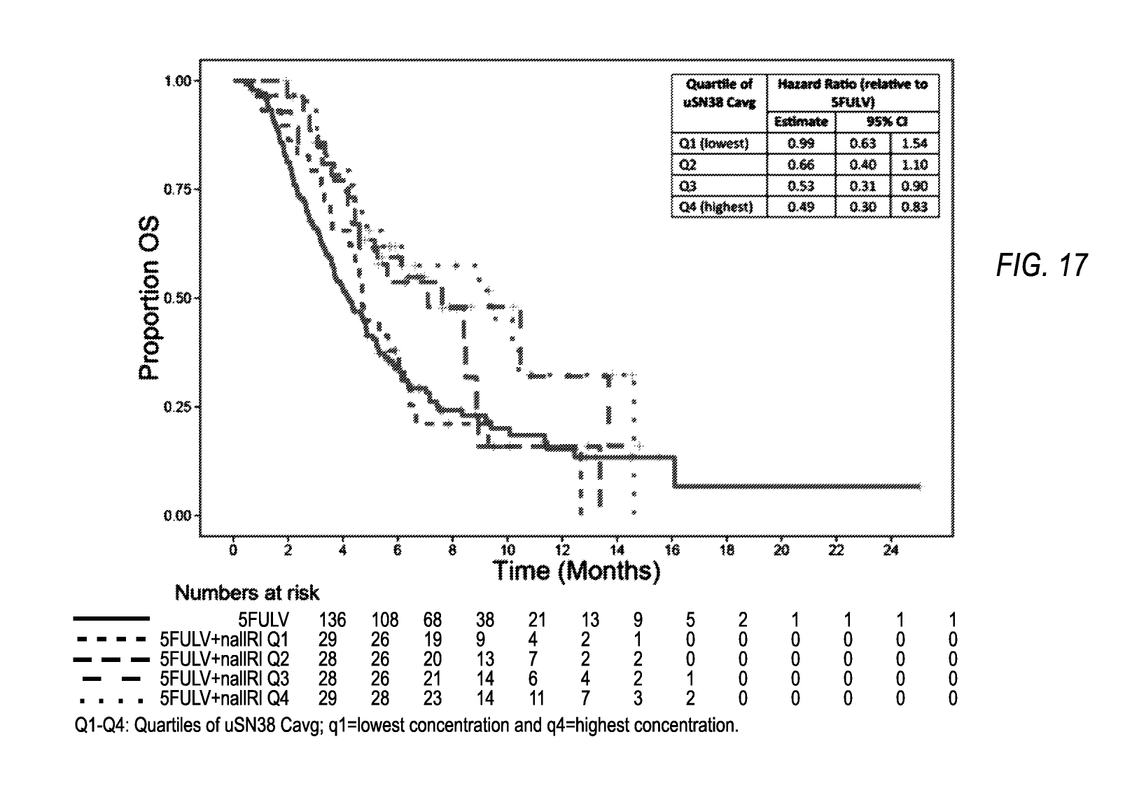

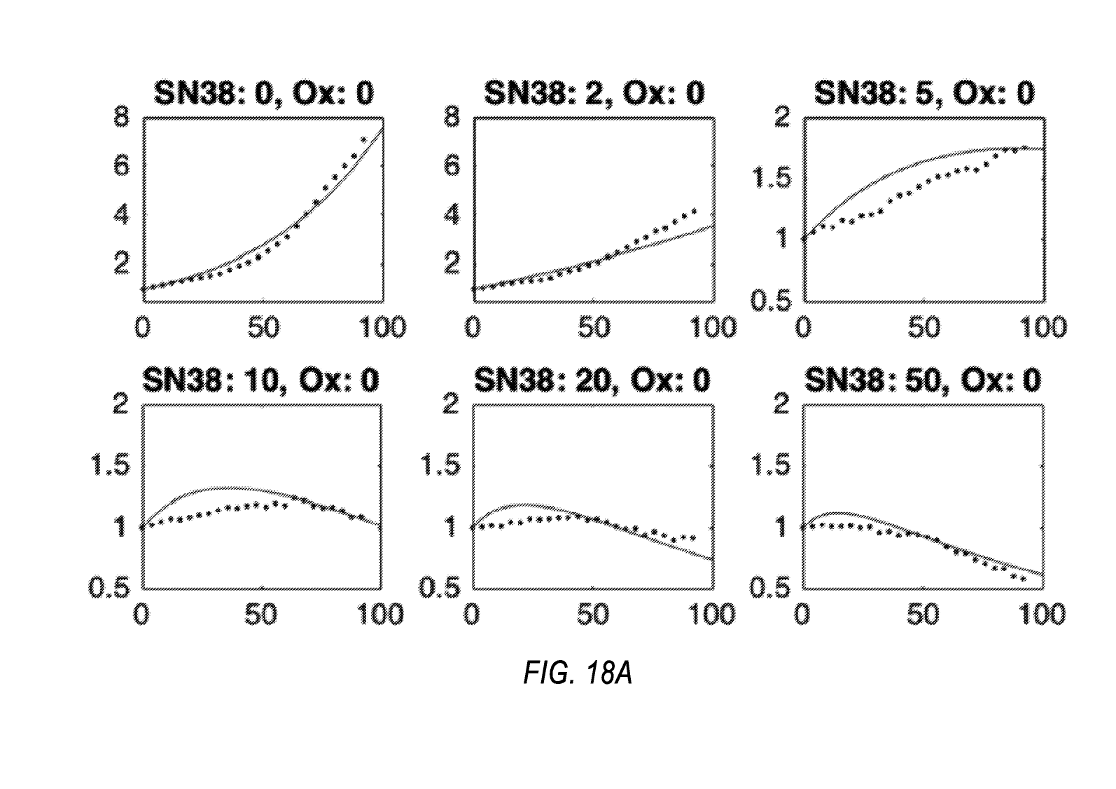

FIG. 10 is a graph showing gram-equivalent amounts of irinotecan and sucrosofate in the precipitate formed by combining irinotecan hydrochloride and triethylammonium sucrosofate in aqueous solution at various proportions of sucrosofate (SOS) as described in Example 4.

When a solution of irinotecan hydrochloride is combined with liposomes containing triethylammonium sucrosofate, a hydrogen ion can be scavenged and an irinotecan sucrosofate salt can be formed. To study the reaction between irinotecan and triethylammonium sucrosofate, we prepared 25 mM (16.93 mg/mL) aqueous solution of irinotecan hydrochloride trihydrate USP and 250 meq/L (31.25 mM) solution of triethylammonium sucrosofate (TEA-SOS) (essentially as described in the “Methods” section). Aliquots of irinotecan hydrochloride solution were diluted with water, heated to 65° C., and combined with aliquots of TEA-SOS solution to produce a series of irinotecan-SOS gram-equivalent ratios between 9:1 and 1:9, at the overall gram-equivalent concentration of both compounds together equal 25 meq/L. The samples were quickly mixed by vortexing, incubated at 65° C. for 30 minutes, chilled in ice-water, and allowed to equilibrate overnight at 4-6° C. In all samples, precipitation was observed. The next day, the samples were centrifuged at 10000×g for 5 minutes and at 14000×g for another 5 minutes, and clear supernatant fluid (over a loose, copious white to slightly tan precipitate) was isolated and analyzed for the amounts of non-precipitated irinotecan and SOS essentially as described in the Examples to determine the amount and composition of the precipitate. The results were plotted against the gram-equivalent percent of SOS in the sample (FIG. 10). In the range of 20-80 equivalent % of SOS the graphs for both components consisted of two linear branches that met at the value of 50 equivalent %, indicating that irinotecan and sucrosofate formed an insoluble salt with the stoichiometry of one irinotecan molecule per one sulfate ester group of sucrosofate (that is, eight molecules of irinotecan (IRI) per one sucrosofate (SOS) molecule):

8IRI.HCl+TEA₈SOS→(IRI.H)₈SOS↓+TEACl

Despite pronounced differences in the molecular size and shape of a protonated irinotecan molecule and a sucrosofate anion, their salt surprisingly kept close stoichiometry—eight molecules of protonated irinotecan for one sucrosofate molecule—even under the large excess of either component (FIG. 10). Thus, irinotecan sucrosofate can exist in the liposome in a poorly soluble, precipitated, or gelated form. The fact that the precipitating salt keeps its strict stoichiometry allows the process to advance to the point when mostly all or essentially all sulfate groups of sucrosofate are bound to the drug molecules. Consistent with the irinotecan-sucrosofate gram-equivalent ratio measurements of Example 6, the process of irinotecan loading to obtain a stable liposome of the present invention, in some embodiments, can comprise liposomal precipitation of the stoichiometric drug salt until at least 90%, at least 95%, or even at least 98%, and in some cases, essentially all free liposomal sucrosofate is depleted from the liposomal aqueous phase through precipitation and/or gelation of its irinotecan salt.

### Example 5: Various Irinotecan Liposomes

All the experiments for this example were conducted using a 25 mm extruder, hollow fibers, or tangential flow filtration (TFF) set-up for the initial diafiltration step, micro scale drug loading, and a TFF set-up for the final diafiltration followed by EAV filtration. Due to the limited volume of the drug loaded material, the final filtration after dilution was done using a 20 cm² EAV filter in a biosafety cabinet instead of two EBV filters.

TABLE 6

| | 31a | 31b | 32a | 32b | 33 | 34 |
|---|---|---|---|---|---|---|
| Sample | (2a) | (2b) | (3a) | (3b) | (4) | (5) |
| Encapsulated | 4.56 | 4.68 | 4.65 | 4.58 | 5.2 | 5.1 |
| Irinotecan | | | | | | |
| Concentration (mg/mL) | | | | | | |
| % Encapsulated | 98.4 | 99.2 | 98.2 | 99.3 | 99.7 | 99.8 |
| Irinotecan (%) | | | | | | |
| DSPC:cholesterol mol | 3.03:1.00 | | 2.96:1.00 | | 3:1 | 3:1 |
| ratio | | | | | | |
| Irinotecan:phospholipid | 486 | 486 | 458 | 458 | 502 | 481 |
| ratio (mg/mol) | | | | | | |
| pH | 7.28 | 7.28 | 6.41 | 6.41 | 7.3 | 7.3 |
| Particle Size | 90-130 (0.05) | | | | 110 (0.10) | 109 (0.05) |
| Measurement | | | | | | |
| (USP729) (nm) (PDI) | | | | | | |
| Lyso PC Concentration | <0.060 | 0.175 | 0.076 | 0.573 | 0.24 | 0.79 |
| (mg/mL) | | | | | | |
| Lyso-PC concentration | 4.04 | | 12.72 | | 5.11 | 16.43 |
| (mol %)^{h} | | | | | | |

^{h}Measured according to Method A, as described herein.

Referring to Table 6, a series of different irinotecan liposomes were prepared having different amounts of lyso-PC. Unless otherwise indicated, the irinotecan liposomes encapsulated irinotecan sucrose octasulfate in a vesicle consisting of DSPC, cholesterol, and MPEG2000DSPE in about a 3:2:0.015 mole ratio.

Sample 30 (lot 1) was obtained by preparing the liposomes as described in Example 1 (except as indicated in this Example) and then holding the extruded liposomes for 8 hours at 72° C. after liposome extrusion, pH adjusted to 6.2-6.9 at the end of 8 hours, resulting in a composition with about 45 mol % lyso-PC (i.e. about 1.7 mg/mL). The time of MLV preparation was considered as time 0. This experiment was performed using an aliquot from the baseline experiment 1. The composition of sample 30 (lot 1) was prepared with liposomes having a lower DSPC:cholesterol mol ratio (about 2:1 instead of 3:1 in other samples). The resulting irinotecan liposome composition had a high level of lyso-PC (i.e., greater than 1 mg/mL and greater than 40 mol % lyso-PC).

Samples 31a and 31b (lots 2a and 2b) were prepared using the process of Example 1 with modifications to test the effect of increasing the TEA-SOS solution concentration in the liposomes prior to irinotecan drug loading and the effect of decreasing the irinotecan drug loading ratio by 15% on the characteristics of the resulting irinotecan liposome compositions. The material of sample 31a (2a) was obtained by forming liposomes having vesicles comprising DSPC and cholesterol (in the ratio provided in Table 6) encapsulating a solution of TEA-SOS at a 0.5 M sulfate group concentration to form multilamellar vesicles (MLVs) and contacting these liposomes with irinotecan hydrochloride solution in the amount of 510 g irinotecan free base anhydrous/mol of PL to load the drug into the liposomes. The material of sample 31b (2b) was obtained by maintaining the liposome composition of sample 31a (2a) for 1 week at 40° C., then analyzing the sample again. The resulting irinotecan liposome compositions of samples 31a and 31 b (2a and 2b) both contained very low levels of lyso-PC (i.e. less than about 0.06 mg/mL or 4 mol % in sample 31a (2a) and about 0.175 mg/mL in sample 31b (2b)).

Samples 32a and 32b (lots 3a and 3b, respectively) were prepared using the process of Example 1, with modifications selected to study the combined effect of formulation buffer pH and decreased irinotecan drug loading ratio. The material of sample 32a (3a) was obtained by forming liposomes having vesicles comprising DSPC and cholesterol (in the ratio provided in Table 5) encapsulating a solution of TEA-SOS solution to form MLVs and contacting these liposomes with irinotecan to load the drug into the liposomes, forming irinotecan sucrose octasulfate within the liposome at the irinotecan drug loading ratio indicated in Table 6 (lower irinotecan drug loading ratio than samples 33 (4) and 34 (5)) in a buffer selected to provide a pH of about 6.50 (instead of a pH of about 7.25 in sample 30 (1)). The material of sample 32b (3b) was obtained by maintaining the composition of sample 3a for 1 week at 40° C., then analyzing the sample again. The resulting irinotecan liposome compositions 32a(3a) and 32b (3b) both contained low levels of 0.076 mg/mL and 0.573 mg/mL lyso-PC, respectively.

Samples 33 (4) and 34 (5) were prepared according to the methods described in Example 3, except as otherwise indicated. The material of sample 33 (4) and 34 (5) was obtained by forming liposomes having vesicles comprising DSPC and cholesterol (in the ratio provided in Table 6) encapsulating a solution of TEA-SOS solution to form MLVs and contacting these liposomes with irinotecan to load the drug into the liposomes, forming irinotecan sucrose octasulfate within the liposome at 500 g irinotecan moiety (based on the free base anhydrous)/mol phospholipid in a buffer selected to provide a pH of about 7.25 (instead of a pH of about 6.5 in samples 3a and 3b). The resulting irinotecan liposome compositions 3a and 3b both contained low levels of 0.24 mg/mL and 0.79 mg/mL lyso-PC, respectively.

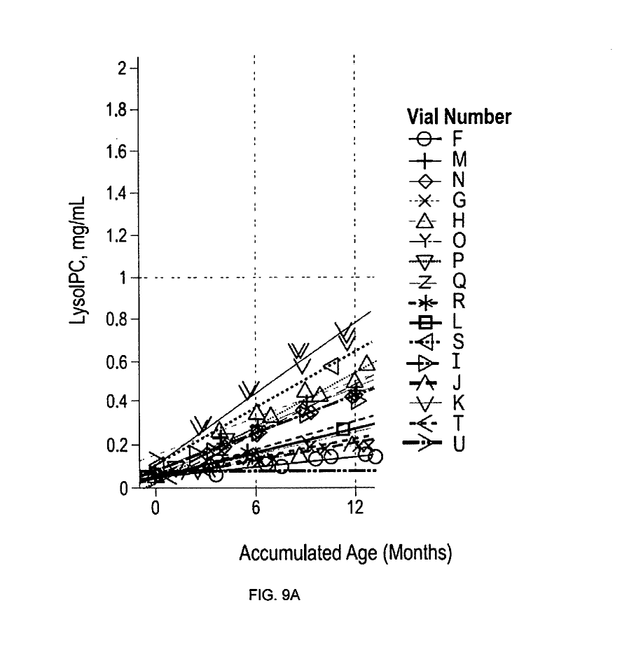

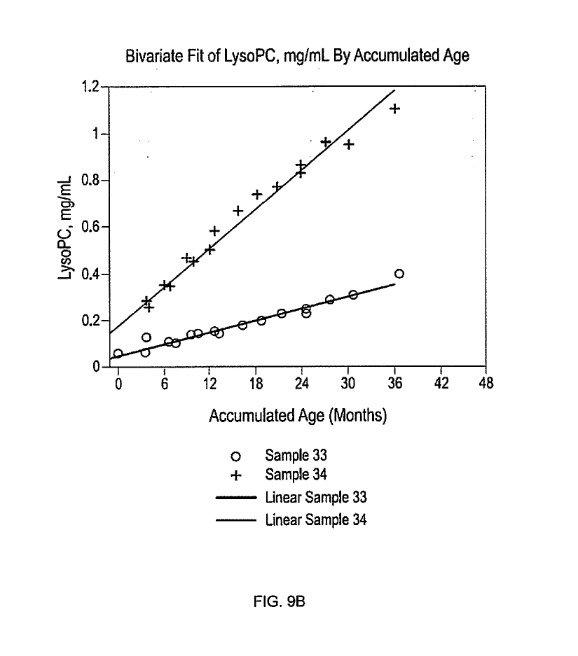

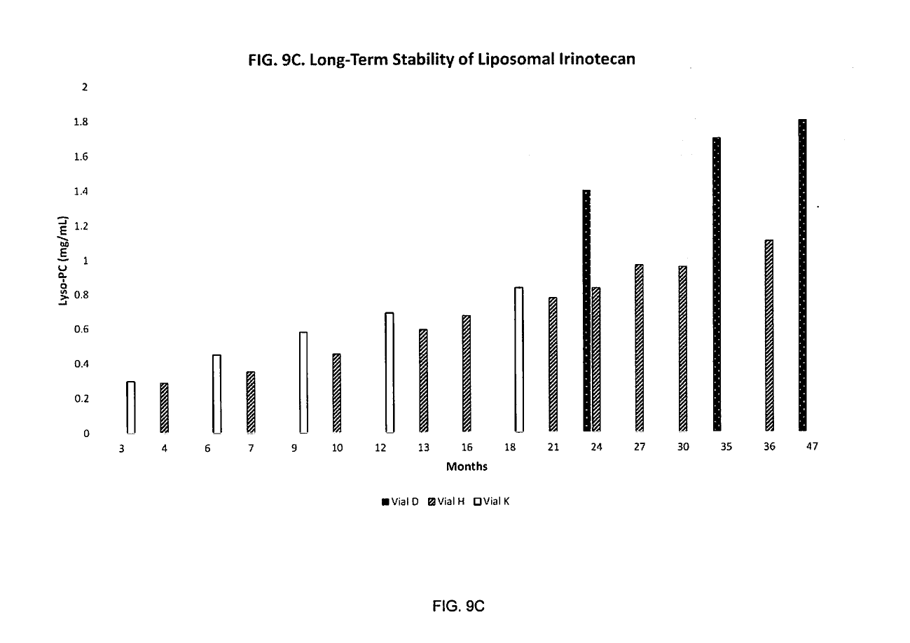

FIG. 9B is a graph showing the amount of lyso-PC measured in sample 33(4) (circles, lower line) and sample 34(5) (“+” data points, upper line). The rate of lyso-PC formation was higher in Sample 34 (5) than Sample 33 (4). The linear fit to the data points in FIG. 9B was as follows:
Sample 33 (4): lyso-PC, mg/mL=0.0514+0.0085*Accumulated Age (months)
Sample 34 (5): lyso-PC, mg/mL=0.1767+0.0280*Accumulated Age (months)
   The total lyso-PC concentration of the irinotecan liposome preparations in Samples 33 and 34 were 0.24 mg/mL and 0.79 mg/mL at 22 months, respectively.

### Example 6: Storage of Liposomal Irinotecan Preparations

Multiple liposomal irinotecan preparations were obtained according to Example 3, except as otherwise indicated, and placed on long term stability and analyzed over 12-36 months of storage at 2-8° C. (refrigerated conditions). The irinotecan liposomes were prepared by loading irinotecan into liposomes encapsulating sucrose octasulfate (SOS) and a substituted ammonium counter ion (e.g., protonated TEA). The liposomal irinotecan liposomes in this example were prepared by a process comprising the steps of: (a) contacting a solution containing irinotecan with a trapping agent liposome from TEA₈SOS at a sulfate concentration of 0.4-0.5 M under conditions effective to load 500 (±15%) grams of the irinotecan moiety per mol total phospholipid into the trapping agent liposome and simultaneously removing the TEA cation from the trapping agent liposomes during the drug loading process, to form the irinotecan SOS liposomes, and (b) adjusting the pH of the composition comprising the irinotecan SOS liposomes to obtain an liposomal irinotecan preparation having a pH of 6.8-7.6. As summarized in the table below, the Stability Ratio of the irinotecan liposomes were between about 1000 and 1200, with diameters of about 110 nm±20% (PDI no greater than 0.1). The amount of TEA remaining in the irinotecan liposomes ranged from about 15 ppm to 100 ppm.

TABLE 7
Liposomal Irinotecan Preparations

| | irinotecan | trapping agent | | | irinotecan | | | |
|---|---|---|---|---|---|---|---|---|
| | loading | concentration | | | moiety | irinotecan | | [TEA] |
| | (g/mol total | (M sulfate | Stability | final | concentration | liposome | | concentration |
| Lot | liposome PL) | groups) | Ratio | pH | (mg/mL) | size (nm) | PDI | (ppm) |
| F | 502 | 0.430 | 1167 | 7.3 | 4.5 | 110 | 0.1 | 16 |
| G | 517 | 0.430 | 1202 | 7.3 | 4.4 | 109 | 0.04 | 14 |
| L | 459 | 0.446 | 1029 | 7.2 | 4.7 | 107 | 0.05 | 24 |
| J | 487 | 0.446 | 1092 | 7.2 | 5.1 | 108 | 0.05 | 18 |
| O | 537 | 0.467 | 1151 | 7.2 | 4.5 | 109 | 0.05 | 26 |
| H | 481 | 0.430 | 1119 | 7.3 | 4.4 | 109 | 0.05 | 39 |
| I | 469 | 0.446 | 1052 | 7.3 | 4.4 | 107 | 0.04 | 79 |
| M | 514 | 0.467 | 1102 | 7.2 | 5.2 | 108 | 0.05 | 33 |
| N | 523 | 0.467 | 1121 | 7.2 | 4.6 | 109 | 0.04 | 35 |
| S | 514 | 0.446 | 1152 | 7.2 | 4.4 | 120 | 0.06 | 72 |
| K | 503 | 0.446 | 1128 | 7.2 | 4.3 | 112 | 0.04 | 100 |

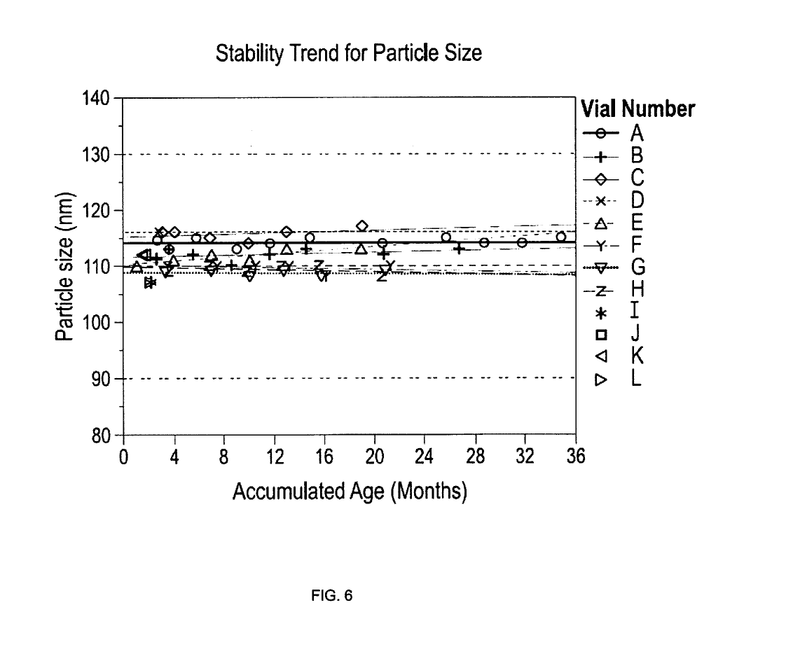

Results from analysis of the liposomal irinotecan preparations in the table above and additional lots of the liposomal irinotecan preparations obtained as described above are plotted in graphs in FIGS. 6, 7, 8, 9A, and 9B as described below.

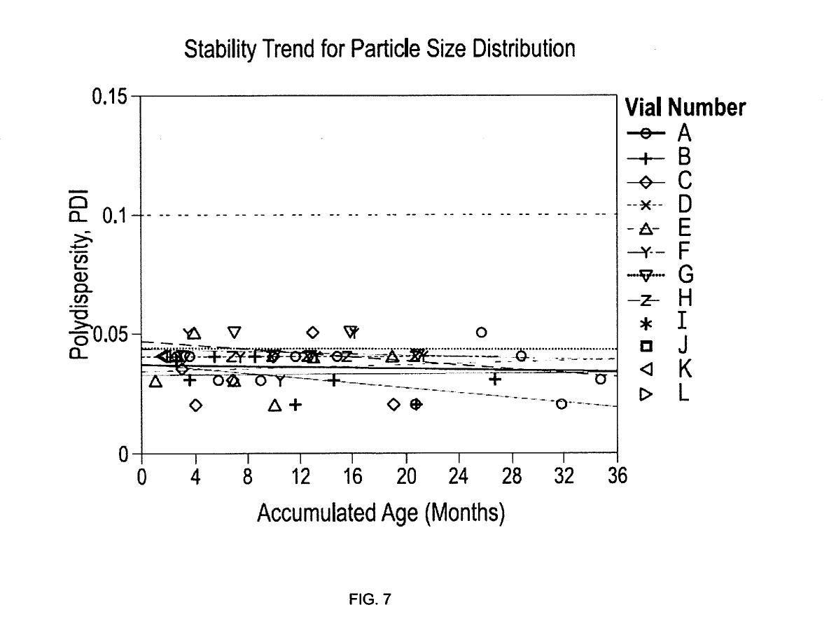

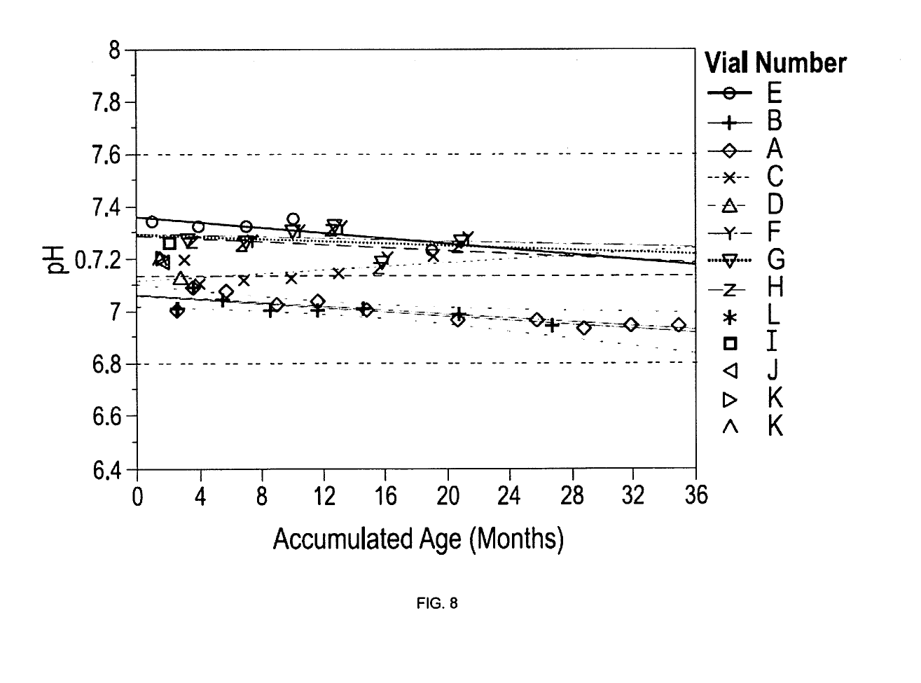

In one study, the particle size (FIG. 6) and Particle Size Distribution (FIG. 7) were measured for 12 liposomal irinotecan product preparations over 12-36 months. The PDI remained well below 0.1, and below about 0.05, for all samples. In another study, the pH (FIG. 8) was measured for 13 different liposomal irinotecan product preparations over 12-36 months. The pH remained above 6.8 during the study for all samples. In another study, the amount of lyso-PC (FIG. 9A) was measured over 12 months for 16 different liposomal irinotecan product preparations, during refrigerated storage. The amount of lyso-PC remained below 1 mg/mL for all samples.

For the purpose of determining the irinotecan free base concentration in the liposomal irinotecan product embodiment at different time points of storage, irinotecan free base is quantified as provided in the “Example” section. For the purpose of determining the lipid composition of the liposomal irinotecan product embodiment at different time points of storage, lipids are quantified using standard HPLC methodologies that are standard in the art.

For the purpose of determining the mean particle size (D) and polydispersity index (PDI) of liposomes of the liposomal irinotecan product embodiment at different time points of storage, the DLS method in conjunction with a Malvern ZetaSizer Nano ZS' was used.

For the purpose of determining the presence of lyso-PC in the liposomal irinotecan product embodiments at different time points of storage, lyso-PC is quantified as described in the “Examples” section. Additionally, it is also contemplated within the context of the present invention that lyso-PC may be quantified by HPLC as described in the specification.

### Example 7: Topotecan and Vinorelbine Liposomes

The aim of this (comparative) storage stability study was to determine any changes in the physical and chemical stability of topotecan (TPT) liposomes and vinorelbine (VNB) liposomes prepared with a sucrose octasulfate trapping agent, when stored at 4° C. Specifically, the study examined whether, during liposome manufacture, reducing the sucrose octasulfate (SOS) trapping agent concentration from 0.6 M to 0.45 M sulfate groups, while maintaining topotecan or vinorelbine to phospholipid ratio as indicated below per mol phospholipid, would have an effect on the amount of lyso-PC present in the liposome samples. Similarly, the effect of increases in the pH from 6.5 to 7.5 was examined, to determine whether this pH increase reduced the presence of lyso-PC in the liposome compositions. TPT and VNB were encapsulated with a SOS trapping agent in liposomes containing DSPC, cholesterol (Chol), and PEG-DSPE in a 3:2:0.015 molar ratio. The formulation parameters investigated include: solution pH (6.5-7.5), concentration of the sucrose octasulfate trapping agent during liposome preparation (0.45-0.6 M sulfate), the drug encapsulated (TPT or VNB), and the drug to lipid ratio (500 g TPT HCl per mol phospholipid during liposome loading; for VNB, from 350 to 450 g VNB moiety per mol phospholipid during liposome loading). The various physicochemical properties of the liposomes that were monitored during this stability study were: liposome size, drug to phospholipid ratio, drug encapsulation efficiency, general appearance, and lyso-lipid formation.

DSPC, cholesterol (Chol), and PEG-DSPE were weighed out in amounts that corresponded to about a 3:2:0.015 molar ratio, respectively (790.15 mg/257.8 mg/14.0 mg). The lipids were dissolved in chloroform/methanol (4/1, v/v), mixed thoroughly, and divided into 2 aliquots (A and B). Each sample was evaporated to dryness using a rotary evaporator at 60° C. Residual chloroform was removed from the lipids by placing under vacuum (180 μtorr) at room temperature for 12 hours. The dried lipids were dissolved in ethanol at 60° C., and pre-warmed TEA₈SOS of appropriate concentration was added so that the final alcohol content was 10% (v/v). The total phospholipid concentration was approximately 75 mM. The lipid solution was extruded through 0.1 μm polycarbonate membranes (Nuclepore™) 10 times, to produce liposomes with a typical average diameter of 95-115 nm. The pH of the extruded liposomes was adjusted as needed (with 1 N NaOH) to pH 6.5 if necessary. The liposomes were purified by a combination of ion-exchange chromatography and size-exclusion chromatography. First, Dowex™ IRA 910 resin was treated with 1 N NaOH, followed by 3 washes with deionized water, and then followed by 3 washes of 3 N HCl, and then multiple washes with water. The conductivity of the eluted fractions was measured by using a flow-cell conductivity meter (Pharmacia, Uppsala, Sweden). The fractions were deemed acceptable for further purification if the conductivity was less than 15 μS/cm. The liposome eluate was then applied to a Sephadex G-75 (Pharmacia) column equilibrated with deionized water, and the collected liposome fraction was measured for conductivity (typically less than 1 μS/cm). 40% dextrose solution was added to achieve a final concentration of 5% (w/w), and the buffer (Hepes) was added from a stock solution (0.5 M, pH 6.5) to a final concentration of 10 mM.

A stock solution of topotecan hydrochloride was prepared by dissolving 50 mg in 10 mL deionized water. Drugs were added to liposome solutions at the drug/lipid ratio indicated for each formulation in the results Table 8. For TPT loading, the pH was adjusted to pH 6.0 prior to loading. Vinorelbine was added directly from the commercial USP injection solution from the pharmacy, and the pH of the resulting mixture adjusted to 6.5 with 1 N NaOH prior to heating. Drug loading was initiated by heating the liposome/drug mixtures to 60° C. for 30 minutes. The solutions were rapidly cooled upon removal from the water bath by immersing in ice cold water. Extra liposomal drug was removed by size exclusion chromatography, using Sephadex G75 columns equilibrated and eluted with Hepes (10 mL) buffered saline (HBS), pH 6.5. The samples were analyzed for irinotecan by HPLC and phosphate by the method of Bartlett (see Phosphate Determination).

For storage, the samples were divided into 4 mL aliquots, and the pH was adjusted if necessary using 1 N HCl or 1 N NaOH, sterile filtered under aseptic conditions, and filled into sterile clear glass vials that were sealed under argon with a Teflon® lined threaded cap and placed in a thermostatically controlled refrigerator at 4° C. At defined time points, an aliquot was removed from each sample and tested for appearance, size, drug/lipid ratio, and drug and lipid chemical stability. The liposome size was determined in the diluted samples by dynamic light scattering using Coulter Nano-Sizer at 90 degree angle and presented as Mean±Standard deviation (nm) obtained by the method of cumulants.

The results from comparative stability studies are provided in Table 8.

TABLE 8
Topotecan and Vinorelbine Liposomes prepared with TEA₈SOS Trapping Agent
(0.6N SOS sulfate groups, stored at 2 mg/mL drug concentration)

| | | [gram of | | | | |
|---|---|---|---|---|---|---|
| | | drug]/total | | Time | Mol % | |
| Sample | Drug | mol PL | pH | (months) | Lyso-PCⁱ | Size ± SD |
| 19 | TPT | 500ⁱ | 6.5 | 0 | 0 | 115.0 ± 9.5 |

| | | | |
|---|---|---|---|
| 1 | 12.2 | (±0.71) | 107.3 ± 16.9 |
| 3 | 25.0 | (±0.9) | 108.4 ± 9.1 |
| 6 | 25.9 | (±0.5) | 102.3 ± 25.2 |
| 9 | 29.0 | (±1.4) | 108.6 ± 19.2 |

| | | | | | | |
|---|---|---|---|---|---|---|
| 20 | TPT | 500^{j} | 7.25 | 0 | 0 | 115.0 ± 9.5 |

| | | | |
|---|---|---|---|
| 1 | 10.0 | (±0.4) | 109.0 ± 16.8 |
| 3 | 19.0 | (±0.5) | 108.6 ± 15.8 |
| 6 | 23.3 | (±2.2) | 105.5 ± 13.6 |
| 9 | 29.4 | (±3.1) | 110.6 ± 12.1 |

| | | | | | | |
|---|---|---|---|---|---|---|
| 21 | VNB | 350 | 6.5 | 0 | 0 | 115.0 ± 9.5 |

| | | | |
|---|---|---|---|
| 1 | 2.2 | (±1.1) | 105.3 ± 16.7 |
| 3 | | | 105.8 ± 18.1 |
| 6 | 9.5 | (±1.2) | 102.8 ± 8.9 |
| 9 | 9.5 | (±0.6) | 103.4 ± 23.3 |

| | | | | | | |
|---|---|---|---|---|---|---|
| 22 | VNB | 350 | 7.25 | 0 | 0 | 115.0 ± 9.5 |

| | | | |
|---|---|---|---|
| 1 | 1.3 | (±0.1) | 105.3 ± 16.7 |
| 3 | | | 105.8 ± 18.1 |
| 6 | 5.0 | (±0.5) | 102.8 ± 8.9 |
| 9 | 5.5 | (±2.6) | 103.4 ± 23.2 |

| | | | | | | |
|---|---|---|---|---|---|---|
| 23 | VNB | 450 | 6.5 | 0 | 0 | 115.0 ± 9.5 |

| | | | |
|---|---|---|---|
| 1 | 0.3 | (±0.1) | 90.6 ± 29.6 |
| 3 | | | 104.7 ± 21.2 |
| 6 | 3.1 | (±1.1) | 106.4 ± 16.7 |
| 9 | 3.4 | (±0.3) | 133.3 ± 16.6 |

ⁱMeasured according to Method B, as described herein.
^{j}500 g topotecan HCl per mol total phospholipids

The effect of storage media pH on the production of lyso-lipid in topotecan loaded liposomes was not observed in Samples 19 and 20. Both formulations in samples 19 and 20 exhibited close to 30 mol % lyso-lipid after 9 months, even though sample 19 was stored at pH 6.5 and sample 20 was stored at pH 7.25.

In contrast to both the liposomal irinotecan, liposomal vinorelbine was more resistant to lipid hydrolysis, in that the highest amount of lyso-lipid measured was in sample 21, having 9.5 mol % lyso-lipid after 9 months. Although less pronounced, we can also detect a dependence on the Stability Ratio and storage media pH. Higher Stability Ratio resulted in reduced lipid hydrolysis (compare samples 21 to 23). A pH of 7.25 also reduced the amount of observed lipid hydrolysis (compare samples 21 to 22).

### Example 8: HPLC Method for Measuring Lyso-PC (“Method A”)

The amount of lyso-PC in the irinotecan sucrose octasulfate liposome preparations tested to obtain data in FIGS. 9A and 9B was obtained using HPLC with detection by evaporative light scattering. A suitable HPLC method (referred herein to “Method A”) is a quantitative method used to measure the amount of stearic acid, lyso-PC, cholesterol, and DSPC (1,2-distearoyl-sn-glycero-3-phosphocholine) in the drug product. The liposomes are dissociated into their individual lipid components using a methanol-tetrahydrofuran solution. The lipid components are quantitated using reverse phase high pressure liquid chromatography equipped with an evaporative light scattering detector.

### Sample and Standard Preparation

### Standard Preparation:

### Lyso-PC

A five point standard curve is prepared by diluting appropriate quantities of lyso-PC with 85:15 methanol-tetrahydrofuran to target final concentrations of 4, 8, 20, 32, and 40 μg/mL.

### Stearic Acid

A five point standard curve is prepared by diluting appropriate quantities of stearic acid with 85:15 methanol-tetrahydrofuran to target final concentrations of 2, 4, 10, 16, and 20.4 μg/mL.

### Cholesterol

A five point standard curve is prepared by diluting appropriate quantities of cholesterol with 85:15 methanol-tetrahydrofuran to target final concentrations of 90, 144, 183.7, 224.9 and 266.6 μg/mL.

### DSPC

A five point standard curve is prepared by diluting appropriate quantities of DSPC with 85:15 methanol-tetrahydrofuran to target final concentrations of 220, 352, 449, 549.8, and 651.7 μg/mL.

### Assay Control

An assay control is prepared by diluting stearic acid in diluent (85:15 methanol-tetrahydrofuran) to a target final concentration of 9.0 μg/mL and 18.0 μg/mL.

### Sample Preparation:

Samples are prepared by diluting each sample in 85:15 methanol-tetrahydrofuran solution to a target final DSPC concentration of 475 μg/mL.

### Solution Stability

The test samples standards, and assay controls have demonstrated acceptable stability in solution for up to 48 hours when stored at ambient temperature.

### Instrument and Instrument Parameters

A suitable high pressure chromatographic system equipped with an evaporative light scattering detector capable of changing gain and filter settings throughout a run, if need be, to ensure proper peak detection. The instrument operating parameters are listed in Table 9.

TABLE 9
Chromatographic Conditions

| Chromatographic | | | |
|---|---|---|---|
| Parameter | Chromatographic Conditions and Set Points | | |
| Column | Phenomenex Luna C8(2) 100 μm, 150 mm × 3 | | |
| | mm with guard column Phenomenex C8 | | |
| | 4 × 2.0 mm | | |
| Injection Volume | 20 μL | | |
| Column Temperature | 30° C. | | |
| Flow Rate | 1.0 mL/minutes | | |
| Mobile Phase A | 100 mM Ammonium Acetate pH 4.0 | | |
| Mobile Phase B | Methanol | | |
| ELSD Settings | Gas Pressure: 3.5 bar | | |
| | Temperature: 40° C. | | |
| | Time | Mobile Phase A | Mobile Phase A |
| | (minutes) | (%) | (%) |
| Gradient | 0 | 15 | 85 |
| | 3 | 8 | 92 |
| | 6 | 0 | 100 |
| | 9 | 0 | 100 |
| | 9.1 | 15 | 85 |
| | 12 | 15 | 85 |

TABLE 10
System Suitability

| Parameter | Acceptance Criteria |
|---|---|
| Elution Profile | Chromatographic profile of diluent blank, working |
| | standard, and assay control are comparable to the |
| | examples shown in the test method. |
| Plates | Average plates ≥2000 for DSPC and Cholesterol in |
| | calibration standard level 5 (n = 5 injections) |
| Tailing | Average tailing ≤1.5 for DSPC and Cholesterol in |
| | calibration standard level 5 (n = 5 injections) |
| Signal-to-noise | Signal-to-noise ≥10 for lyso-PC peak in calibration |
| | standard level 1 |
| Precision | % RSD ≤6.0 for lyso-PC, stearic acid, DSPC and |
| | cholesterol in in calibration standard level 5 (n = 5 |
| | injections) |
| Linearity | R² ≥0.99 for lyso-PC, stearic acid, DSPC and |
| | cholesterol standard calibration curves. |
| Accuracy | % Recovery = 90-110% for DSPC and cholesterol |
| | within standard calibration curves |
| Accuracy | % Recovery = 80-120% for stearic acid control |

Each lipid concentration is determined by analyzing the sample peak area to the standard curve. A second order polynomial equation (quadratic curve) trend line is used to calculate the lipid concentrations of lyso-PC and Stearic Acid. A linear trend line is used to calculate the lipid concentrations of DSPC and cholesterol.

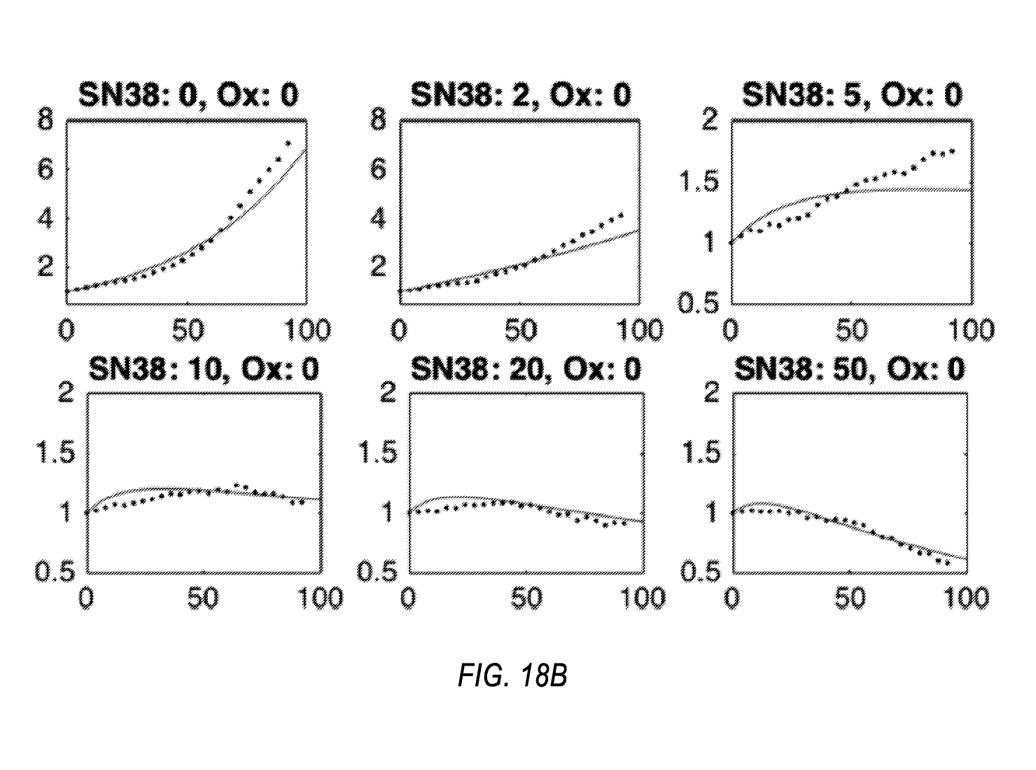

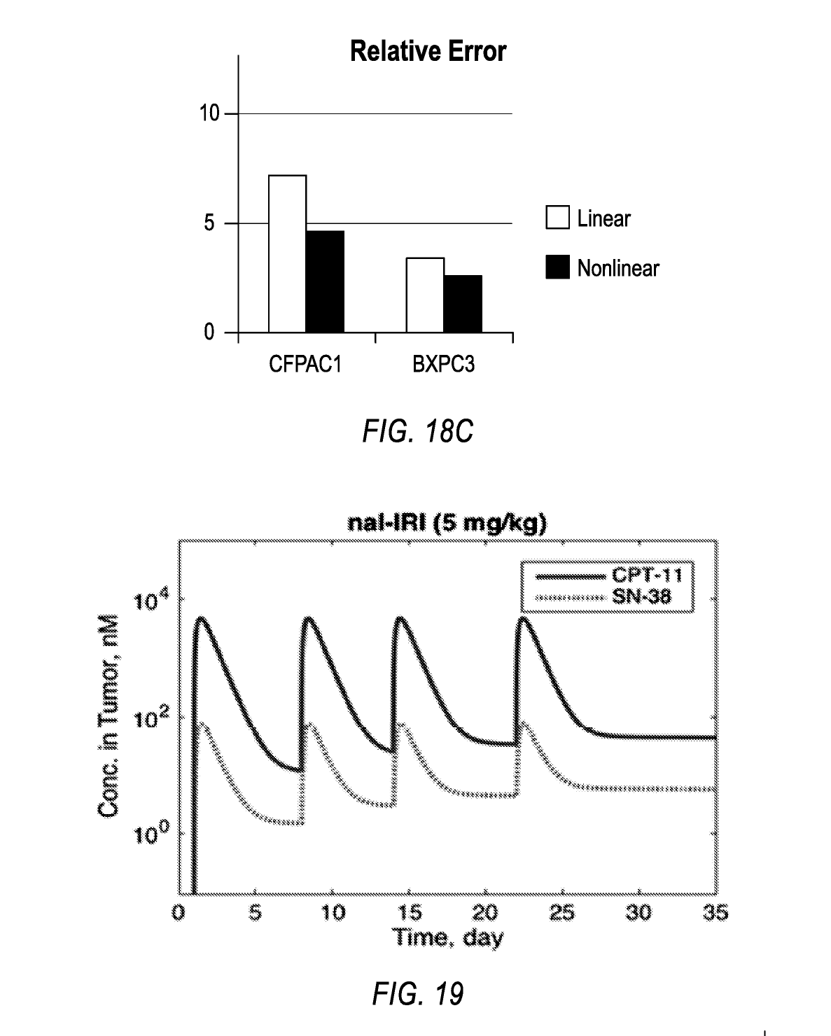

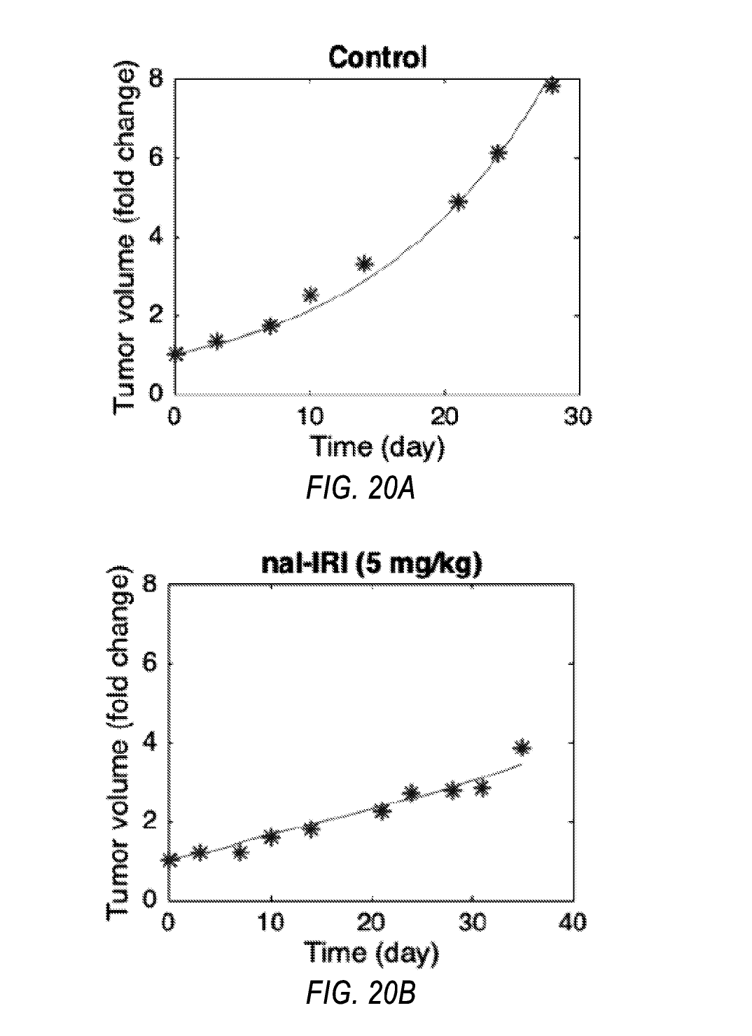

A representative chromatogram is presented in FIG. 11A and FIG. 11B.

All references cited herein are incorporated herein by reference in their entirety.

1. A storage stabilized liposomal irinotecan composition comprising irinotecan sucrose octasulfate (SOS) encapsulated in irinotecan liposomes comprising one or more phospholipids with a ratio corresponding to a total of 500 grams irinotecan moiety (±10% by weight) per mol total phospholipids, the liposomal irinotecan composition stabilized to have less than 20 mol % (with respect to total phospholipids) lyso-PC during the first 6 months of storage of the liposomal irinotecan composition at about 4° C., the liposomal irinotecan composition obtained by a process comprising the steps of:
(a) forming liposomes from triethylamine sucrose octasulfate and/or diethylamine sucrose octasulfate having a total sulfate concentration from 0.4 to 0.5 M, cholesterol and phospholipids comprising DSPC and a compound comprising polyethylene glycol and distearoylphosphatidyl ethanolamine;
(b) contacting the liposomes with a solution comprising the irinotecan moiety and made using irinotecan free base or irinotecan salt at a temperature above the transition temperature of the phospholipids in the liposomes, thereby forming a preparation of irinotecan liposomes encapsulating the irinotecan moiety in the irinotecan sucrose octasulfate in the liposomes to form the irinotecan liposomes; and
(c) adjusting the pH of the preparation of irinotecan liposomes to be from 7.0 to 7.5.

2. The liposomal irinotecan composition of claim 1, made by a process comprising steps of:
(a) forming a lipid dispersion in a solution made from TEA₈SOS having a sulfate concentration from 0.4 to 0.5 M and a pH from 5 to 7, the lipids in said dispersion being DSPC, cholesterol and MPEG-2000-DSPE in an about 3:2:0.015, respectively, mole ratio;
(b) extruding the lipid dispersion from 60 to 70° C. through at least one 0.1 μm membrane to form liposomes;
(c) substantially removing ions derived from TEA₈SOS that are outside the liposomes;
(d) contacting the liposomes at a temperature from 60 to 70° C. with a solution made using irinotecan free base or irinotecan salt, thereby forming a preparation of liposomes encapsulating irinotecan;
(e) substantially removing substances derived from the TEA₈SOS and irinotecan ingredients that are outside the liposomes; and
(f) adjusting the pH of the composition to be from 7.0 to 7.5.

3. The liposomal irinotecan composition of claim 1, where the liposomes have a mean size of 110 nm±20% as determined by dynamic light scattering and where the size is determined by the method of cumulants.

4. The liposomal irinotecan composition of claim 1, having a total irinotecan moiety content equivalent to 4.3 mg/ml irinotecan free base anhydrous.

5. The liposomal irinotecan composition of claim 2, wherein:
in step (a) the liposomes are formed from TEA₈SOS having a sulfate concentration from 0.43 to 0.47 M; and
in step (d) the solution made using irinotecan free base or an irinotecan salt has an irinotecan moiety content equivalent to 500 g (±10%) of irinotecan free base anhydrous per mole of DSPC; and
in step (f) adjusting the pH of the composition to be from 7.2 to 7.3.

6. The liposomal irinotecan composition of claim 1, containing less than 1 mol % lyso-phosphatidylcholine (lyso-PC) prior to storage at about 4° C., and 20 mol % or less (with respect to total phospholipid) of lyso-PC after 180 days of storage at about 4° C.

7. The liposomal irinotecan composition of claim 1, containing 20 mol % or less (with respect to total phospholipid) of lyso-phosphatidylcholine (lyso-PC) after 6, 9, or 12 months of storage at about 4° C.

8. The liposomal irinotecan composition of claim 1, wherein the liposomal irinotecan comprises the irinotecan liposomes in an isotonic HEPES aqueous buffer at a concentration of from 2 to 20 mM.

9. The liposomal irinotecan composition of claim 1, further comprising sodium chloride at a concentration from 130 to 160 mM.

10. The liposomal irinotecan composition of claim 1, wherein the irinotecan encapsulated in the liposomes is in a gelated or precipitated state as a sucrose octasulfate salt.

11. The liposomal irinotecan composition of claim 1, wherein the irinotecan liposomes have a diameter from 95 to 115 nm, as measured by quasi-elastic light scattering.

12. The liposomal irinotecan composition of any claim 1, comprising a total of 6.81 mg DSPC/ml, 2.22 mg cholesterol/ml, and 0.12 mg MPEG-2000-DSPE/ml, 4.05 mg/mL HEPES aqueous buffer and 8.42 mg sodium chloride/mL.

13. The liposomal irinotecan composition of any claim 1, having a pH of about 7.25, wherein the irinotecan liposomes have a diameter of 110 nm as measured by quasi-elastic light scattering.

14. The liposomal irinotecan composition of any claim 1, forming less than 1 mg/mL lyso-phosphatidylcholine (lyso-PC) after 6 months of storage at about 4° C.

15. The liposomal irinotecan composition of any claim 1, made by a process comprising steps of:
(a) forming a lipid dispersion in a solution of TEA₈SOS having a sulfate concentration of about 0.45 M and a pH of about 6.5, the lipids in said dispersion consisting of 1,2-distearoyl-sn-glycero-3-phosphocholine (DSPC), cholesterol and methoxy-terminated polyethylene glycol (MW 2000)-distearoylphosphatidyl ethanolamine (MPEG-2000-DSPE) in a mole ratio of 3:2:0.015, respectively;
(b) extruding the lipid dispersion from 60 to 70° C. through at least one 0.1 μm membrane to form liposomes;
(c) removing ions derived from TEA₈SOS that are outside the liposomes;
(d) contacting the liposomes at a temperature from 60 to 70° C. with a solution made using irinotecan hydrochloride trihydrate, to form a preparation of liposomes encapsulating about 500 g (±10%) irinotecan per mol total phospholipid;
(e) removing substances derived from the TEA₈SOS and irinotecan ingredients that are outside the liposomes; and
(f) adjusting the pH of the composition to be about 7.3.

16. The liposomal irinotecan composition of claim 1, comprising a total of less than 100 ppm of TEA or DEA.

17. The liposomal irinotecan composition of claim 1, comprising a total from 30 to 100 ppm of TEA or DEA.

18. The liposomal irinotecan composition of claim 1, wherein at least 98% of the irinotecan is encapsulated in the irinotecan liposomes after 6 months of storage at about 4° C.

19. The liposomal irinotecan composition of claim 3, having a total irinotecan moiety content equivalent to 4.3 mg/ml irinotecan free base anhydrous, a pH from 7.25 to 7.50, and the irinotecan liposomes have a total of less than 100 ppm of TEA.

20. A storage stabilized liposomal irinotecan composition comprising irinotecan sucrose octasulfate (SOS) encapsulated in irinotecan liposomes comprising one or more phospholipids with a ratio corresponding to a total of 500 grams irinotecan moiety (±10% by weight) per mol total phospholipids, the irinotecan liposomes having a mean size of 110 nm±20% as determined by dynamic light scattering where the size is determined by the method of cumulants; the liposomal irinotecan composition having a pH from 7.0 to 7.5 after manufacture and before storage, and a total irinotecan moiety content equivalent to 4.3 mg/ml irinotecan free base anhydrous and being stabilized to have less than 20 mol % (with respect to total phospholipids) lyso-PC during the first 6 months of storage of the liposomal irinotecan composition at about 4° C., the liposomal irinotecan composition obtained by a process comprising the steps of:
(a) forming liposomes from a TEA₈SOS and/or DEA₈SOS solution(s) having a total sulfate concentration from 0.4 to 0.5 M, cholesterol, DSPC and MPEG-2000-DSPE in an about 3:2:0.015, respectively, mole ratio; and
(b) contacting the liposomes with a solution comprising the irinotecan moiety and made using irinotecan free base or irinotecan salt at a temperature above the transition temperature of the phospholipids in the liposomes, thereby forming a preparation of irinotecan liposomes encapsulating the irinotecan moiety in the irinotecan sucrose octasulfate within the liposomes to form the irinotecan liposomes.
